(19) United States
(12) Patent Application Publication
(10) Pub. No.: US 2023/0066517 A1
(43) Pub. Date: Mar. 2, 2023

(54) Phospholipid Ether Conjugates as Cancer-Targeting Drug Vehicles

(71) Applicant: Cellectar Biosciences, Inc., Florham Park, NJ (US)

(72) Inventors: Jarrod Longcor, East Haven, CT (US); Anatoly Pinchuk, Fitchburg, WI (US); Randall Hoover, Florham Park, NJ (US); Zhongping Huang, Getzville, NY (US)

(21) Appl. No.: 17/642,561

(22) PCT Filed: Sep. 11, 2020
(86) PCT No.: PCT/US2020/050459

§ 371 Date: Mar. 11, 2022

Related U.S. Application Data
(60) Provisional application No. 62/899,615, filed on Sep. 12, 2019.
(60) Provisional application No. 62/899,611, filed on Sep. 12, 2019.
(60) Provisional application No. 62/899,618, filed on Sep. 12, 2019.
(60) Provisional application No. 62/946,870, filed on Dec. 11, 2019.
(60) Provisional application No. 62/956,907, filed on Jan. 3, 2020.
(60) Provisional application No. 62/956,844, filed on Jan. 3, 2020.

(51) Int. Cl. A61K 47/54; A61K 31/661; A61K 31/688; A61P 35/00

(52) U.S. Cl. CPC A61K 47/544; A61K 31/661; A61K 31/688; A61P 35/00

(57) ABSTRACT

Disclosed herein are therapeutic compounds capable of targeting abroad range of tumor cells. The present disclosure is further directed to compositions comprising the therapeutic compounds, methods of manufacturing the therapeutic compounds, and methods of treating cancer comprising administering the therapeutic compounds.

### CROSS REFERENCE TO RELATED APPLICATIONS

This application claims priority to U.S. Provisional Pat. Application No. 62/899,611, filed Sep. 12, 2019, U.S. Provisional Pat. Application No. 62/899,615, filed Sep. 12, 2019, U.S. Provisional Pat. Application No. 62/899,618, filed Sep. 12, 2019, U.S. Provisional Pat. Application No. 62/946,870, filed Dec. 11, 2019, U.S. Provisional Pat. Application No. 62/956,844, filed Jan. 03, 2020, and U.S. Provisional Pat. Application No. 62/956,907, filed Jan. 03, 2020, the contents of which are incorporated herein by reference in their entirety.

### FIELD

This disclosure relates to therapeutic compounds capable of targeting a broad range of tumor cells. The present disclosure is further directed to compositions comprising the therapeutic compounds, methods of manufacturing the therapeutic compounds, and methods of treating cancer comprising administering the therapeutic compounds.

### INTRODUCTION

In 2018, 18 million people were diagnosed with cancer worldwide and 9.6 million died of cancer. In the United States, around 40% of all people will be diagnosed with cancer during their lifetime. As of 2018, lung cancer (2.09 million cases), breast cancer (2.09 million cases), colorectal cancer (1.80 million cases), prostate cancer (1.28 million cases), skin cancer (non-melanoma) (1.04 million cases), and stomach cancer (1.03 million cases) are the most common types of cancer. Despite many available treatments, cancer remains the second leading cause of death worldwide.

Cancer is the result of a cell dividing without limitation. Healthy cells have checkpoints that prevent unlimited cell division. A few examples of these checkpoints are nutrient availability, DNA damage and contact inhibition (i.e., a cell comes into contact with another cell). Additionally, most cells can replicate only a finite number of times and thus are programmed to die after a particular number of cell divisions.

Cancer is the result of a cell overcoming these built-in checkpoints and proliferating beyond control. This uncontrolled proliferation leads to the formation of a tumor. There are two types of tumors, benign and malignant. Benign tumors are incapable of crossing natural boundaries between tissue types. Malignant tumors, on the other hand, are capable of invading nearby tissue or entering the bloodstream and metastasizing to a different location. Only malignant tumors are considered cancerous. It is this ability to infiltrate and metastasize that makes cancer such a deadly disease. In addition, lipid metabolism may play a profound role in cancer metastasis. Cancer cells frequently display fundamentally altered cellular metabolism. However, the role of lipid metabolism in the development of malignant cancers remains obscure.

To further complicate the fight against cancer, malignant tumors have distinct cell types. One particularly troublesome type is cancer stem cells (“CSC’s”). CSC’s are capable of self-renewing and differentiating into the distinct types of cancer cells found in a malignant tumor. Thus, CSC’s are a primary factor in the metastatic ability of a tumor. CSC’s often survive radiation and chemotherapy. It is hypothesized that recurrence of cancer after radiation and chemotherapy is the result of the inability of radiation and chemotherapy to kill all CSC’s combined with the ability of CSC’s to establish a new tumor.

Chemotherapy is a term used to describe a particular type of cancer treatment that includes using cytotoxic anti-cancer drugs. Cytotoxic drugs used during chemotherapy can be broken down into several main categories including alkylating agents, antimetabolites, anti-tumor antibiotics, topoisomerase inhibitors, and mitotic inhibitors. Cytotoxic anti-cancer drugs typically cause cell division to cease and thus affect healthy tissue as well as cancerous tissue. Alkylating agents stop cancer cell division by damaging the DNA of the cancer cell. Some common alkylating agents used to treat cancer are nitrogen mustards (e.g. cyclophosphamide (Cytoxan®; Cytoxan is a registered trademark of Baxter International), nitrosoureas, alkyl sulfonates, triazeines, and ethylenimines. Platinum drugs, such as cisplatin and carboplatin, work similarly to alkylating agents. Antimetabolites stop cancer cell division by inhibiting DNA and RNA synthesis. Some common antimetabolites used to treat cancer are 6-mercaptopurine, gemcitabine (Gemzar®; Gemzar is a registered trademark of Eli Lilly and Company), methotrexate and pemetrexed (Alimta®; Alimta is a registered trademark of Eli Lilly and Company). Topoisomerase inhibitors stop cancer cell division by inhibiting topoisomerase enzymes from separating the DNA for replication. Some common topoisomerase inhibitors are topotecan, irinotecan, etoposide, and teniposide. Mitotic inhibitors stop cancer cell division by inhibiting key cell division enzymes. Some common mitotic inhibitors are taxanes (e.g. paclitaxel (Taxol®; Taxol is a registered trademark of Bristol-Myers Squibb Company) and docetaxel (Taxotere®; Taxotere is a registered trademark of Aventis Pharma SA)), epothilones, and vinca alkaloids.

One disadvantage of all of these anti-cancer drugs is the damage that they do to healthy tissue. Because the drugs treat cancer by inhibiting normal cell function, healthy tissue that also relies on constant cell division such as blood cells, mucosal surfaces and skin, can be severely damaged as well. This damage results in significant morbidity and can limit the amount of chemotherapy that can safely be delivered. Examples of side effects that occur during chemotherapy treatment include low blood count, hair loss, muscle, and joint pain, nausea, vomiting, diarrhea, mouth sores, fever, and chills. To overcome this problem, novel agents continue to be developed with unique mechanisms of action meant to provide increased targeting and that affect proteins and cellular functions that occur only in cancer cells. For example, antibody drug conjugates (ADCs) were designed to bind to specific epitopes on the surface of tumor cells and offer an alternative method to target tumor cells in an effort to reduce associated toxicities. Although highly selective, very few ADCs are therapeutically useful because they only achieve modest cellular uptake (<1% of infused drug) and have limited cell killing activity. Some specific cancer drugs are imatinib (Gleevec®; Gleevec is a registered trademark of Novartis AG), gefitinib (Iressa®, Iressa is a registered trademark of AstraZeneca UK Limited), sunitinib (Sutent®; Sutent is a registered trademark of C.P. Pharmaceuticals, International C.V.), and bortezomib (Velcade®; Velcade is a registered trademark of Millennium Pharmaceuticals, Inc.). However, these drugs are not approved for the treatment of all cancer types and are universally associated with the development of treatment resistance. In addition, many of these compounds still lack absolute tumor selectivity and continue to be limited in their therapeutic utilization due to off-target effects.

Recently, phospholipid ether (“PLE”) analogs were demonstrated to be an effective molecular platform for an anti-cancer drug delivery. See U.S. Pat. No. 9,480,754 and Weichert et al. (Sci Transl Med, 2014, 6(240), 240ra75), each of which are incorporated by reference herein in its entirety. As it can be seen, the majority of anticancer drugs in clinical use have limited utility due to their toxicity to all proliferating cells and/or the inability to exert their effect on all of the tumor cells. Thus, there remains a need in the art for alternative anti-cancer drug delivery vehicles that can deliver potent, effective, broad spectrum anti-cancer drugs to cancer cells including CSC’s while avoiding substantial uptake of the drug by healthy cells. Additionally, the anti-cancer drug delivery vehicle should be able to cross barriers such as the blood brain barrier (BBB).

### SUMMARY

In one aspect, the present disclosure provides a compound of formula (I), or a pharmaceutically acceptable salt thereof,

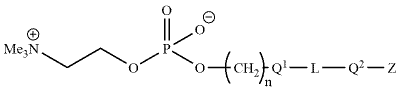

wherein n is 2-20; Q¹ is a bond or

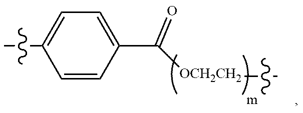

wherein m is 0-100; L is

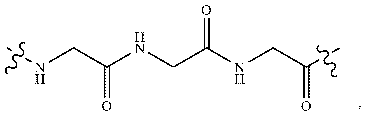

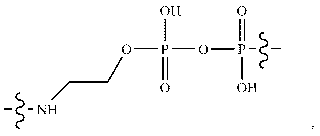

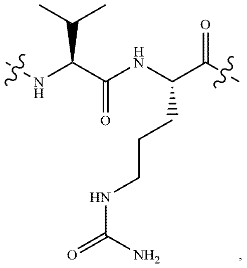

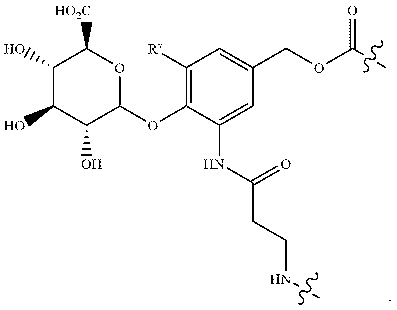

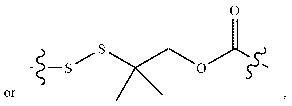

wherein R^{x} is H or halogen;

Q² is a bond or a self-immolative spacer; and

Z is an anti-cancer drug.

In another embodiment, the present disclosure provides a method of treating cancer in a subject in need thereof, comprising administering an effective amount of a compound as described herein, or a pharmaceutically acceptable salt thereof.

The disclosure provides for other aspects and embodiments that will be apparent in light of the following detailed description and accompanying drawings.

### BRIEF DESCRIPTION OF THE DRAWINGS

FIGS. 1A-1B show the uptake of phospholipid drug conjugates (PDCs) into tumor cell lines. FIG. 1A is green fluorescence is indicative of phospholipid ether (PLE) plus a BODIPY. FIG. 1B shows the ratio of MFI to autofluorescence ratio for CLR1502 uptake.

FIGS. 2A-2B show the uptake of PDCs into tumor cell lines (A375 and A549 cell lines). FIG. 2A shows the concentration of full conjugated PLE in the cytoplasm. FIG. 2B shows the concentration of payload released in the cytoplasm.

FIG. 3 shows the uptake of CLR1502 and CLR1501 via lipid rafts on tumor cells and primary tumor samples, respectively. CLR1502 is the near infrared molecule bound to the PLE and is white. Blue is the Hoechst nuclear stain. Red is cholera toxin subunit B and indicative of lipid rafts.

FIG. 4 shows the in vitro efficacy of PDC-SM2 against melanoma (A375) and lung cancer (A549) cells.

FIG. 5 shows cytotoxic PDCs that are tolerated in vivo. For the payload dose of 0.5 mg/kg, the circles indicate when mice died or were sacrificed. The arrows indicate when the doses were administered.

FIG. 6 shows in vitro uptake of CLR2000045 in MCF-7 and NHDF cell lines.

FIG. 7 shows in vitro cytotoxicity of CLR2000045 in breast cancer cell lines.

FIG. 8 shows in vivo antitumor activity in chicken embryo chorioallantoic membrane model (MCF-7).

FIG. 9 shows in vivo antitumor efficacy in implanted TNBC (HCC70) xenograft model.

FIG. 10 shows Kaplan-Meier survival curve in TNBC (HCC70) mouse xenograft model.

FIGS. 11A-11B show changes in body weight post treatment (HCC70) mouse xenograft model. FIG. 11A is 1 mg/kg administered 3x per week. FIG. 11B is 1 mg/kg administered 2x per week.

FIG. 12 shows in vitro uptake of CLR180099A and CLR180099B in A549 and NHDF cells.

FIG. 13 shows in vitro uptake of CLR180095 in A549 (black line) and HCT116 (grey line) cells. The cells were incubated over 48 hours and the initial incubation concentration was 100 nM. Uptake was assessed by LC/LC/MS.

FIG. 14 shows in vitro release of payload in A549 cells.

FIG. 15 shows in vitro cytotoxicity of CLR180099A in lung cancer, breast cancer and melanoma cells.

FIG. 16 shows in vivo antitumor efficacy of CLR180099A in an implanted colorectal cancer xenograft model.

FIG. 17 shows the Kaplan-Meier survival curve in the colorectal cancer xenograft model for CLR180099A.

FIG. 18 shows in vivo tolerability of CLR180099A.

FIGS. 19A-19F show the selective uptake of CLR1502 in intestinal tumors. FIG. 19A is the entire colon that was removed at necropsy 96 hours after administration of 50 µg of CLR1502 per mouse. FIG. 19B is the distal segment of the small intestine that was removed at necropsy 96 hours after administration of 50 µg of CLR1502 per mouse. Areas of increased signal intensity were observed using the IVIS Spectrum. These areas non-invasive (colon FIG. 19C; distal small intestine FIG. 19F) and invasive (colon FIG. 19D; distal small intestine FIG. 19E) tumors. FIG. 19C, FIG. 19D, FIG. 19E, and FIG. 19F are magnified as shown by the black box. Arrows point to malignant glands within the intestinal musculature. Bars: 1 mm.

FIGS. 20A-20H shows uptake of CLR1501 in the brain. FIG. 20C, FIG. 20D, and FIG. 20E show a U251-derived orthotopic brain tumor verified by magnetic resonance imaging (MRI; FIG. 20C, T2-weighted) and labeled with CLR1501 (FIG. 20E) with ToPro3 nuclear counterstain (FIG. 20D). FIG. 20F, FIG. 20G, and FIG. 20H are histological analyses of brain-tumor interface in 22T glioblastoma multiforme-derived orthotopic xenograft labeled with CLR1501 (green). FIG. 20F is an epifluorescent visulaization of the xenograft-brain border with blue DAPI nuclear counterstain. FIG. 20G is a confocal view of a xenograft labeled with CLR1501. FIG. 20H is a confocal and bright-field view of xenograft and adjacent normal brain. N indicates normal brain; RFU indicates relative fluorescent units; T indicates tumor.

FIG. 21A shows CLR1502 treated brain in vivo with visible light (left) and CLR1502 fluorescence of 22CSC-derived orthotopic xenograft in vivo (right).

FIG. 21B shows CLR1502 treated brain and tumor ex vivo with visible light (upper left) and CLR1502 fluorescence of 22CSC-derived xenograft ex vivo demonstrating excellent macroscopic tumor delineation from normal brain (upper right). The figure also shows the verification of tumor (T) by histology (hematoxylin and eosin; lower left) and the verification of normal brain (N) by histology (hematoxylin and eosin; lower right).

FIG. 22 shows that tumor thickness does not account for the increased signal intensity noted in the intestinal cancers. FIG. 22A shows layers of the colon. FIG. 22B shows the total radiant efficiency for each layer.

FIG. 23 shows in vivo optical scanning of CLR1502 uptake in a colorectal carcinoma model. Fluorescence intensity (indicated by color bar) and biodistribution were determined in vivo over time.

FIG. 24 shows in vivo optical scanning of CLR1502 uptake in a breast cancer model. An athymic nude mouse bearing an orthotopic breast cancer xenograft (MDA-MB-231) was imaged daily for seven days (168 hr) using Fluoptics Fluobeam® and IVIS® Spectrum systems (yellow and green arrows for Fluobeam and IVIS Spectrum, respectively).

FIG. 25 shows an athymic nude mouse bearing a lung cancer xenograft (H226 lung) on each flank injected intravenously with CLR1502 was imaged using the IVIS Spectrum. At 96 hours the difference in radiant efficiency between the malignant and normal tissue creates sufficient contrast for tumor margin illumination, as indicated by black arrows.

### DETAILED DESCRIPTION

Described herein are therapeutic compounds capable of targeting a broad range of tumor cells. The compounds disclosed herein may target specialized structures in tumor cell membranes such as lipid rafts. Accordingly, the compounds disclosed herein may be used to target tumor cells with high specificity. In particular, the compounds disclosed herein may be used for the treatment of cancer.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March’s Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

As used herein the term “cancer” refers to any disease that results from the uncontrolled division of cells capable of metastasizing. The term “cancer”, as used herein, refers to, but is not limited to, a variety of cancer types including breast cancer including male breast cancer; digestive/gastrointestinal cancers including anal cancer, appendix cancer, extrahepatic bile duct cancer, gastrointestinal carcinoid tumor, colon cancer, esophageal cancer, gallbladder cancer, gastric cancer, gastrointestinal stromal tumors (“gist”), Islet cell tumors, adult primary liver cancer, childhood liver cancer, pancreatic cancer, rectal cancer, small intestine cancer, and stomach (gastric) cancer; endocrine and neuroendocrine cancers including pancreatic adenocarcinoma, adrenocortical carcinoma, pancreatic neuroendocrine tumors, Merkel cell carcinoma, non- small cell lung neuroendocrine tumor, small cell lung neuroendocrine tumor, parathyroid cancer, pheochromocytoma, pituitary tumor and thyroid cancer; eye cancers including intraocular melanoma and retinoblastoma; genitourinary cancer including bladder cancer, kidney (renal cell) cancer, penile cancer, prostate cancer, transitional cell renal pelvis and ureter cancer, testicular cancer, urethral cancer and Wilms tumor; germ cell cancers including childhood central nervous system cancer, childhood extracranial germ cell tumor, extragonadal germ cell tumor, ovarian germ cell tumor and testicular cancer; gynecologic cancers including cervical cancer, endometrial cancer, gestational trophoblastic tumor, ovarian epithelial cancer, ovarian germ cell tumor, uterine sarcoma, vaginal cancer and vulvar cancer; head and neck cancers including hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, parathyroid cancer, pharyngeal cancer, salivary gland cancer and throat cancer; leukemias including adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; multiple myeloma including malignant plasma cells; lymphomas including AIDS-related lymphoma, cutaneous t-cell lymphoma, adult Hodgkin lymphoma, childhood Hodgkin lymphoma, Hodgkin lymphoma during pregnancy, mycosis fungoides, adult non-Hodgkin lymphoma, childhood non- Hodgkin lymphoma, non-Hodgkin lymphoma during pregnancy, primary central nervous system lymphoma, Sezary syndrome and Waldenstrom macroglobulinemia; musculoskeletal cancers including Ewing sarcoma, osteosarcoma and malignant fibrous histocytoma of bone, childhood rhabdomyosarcoma and soft-tissue sarcoma; neurological cancers including adult brain tumor, childhood brain tumor, astrocytomas, brain stem glioma, central nervous system atypical teratoid/rhabdoid tumor, central nervous system embryonal tumors, craniopharyngioma, ependymoma, neuroblastoma, primary central nervous system (CNS) lymphoma; respiratory/thoracic cancers including non-small cell lung cancer, small cell lung cancer, malignant mesothelioma, thymoma and thymic carcinoma; and skin cancers including Kaposi sarcoma, melanoma and squamous cell carcinoma.

As used herein the term “cancer stem cell” refers to a cancer cell capable of self-renewing and differentiating into the distinct types of cancer cells found in a malignant tumor.

The terms “chemotherapy drug” “anti-cancer drug” and “anti-tumor drug” are used interchangeably throughout the specification.

In general, reference to “a circulating tumor cell” is intended to refer to a single cell, while reference to “circulating tumor cells” or “cluster of circulating tumor cells” is intended to refer to more than one cancer cell. However, one of skill in the art would understand that reference to “circulating tumor cells” is intended to include a population of circulating tumor cells including one or more circulating tumor cells while reference to “a circulating tumor cell” could include more than one circulating tumor cell. The term “circulating tumor cell” or “circulating tumor cells”, as used herein, refers to any cancer cell or cluster of cancer cells that are found in a subject’s blood or blood serum sample. CTCs may also contain or consist of a cancer stem cell or cluster of cancer stem cells that are found in a subject’s blood or blood serum sample.

As used herein, the term “composition” is intended to encompass a product comprising the specified ingredients in the specified amounts, as well as any product which results, directly or indirectly, from a combination of the specified ingredients in the specified amounts.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying an ideal patient to receive an IL-1 Ra therapy. A description of ROC analysis is provided in P.J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without an tumor as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

The term “dose” as used herein denotes any form of the active ingredient formulation or composition that contains an amount sufficient to produce a therapeutic effect with at least a single administration. “Formulation” and “compound” are used interchangeably herein.

The term “dosage” as used herein refers to the administering of any amount, number, and frequency of doses over a specified period of time.

The terms “effective amount” or “therapeutically effective amount,” as used herein, refer to any amount of an agent or pharmaceutically acceptable composition or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “halogen” as used herein, means Cl, Br, I, F, At, or synthetic halogens such as tennessine (Ts).

As used herein the term “heterocycloalkyl” refers to a cyclic group of 3 to 24 atoms (C3-C24) selected from carbon, nitrogen, sulfur, phosphate and oxygen wherein at least one atom is carbon.

As defined herein, the term “isomer” includes, but is not limited to optical isomers and analogs, structural isomers and analogs, conformational isomers and analogs, and the like. In one embodiment, this disclosure encompasses the use of different optical isomers as detailed herein. It will be appreciated by those skilled in the art that the anti-cancer compounds useful in the present invention may contain at least one steriogenic center. Accordingly, the compounds used in the methods of the present invention may exist in, and be isolated in, optically-active or racemic forms. Some compounds may also exhibit polymorphism.

The term “malignant tumor cell,” “tumor cell,” and “cancer cell” are used interchangeably throughout the specification. The term “malignant tumor stem cell,” “tumor stem cell,” and “cancer stem cell” are used interchangeably throughout the specification.

“Sample” or “test sample” as used herein can mean any sample in which the presence and/or level of a target is to be detected or determined. Samples may include liquids, solutions, emulsions, or suspensions. Samples may include a medical sample. Samples may include any biological fluid or tissue, such as blood, whole blood, fractions of blood such as plasma and serum, cartilage, ligaments, tendons, muscle, interstitial fluid, sweat, saliva, urine, tears, synovial fluid, synovial membrane, meniscus, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pretreated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific cancer. The subject may be undergoing other forms of treatment.

As used herein the term “therapeutic compound” refers to any chemical compound capable of providing treatment for cancer.

“Treat” or “treating” or “treatment” means suppressing, repressing, reversing, alleviating, ameliorating, or inhibiting the deterioration of a disease, or completely eliminating the disease. A treatment may be either performed in an acute or chronic way. The term also refers to reducing the severity of a disease or symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Compounds

In one aspect, the present disclosure provides a compound of formula (I), or a pharmaceutically acceptable salt thereof,

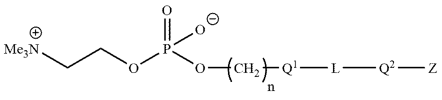

wherein n is 2-20;

Q¹ is a bond or

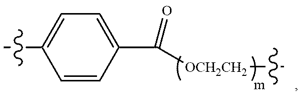

wherein m is 0-100;

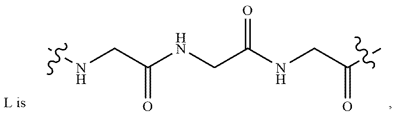

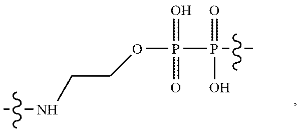

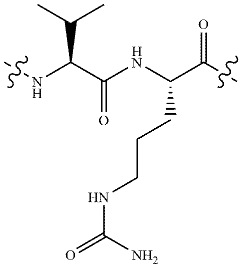

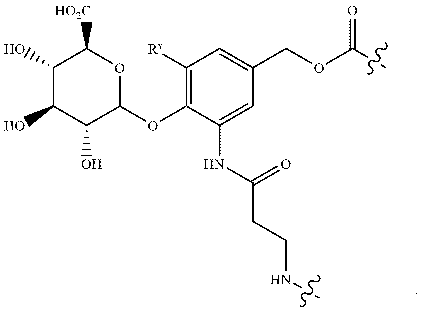

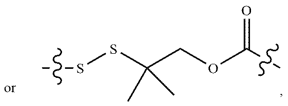

wherein R^{x} is H or halogen; Q² is a bond or a self-immolative spacer; and

Z is an anti-cancer drug.

The number “n” may be any integer from 2 to 20. In some embodiments, n is 2, 4, 6, 8, 10, 12, 14, 16, 18, or 20. In particular embodiments, n is 18.

The number “m” may be any integer from 0 to 100. In some embodiments, m is 0, 1, 2, 3, 4, 5, 6, 7, 8, 9, or 10. In some embodiments, m is an integer from 10 to 20, from 10 to 40, from 10 to 60, or from 10 to 80. In some embodiments, m is 0, and Q¹ is a bond or

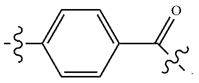

Q² may be any known self-immolative spacer, including, for example, para-aminobenzyloxycarbonyl (PABC).

In some embodiments, R^{X} is H. In some embodiments, R^{X} is Cl.

In some embodiments, n is 2-20, Q¹ is a bond or

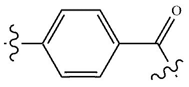

and the L-Q² moiety

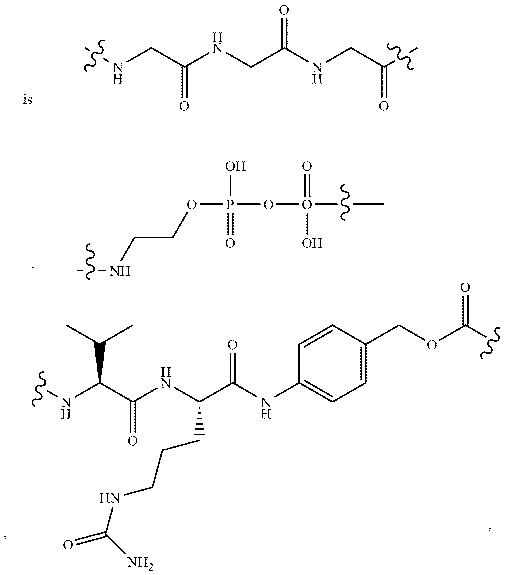

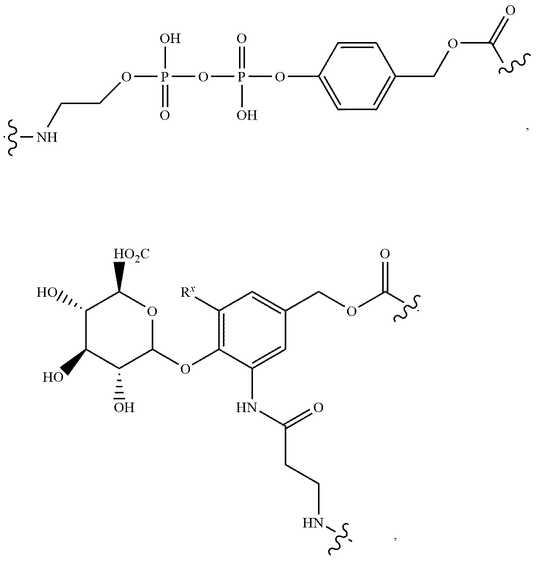

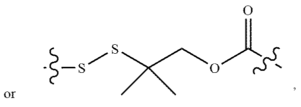

R^{x} is H or halogen, and Z is an anti-cancer drug.

Z may be any anti-cancer drug, including various known chemotherapy drugs.

In some embodiments, Z is a polo-like kinase 1 (PLK-1) inhibitor. Suitable PLK-1 inhibitors include, for example, BI2536, BI6727 (volasertib), diaminopyrimidine (DAP) derivatives such as DAP-81 and DAP-83, as well as the compounds disclosed in Kumar et al. (Biomed Res Int. 2015, 2015: 705745) and Peters et al. (Nat Chem Biol. 2006, 2(11):618-26), the contents of which are incorporated herein by reference in their entireties.

In some embodiments, Z is a tubulin polymerase inhibitor, such as nocodazole.

In some embodiments, Z is a tubulin stabilizer, such as taccalonolides.

In some embodiments, Z is an antineoplastic agent, such as monomethyl auristatin E (MMAE), monomethyl auristatin F (MMAF), monomethyl auristatin D (MMAD).

In some embodiments, Z is an eukaryotic translation initiation factor 4 (EIF4) inhibitor, such as EIF4A and EIF4E inhibitors. In some embodiments, Z is an EIF4E inhibitor. Suitable EIF4 inhibitors include, for examples, ribavirin and the compounds disclosed in D’Abronzo et al. (Neoplasia, 2018, 20(6), 563-573) and U.S. Pat. No. 10,577,378, the contents of which are incorporated herein by reference in their entireties.

In some embodiments, Z is a combretastatin A-4 analog, such as combretastatin A-4 phosphate or ombrabulin. Suitable combretastatin A-4 analogs also include, for example the compounds disclosed in Bellina et al. (Bioorganic & Medicinal Chemistry Letters 2006, 16(22), 5757-5762), the content of which is incorporated herein by reference in its entirety.

In some embodiments, Z is flavagline analog. Suitable flavagline analogs include, for example, the compounds disclosed in U.S. Pat. Application Publication US 2018/0086729, the content of which is incorporated herein by reference in its entirety.

In certain embodiments, Z is one of other known anti-cancer drugs, including for example, (i) other antiproliferative/antineoplastic drugs, such as alkylating agents, antimetabolites, antitumor antibiotics, antimitotic agents; and topoisomerase inhibitors; (ii) cytostatic agents such as antioestrogens, antiandrogens, LHRH antagonists or LHRH agonists, progestogens, and aromatase inhibitors; (iii) anti-invasion agents (for example c-Src kinase family inhibitors); (iv) inhibitors of growth factor function, such as tyrosine kinase inhibitors; (v) antiangiogenic agents; (vi) vascular damaging agents; and (vii) endothelin receptor antagonists.

Examples of suitable anti-cancer drugs include, but are not limited to, paclitaxel, irinotecan, topotecan, gemcitabine, cisplatin, geldanamycin, mertansine, abiraterone, afatinib, aminolevulinic acid, aprepitant, axitinib, azacitidine, belinostat, bendamustine, bexarotene, bleomycin, bortezomib, bosutinib, busulfan, cabazitaxel, cabozantinib, capecitabine, carboplatin, carfilzomib, carmustine, ceritinib, cetuximab, chlorambucil, clofarabine, crizotinib, cyclophosphamide, cytarabine, dabrafenib, dacarbazine, dactinomycin, dasatinib, daunorubicin, decitabine, denosumab, dexrazoxane, docetaxel, dolastatins (e.g. monomethyl auristatin E), doxorubicin, enzalutamide, epirubicin, eribulin mesylate, erlotinib, etoposide, everolimus, floxuridine, fludarabine phosphate, fluorouracil, ganetespib, gefitinib, gemtuzumab ozogamicin, hexamethylmelamine, hydroxyurea, ibritumomab tiuxetan, ibrutinib, idelalisib, ifosfamide, imatinib, ipilimumab, ixabepilone, lapatinib, leucovorin calcium, lomustine, maytansinoids, mechlorethamine, melphalan, mercaptopurine, mesna, methotrexate, mitomycin C, mitotane, mitoxantrone, nelarabine, nelfinavir, nilotinib, obinutuzumab, ofatumumab, omacetaxine mepesuccinate, oxaliplatin, panitumumab, pazopanib, pegaspargase, pembrolizumab, pemetrexed, pentostatin, pertuzumab, plicanycin, pomalidomide, ponatinib hydrochloride, pralatrexate, procarbazine, radium 223 dichloride, ramucirumab, regorafenib, retaspimycin, ruxolitinib, semustine, siltuximab, sorafenib, streptozocin, sunitinib malate, tanespimycin, temozolomide, temsirolimus, teniposide, thalidomide, thioguanine, thiotepa, toremifene, trametinib, trastuzumab, vandetanib, vemurafenib, vinblastine, vincristine, vinorelbine, vismodegib, vorinostat, and ziv-aflibercept.

In some embodiments, the compounds of formula (I) has a structure of formula (I-a), or a pharmaceutically acceptable salt thereof, wherein Q¹ is

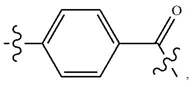

L-Q² is

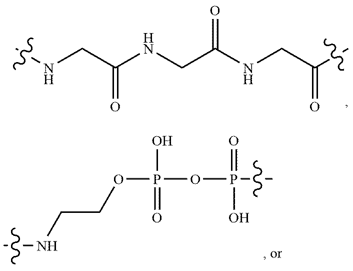

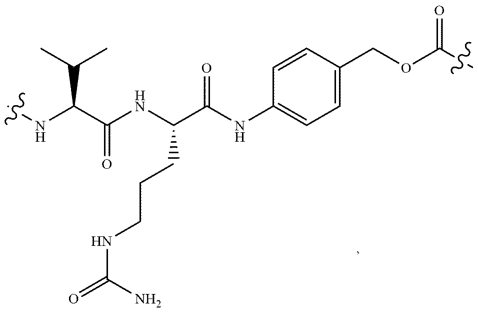

and Z is a PLK-1 inhibitor, a tubulin polymerase inhibitor, a tubulin stabilizer, an antineoplastic agent, or an eukaryotic translation initiation factor 4 (EIF4) inhibitor. Specifically, formula (I-a) may be

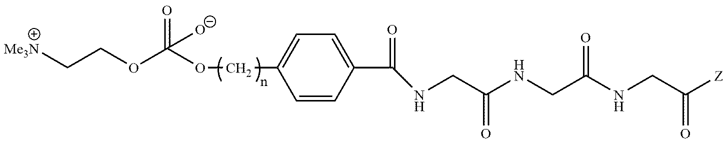

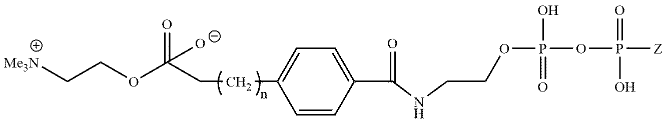

, or

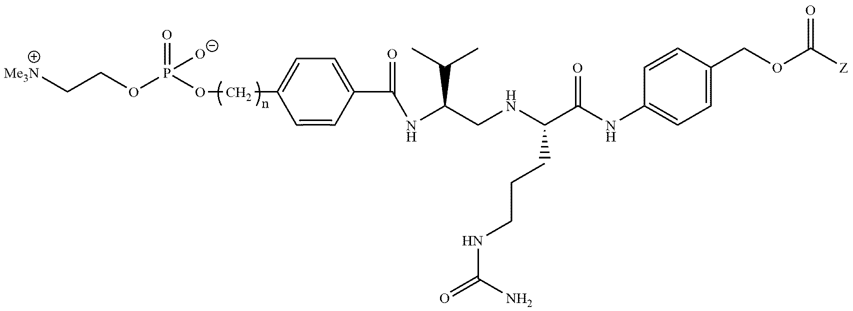

or a pharmaceutically acceptable salt thereof, wherein n is 2-20 and Z is a PLK-1 inhibitor, a tubulin polymerase inhibitor, a tubulin stabilizer, an antineoplastic agent, or an eukaryotic translation initiation factor 4 (EIF4) inhibitor.

In some embodiments, the compound has a structure of formula (I-a), wherein n is 18. In some embodiments, the compound has a structure of formula (I-a), wherein Z is a PLK-1 inhibitor or an antineoplastic agent. In some embodiments, the compound has a structure of formula (l-a-1), (l-a-2), or (l-a-3),or a pharmaceutically acceptable salt thereof wherein Z is PLK-1 inhibitor. For example, Z may be

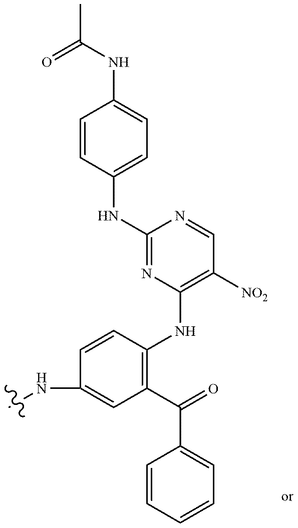

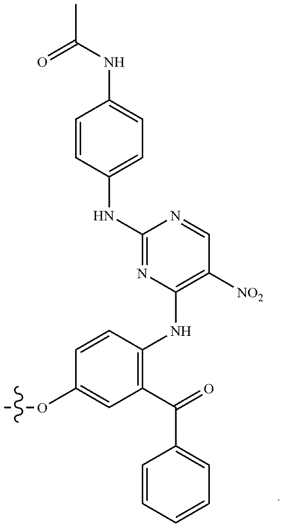

In some embodiments, the compound has a structure of formula (l-a-1), (l-a-2), or (l-a-3), or a pharmaceutically acceptable salt thereof wherein Z is an antineoplastic agent selected from the group consisting of monomethyl auristatin E (MMAE), monomethyl auristatin F (MMAF), and monomethyl auristatin E (MMAD). In some embodiments, the compound has a structure of formula (l-a-3),or a pharmaceutically acceptable salt thereof wherein Z is MMAE, MMAF, or MMAD (shown below).

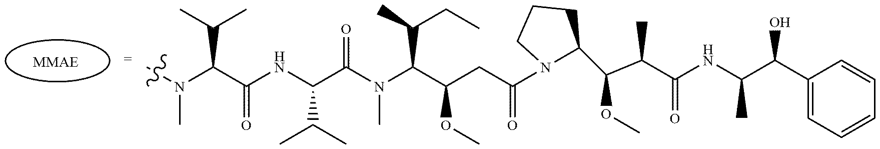

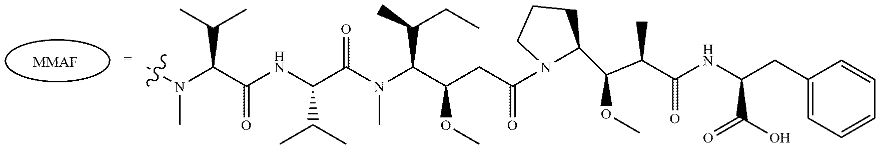

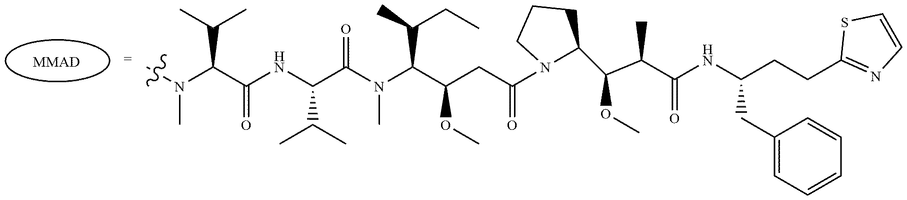

In some embodiments, the compounds of formula (I) has a structure of formula (l-b), or a pharmaceutically acceptable salt thereof, wherein n is 18, Q¹ is

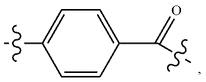

L-Q² is

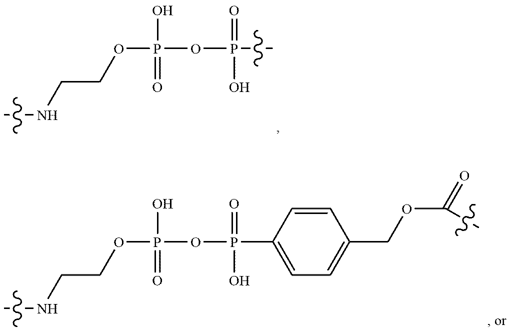

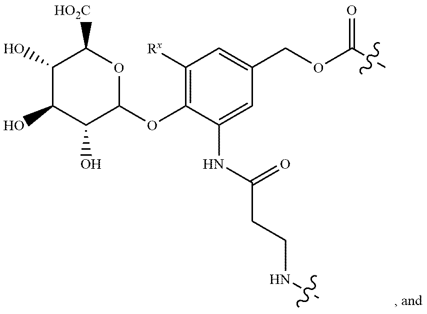

Z is a combretastatin A-4 analog. Specifically, formula (l-b) may be

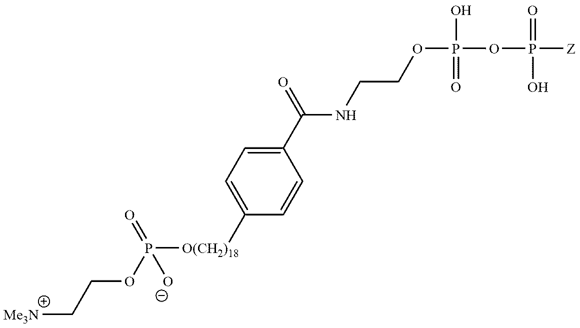

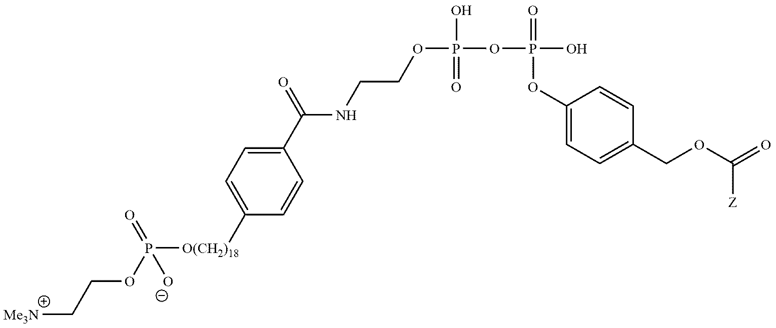

or

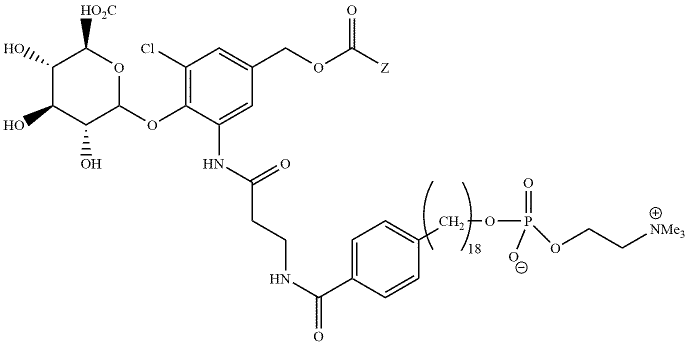

or a pharmaceutically acceptable salt thereof, wherein Z is a combretastatin A-4 analog.

In some embodiments, the compound has a structure of formula (l-b-1),(l-b-2),or (l-b-3), or a pharmaceutically acceptable salt thereof, wherein Z is a combretastatin A-4 analog, such as

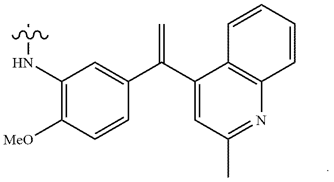

In some embodiments, the compounds of formula (I) has a structure of formula (l-c), or a pharmaceutically acceptable salt thereof, wherein n is 18, Q¹ is a bond or

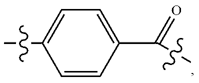

L-Q² is

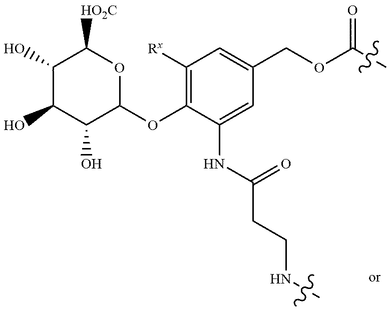

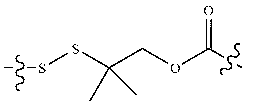

and Z is a flavagline analog. Specifically, formula (l-c) may be

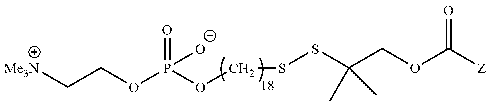

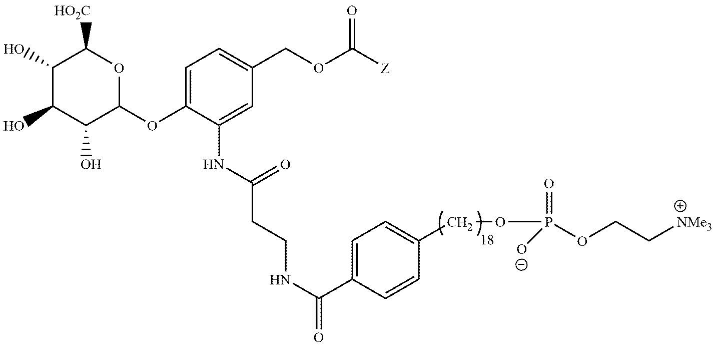

or

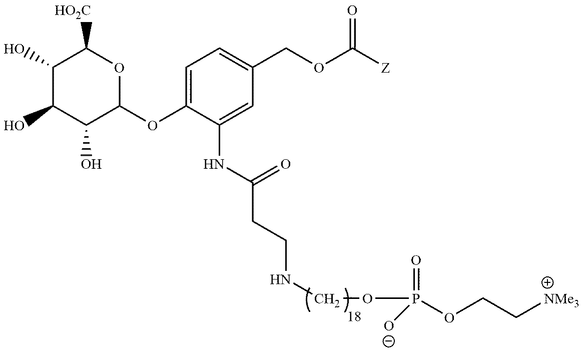

or a pharmaceutically acceptable salt thereof, wherein Z is a flavagline analog.

In some embodiments, the compound has a structure of formula (l-c-1), (l-c-2), or (l-c-3), or a pharmaceutically acceptable salt thereof, wherein Z is a flavagline analog, such as

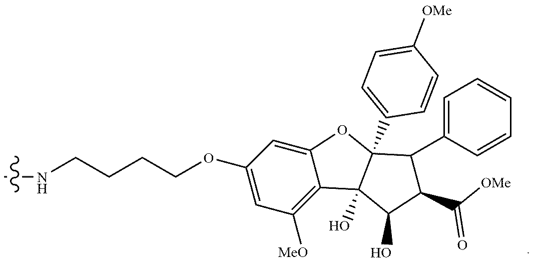

Suitable compounds as disclosed herein include:

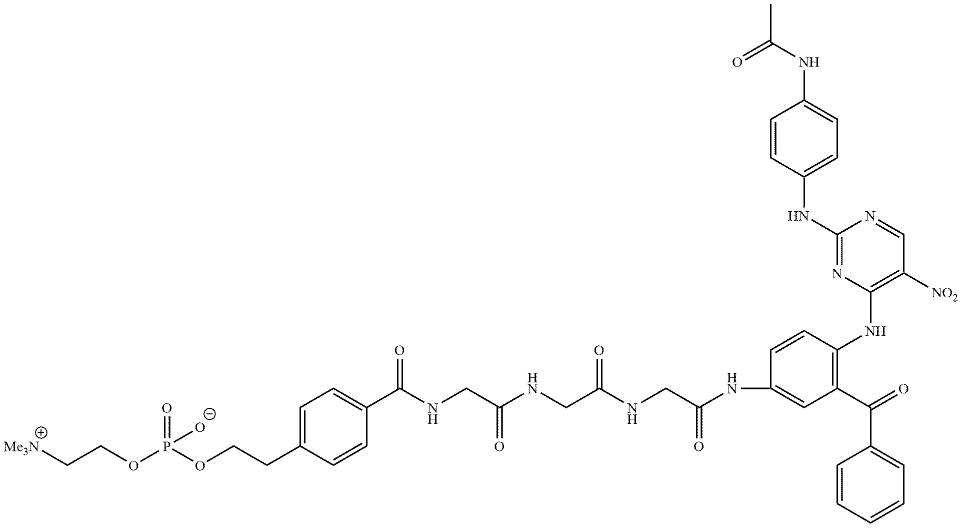

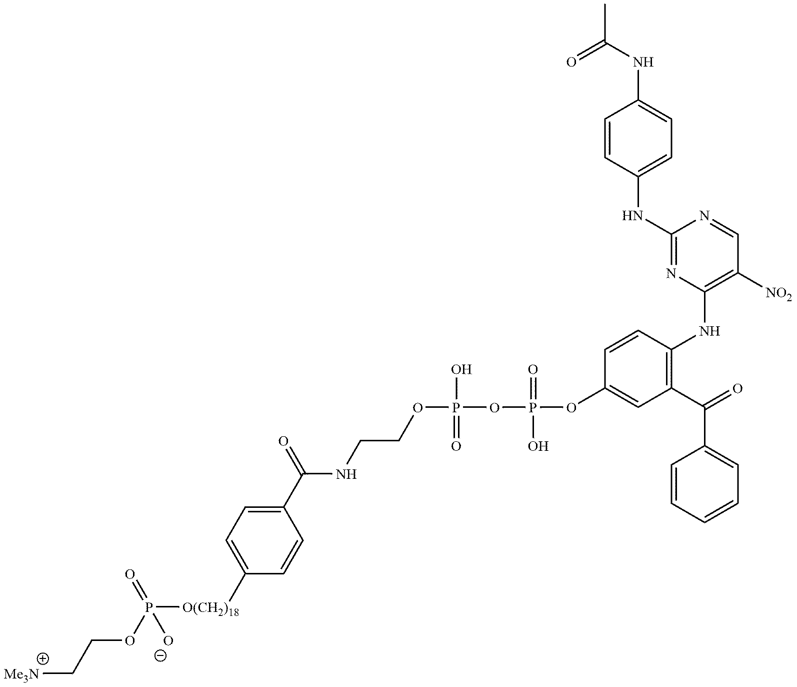

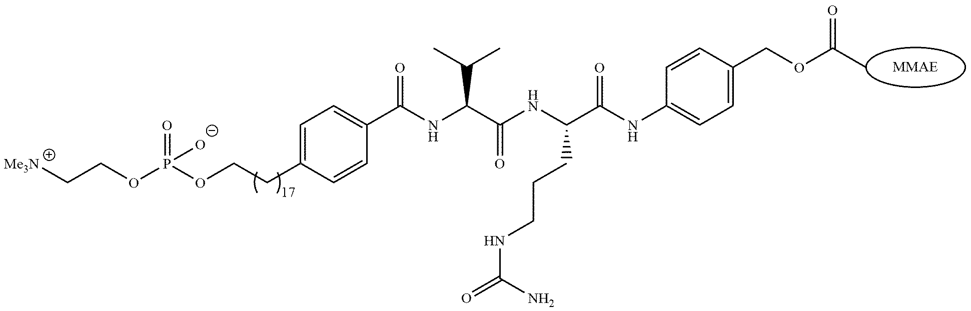

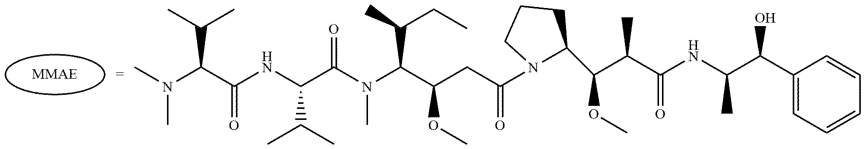

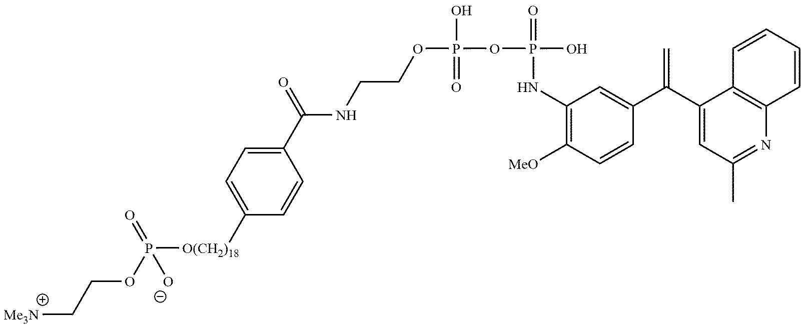

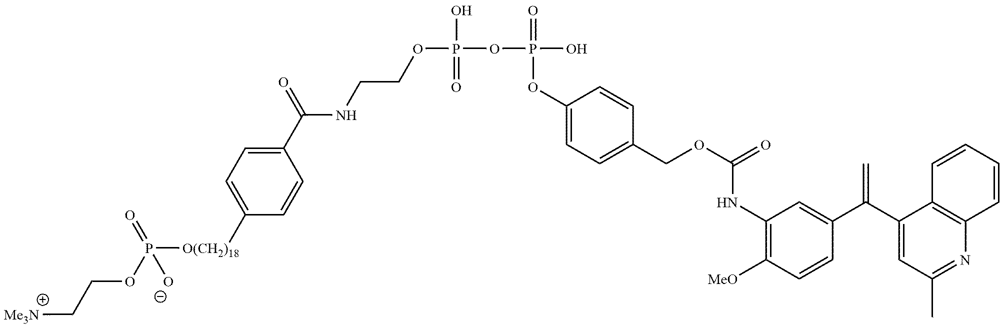

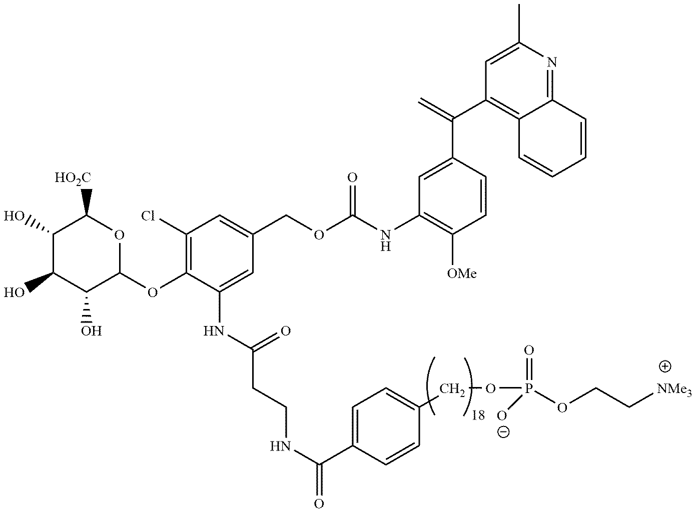

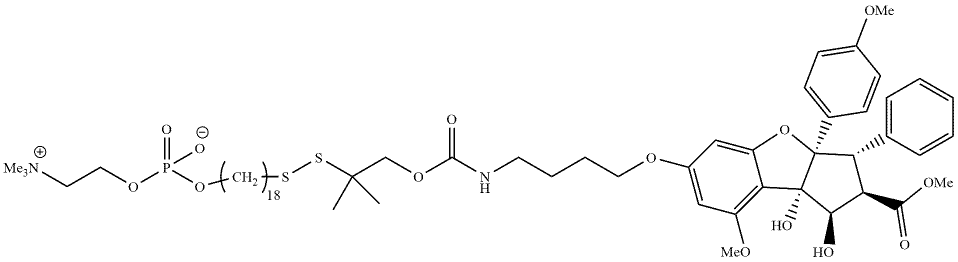

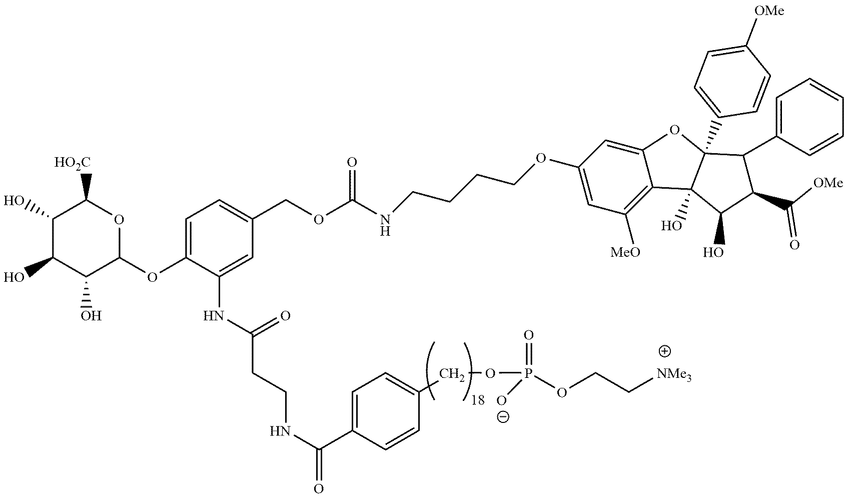

and

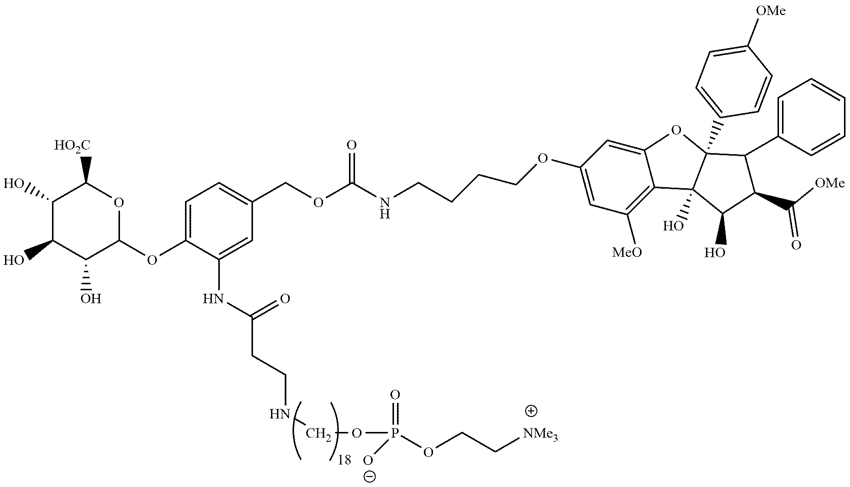

or a pharmaceutically acceptable salt thereof.

The disclosed compounds may exist as pharmaceutically acceptable salts. The term “pharmaceutically acceptable salt” refers to salts or zwitterions of a compounds which are water or oil-soluble or dispersible, suitable for treatment of disorders without undue toxicity, irritation, and allergic response, commensurate with a reasonable benefit/risk ratio and effective for their intended use. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl, and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, Nmethylpiperidine, N-methylmorpholine, dicyclohexylamine, procaine, dibenzylamine, N,Ndibenzylphenethylamine, 1-ephenamine and N,N′-dibenzylethylenediamine, ethylenediamine, ethanolamine, diethanolamine, piperidine, piperazine, and the like.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel’s Textbook of Practical Organic Chemistry,” 5th edition (1989), Longman Scientific & Technical, Essex CM20 2JE, England, or (2) direct separation of the mixture of optical enantiomers on chiral chromatographic columns or (3) fractional recrystallization methods. It should be understood that the compound may possess tautomeric forms, as well as geometric isomers, and that these also constitute an aspect of the present disclosure.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the disclosure are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positronemitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

The compounds may be prepared by the synthesis schemes detailed herein. The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel’s Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene’s book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes and in the specific examples.

### 3. Pharmaceutical Compositions

In another aspect, the present disclosure provides a pharmaceutical composition comprising a compound as disclosed herein, or a pharmaceutically acceptable salt thereof, and a pharmaceutically acceptable carrier.

The present pharmaceutical compositions may be manufactured by processes known in the art, e.g., by means of conventional mixing, dissolving, granulating, dragee-making, levigating, emulsifying, encapsulating, entrapping or lyophilizing processes.

As described herein, the pharmaceutically acceptable carrier includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Various carriers used in formulating pharmaceutically acceptable compositions and techniques for the preparation thereof are known in the art (e.g., Remington’s Pharmaceutical Sciences, Sixteenth Edition, E.W. Martin (Mack Publishing Co., Easton, Pa., 1980)).

The pharmaceutically acceptable carrier may be a functional molecule such as a vehicle, an adjuvant, or diluent. The pharmaceutically acceptable carrier may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof.

Some examples of materials which can serve as pharmaceutically acceptable carriers include, but are not limited to, ion exchangers, alumina, aluminum stearate, lecithin, serum proteins (such as human serum albumin), buffer substances (such as phosphates), glycine, sorbic acid, or potassium sorbate, partial glyceride mixtures of saturated vegetable fatty acids, water, salts or electrolytes (such as protamine sulfate, disodium hydrogen phosphate, potassium hydrogen phosphate, sodium chloride, zinc salts), colloidal silica, magnesium trisilicate, polyvinyl pyrrolidone, polyacrylates, waxes, polyethylenepolyoxypropylene-block polymers, wool fat, sugars (such as lactose, glucose, and sucrose), starches (such as com starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil, soybean oil), glycols (such a propylene glycol or polyethylene glycol), esters (such as ethyl oleate and ethyl laurate), agar, non-toxic compatible lubricants (such as sodium lauryl sulfate and magnesium stearate), coloring agents, releasing agents, coating agents, emulsifying agents, sweetening, flavorant, perfuming agents, preservatives, antioxidants can also be present in the composition, according to the judgment of the formulator.

In some embodiments, the pharmaceutical composition consists essentially of a therapeutically effective amount of a compound as disclosed herein, or a pharmaceutically acceptable salt thereof.

Liquid dosage forms include, but are not limited to, pharmaceutically acceptable emulsions, microemulsions, solutions, suspensions, syrups and elixirs. Solid dosage forms include, but are not limited to, capsules, tablets, pills, powders, cement, putty, and granules. Dosage forms for topical or transdermal administration of the present compounds include, but are not limited to, ointments, pastes, creams, lotions, gels, powders, solutions, sprays, inhalants or patches.

A liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

The pharmaceutical composition may be in a dosage form suitable for injection or infusion, such as sterile aqueous solutions or dispersions or sterile powders comprising the active ingredient(s) which are adapted for the extemporaneous preparation of sterile injectable or infusible solutions or dispersions. The ultimate dosage form should be sterile, fluid and stable under manufacture and storage conditions. Sterile injectable solutions may be prepared by incorporating at least a compound as disclosed herein, or a pharmaceutically acceptable salt thereof in the required amount in the appropriate solvent with various other ingredients, as required, optionally followed by filter sterilization. In the case of sterile powders for the preparation of sterile injectable solutions, the methods of preparation may include vacuum drying and freeze-drying techniques, which yield a powder of the active ingredient(s) plus any additional desired ingredient present in the sterile solutions.

In some embodiments, the composition is a solution, such as a solution suitable for administration by infusion or injection. Solutions may be prepared in water, optionally mixed with a nontoxic surfactant. Dispersions may also be prepared in glycerol, liquid polyethylene glycols, triacetin, and mixtures thereof and in oils. These preparations may contain a preservative to prevent the growth of microorganisms. Prevention of the action of microorganisms can be brought about by various antibacterial and antifungal agents, for example, parabens, chlorobutanol, phenol, sorbic acid, thimerosal, and the like.

Injectable forms may be made by forming microencapsule matrices of the compound(s) as disclosed herein, or pharmaceutically acceptable salt thereof, in biodegradable polymers such as polylactide-polyglycolide. Depending upon the ratio of compound to polymer and the nature of the particular polymer employed, the rate of drug release can be controlled. Examples of other biodegradable polymers include poly(orthoesters) and poly(anhydrides). Injectable formulations are also prepared by entrapping the drug in liposomes or microemulsions which are compatible with body tissues.

In some embodiments, the composition may comprise at least one compound as described herein and at least one additional anti-cancer drug. Anti-cancer drugs that are useful for the present disclosure include, but are not limited to, paclitaxel, irinotecan, topotecan, gemcitabine, cisplatin, geldanamycin, mertansine, abiraterone, afatinib, aminolevulinic acid, aprepitant, axitinib, azacitidine, belinostat, bendamustine, bexarotene, bleomycin, bortezomib, bosutinib, busulfan, cabazitaxel, cabozantinib, capecitabine, carboplatin, carfilzomib, carmustine, ceritinib, cetuximab, chlorambucil, clofarabine, crizotinib, cyclophosphamide, cytarabine, dabrafenib, dacarbazine, dactinomycin, dasatinib, daunorubicin, decitabine, denosumab, dexrazoxane, docetaxel, dolastatins (e.g. monomethyl auristatin E), doxorubicin, enzalutamide, epirubicin, eribulin mesylate, erlotinib, etoposide, everolimus, floxuridine, fludarabine phosphate, fluorouracil, ganetespib, gefitinib, gemtuzumab ozogamicin, hexamethylmelamine, hydroxyurea, ibritumomab tiuxetan, ibrutinib, idelalisib, ifosfamide, imatinib, ipilimumab, ixabepilone, lapatinib, leucovorin calcium, lomustine, maytansinoids, mechlorethamine, melphalan, mercaptopurine, mesna, methotrexate, mitomycin C, mitotane, mitoxantrone, nelarabine, nelfinavir, nilotinib, obinutuzumab, ofatumumab, omacetaxine mepesuccinate, oxaliplatin, panitumumab, pazopanib, pegaspargase, pembrolizumab, pemetrexed, pentostatin, pertuzumab, plicanycin, pomalidomide, ponatinib hydrochloride, pralatrexate, procarbazine, radium 223 dichloride, ramucirumab, regorafenib, retaspimycin, ruxolitinib, semustine, siltuximab, sorafenib, streptozocin, sunitinib malate, tanespimycin, temozolomide, temsirolimus, teniposide, thalidomide, thioguanine, thiotepa, toremifene, trametinib, trastuzumab, vandetanib, vemurafenib, vinblastine, vincristine, vinorelbine, vismodegib, vorinostat, and ziv-aflibercept. Any compounds that are currently known to or are capable of acting as anti-cancer drugs are also useful for the present disclosure.

### 4. Methods

The basis for selective tumor targeting of the compounds detailed herein lies in differences between the plasma membranes of cancer cells as compared to those of most normal cells. Phospholipid ether (PLE) molecules take advantage of the metabolic shift that tumors cells undergo in order to generate the energy necessary for the rapid cell division. Tumors enhance the utilization of the beta oxidative pathway to convert long chain fatty acids (LCFA) into energy. In order to increase the uptake of LCFA, tumor cells alter the cell membrane forming specialized microdomains known as “lipid rafts.” Lipid rafts form due to metabolic shifts and need for phospholipids. Within tumor cells these regions have become overabundant and stabilized allowing them to be potential tumor specific targets. Specifically, cancer cell membranes are highly enriched in lipid rafts. In normal tissue the presence of lipid rafts is limited and transient (~2 nanoseconds). In tumors, lipid rafts have increased presence and are stabilized (up to 10 days). Cancer cells have five to ten times more lipid rafts than healthy cells. In addition, lipid rafts have been demonstrated to be highly abundant on nearly all tumor types and 100% of individual cancer cells tested. Lipid rafts are highly organized and specialized regions of the membrane phospholipid bilayer, that contain high concentrations of various signaling molecules, sphingolipids, glycosphingolipids and cholesterol, and serve to organize cell surface and intracellular signaling molecules (e.g., growth factor and cytokine receptors, the phosphatidylinositol 3-kinase (Pl3K)/Akt survival pathway). Data suggests that lipid rafts serve as portals of entry for phospholipid ethers (PLEs). The marked selectivity of these compounds for cancer cells versus non-cancer cells is attributed to the high affinity of PLEs for cholesterol and the abundance of cholesterol-rich lipid rafts in cancer cells. The pivotal role played by lipid rafts is underscored by the fact that disruption of lipid raft architecture suppresses uptake of PLEs into cancer cells. It has been shown that the uptake of PLEs is reduced by 60% when lipid rafts are blocked from forming. These features combined with lipid rafts providing rapid internalization of phospholipid drug conjugates, makes them an ideal target.

The compounds as disclosed herein, such as PLE analogs, may be LCFA mimetics. The molecules as disclosed herein have undergone extensive structure activity relationship (SAR) analysis related to targeting lipid rafts on tumor cells and have been shown to specifically bind to these regions. The molecules as disclosed herein provide entry directly into the cytoplasm and transit to the endoplasmic reticulum and mitochondria along the Golgi-apparatus-network within the cell cytoplasm. In some embodiments, the phospholipid drug conjugates (PDCs) as disclosed herein include a uniquely designed phospholipid ether conjugated to a novel combretastatin A (CBA) analogue via a cleavable linker. CBAs are potent cytotoxins that inhibit tubulin polymerization within the tumor cell as well as a demonstrated ability to disrupt the local vasculature around/within a tumor. In some embodiments, the compounds disclosed herein include a uniquely designed phospholipid ether conjugated to a flavagline (FLV) analogue via a cleavable linker. FLVs are potent cytotoxins that inhibit translation, cell cycle progression and induce apoptosis.

The compounds as detailed herein, or a pharmaceutically acceptable salt thereof, or composition comprising a compound as detailed herein may be used to treat cancer. In one aspect, the present disclosure provides a method of treating cancer in a subject in need thereof, comprising administering an effective amount of a compound as detailed herein, or a pharmaceutically acceptable salt thereof, or composition comprising a compound as detailed herein.

In another aspect, the present disclosure provides compounds, or pharmaceutically acceptable salts thereof, as disclosed herein for use in treating cancer in a subject in need thereof.

In another aspect, the present disclosure provides use of compounds, or pharmaceutically acceptable salts thereof, as disclosed herein for manufacturing a medicament for treating cancer in a subject in need thereof.

The cancers that may be treated with the compounds as detailed herein, or a pharmaceutically acceptable salt thereof, or composition comprising a compound as detailed herein include, but are not limited to: breast cancer including male breast cancer; digestive/gastrointestinal cancers including anal cancer, appendix cancer, extrahepatic bile duct cancer, gastrointestinal carcinoid tumor, colon cancer, esophageal cancer, gallbladder cancer, gastric cancer, gastrointestinal stromal tumors (“gist”), Islet cell tumors, adult primary liver cancer, childhood liver cancer, pancreatic cancer, rectal cancer, small intestine cancer, and stomach (gastric) cancer; endocrine and neuroendocrine cancers including pancreatic adenocarcinoma, adrenocortical carcinoma, pancreatic neuroendocrine tumors, Merkel cell carcinoma, non-small cell lung neuroendocrine tumor, small cell lung neuroendocrine tumor, parathyroid cancer, pheochromocytoma, pituitary tumor and thyroid cancer; eye cancers including intraocular melanoma and retinoblastoma; genitourinary cancer including bladder cancer, kidney (renal cell) cancer, penile cancer, prostate cancer, transitional cell renal pelvis and ureter cancer, testicular cancer, urethral cancer and Wilms tumor; germ cell cancers including childhood central nervous system cancer, childhood extracranial germ cell tumor, extragonadal germ cell tumor, ovarian germ cell tumor and testicular cancer; gynecologic cancers including cervical cancer, endometrial cancer, gestational trophoblastic tumor, ovarian epithelial cancer, ovarian germ cell tumor, uterine sarcoma, vaginal cancer and vulvar cancer; head and neck cancers including hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, parathyroid cancer, pharyngeal cancer, salivary gland cancer and throat cancer; leukemias including adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; lymphomas including AIDS-related lymphoma, cutaneous t-cell lymphoma, adult Hodgkin lymphoma, childhood Hodgkin lymphoma, Hodgkin lymphoma during pregnancy, mycosis fungoides, adult non-Hodgkin lymphoma, childhood non-Hodgkin lymphoma, non-Hodgkin lymphoma during pregnancy, primary central nervous system lymphoma, Sézary syndrome and Waldenström macroglobulinemia; musculoskeletal cancers including Ewing sarcoma, osteosarcoma and malignant fibrous histocytoma of bone, childhood rhabdomyosarcoma and soft-tissue sarcoma; neurological cancers including adult brain tumor, childhood brain tumor, astrocytomas, brain stem glioma, central nervous system atypical teratoid/rhabdoid tumor, central nervous system embryonal tumors, craniopharyngioma, ependymoma, neuroblastoma, primary central nervous system (CNS) lymphoma; respiratory/thoracic cancers including non-small cell lung cancer, small cell lung cancer, malignant mesothelioma, thymoma and thymic carcinoma; and skin cancers including Kaposi sarcoma, melanoma and squamous cell carcinoma. In particular embodiments, the cancer may be melanoma, lung cancer, colorectal cancer, breast cancer, or a combination thereof.

In another embodiment, the cancer may comprise one or more CTCs. The one or more CTCs may be selected from the group consisting of a breast cancer, a lung cancer, a thyroid cancer, a cervical cancer, a melanoma, a squamous cell carcinoma, a prostate cancer, a pancreas cancer, a colorectal cancer, and a cancer stem cell, and a malignant plasma cell.

In another embodiment, the cancer may be metastatic. In particular embodiments, the metastatic cancer may be selected from the group consisting of a breast cancer, a lung cancer, a melanoma, and a colorectal cancer.

In another embodiment, the cancer may be a cancer stem cell. In particular embodiments, the cancer stem cell may be derived from the group consisting of a breast cancer, a lung cancer, a melanoma, and a colorectal cancer.

In some embodiments, the lung cancer may comprise small cell lung cancer, non-small cell lung cancer, or a combination thereof.

In some embodiments, the melanoma may comprise superficial spreading melanoma, nodular melanoma, lentigo maligna melanoma, acral lentiginous melanoma, amelanotic melanoma, nevoid melanoma, spitzoid melanoma, desmoplastic melanoma, or a combination thereof.

In some embodiments, the colorectal cancer may comprise adenocarcinoma.

In some embodiments, a compound of formula (I-a), (l-a-1), (l-a-2), or (l-a-3) as detailed herein, or a pharmaceutically acceptable salt thereof, or composition comprising the compound as detailed herein may be used to treat melanoma, lung cancer, colorectal cancer, or a combination thereof.

In some embodiments, the breast cancer may comprise invasive breast ductal carcinoma, metastatic breast cancer, inflammatory breast cancer, triple negative breast cancer, ductal carcinoma in situ, or a combination thereof. In further embodiments, the cancer is breast cancer, the subject may be estrogen receptor positive, both estrogen receptor negative and progesterone receptor negative, expresses HER2 (HER2+), does not express HER2 (HER2-), or a combination thereof. In some embodiments, a compound of formula (l-b),(l-b-1),(l-b-2),or (l-b-3) as detailed herein, or a pharmaceutically acceptable salt thereof, or composition comprising the compound as detailed herein may be used to treat breast cancer.

In some embodiments, a compound of formula (l-c),(l-c-1), (l-c-2), or (l-c-3) as detailed herein, or a pharmaceutically acceptable salt thereof, or composition comprising the compound as detailed herein may be used to treat melanoma, lung cancer, colorectal cancer, breast cancer, or a combination thereof.

In some embodiments, the subject is a human, such as an adult and an infant. In some embodiments, the subject is an animal, such as a mammal.

The methods may include administering a compound as detailed herein, or a pharmaceutically acceptable salt thereof, or composition comprising a compound as detailed herein in amounts as detailed herein. In some embodiments, the methods include administering about 0.0001 to about 1000 mg/kg of a compound as detailed herein, or a pharmaceutically acceptable salt thereof.

Useful dosages of the compound(s) in the composition can be determined by comparing their in vitro activity and in vivo activity in animal models thereof. Methods for the extrapolation of effective dosages in rodents, pigs, and other animals, to humans are known to the art; for example, see U.S. Pat. No. 4,938,949.

Actual dosage levels of the compounds in the therapeutic compositions of as detailed herein can be varied so as to obtain an amount of the compound(s) which is effective to achieve the desired therapeutic response for a particular patient, compositions and mode of administration. The selected dosage level and the amount of the present compounds, or a pharmaceutically acceptable salts thereof, for use in treatment may vary with the particular compound or salt selected, the route of administration, the disease or condition being treated, the age and condition of the subject being treated, the severity of the condition being treated, and the condition and prior medical history of the patient being treated. In cases of administration of a pharmaceutically acceptable salt, dosages may be calculated as the free base. However, it is within the skill of the art to start doses of the compound at levels lower than required to achieve the desired therapeutic effect and to gradually increase the dosage until the desired effect is achieved. In certain situations, the disclosed compounds may be administered in amounts that exceed the dosage ranges described herein in order to effectively and aggressively treat particularly aggressive diseases or conditions.

In some embodiments, the compounds, or pharmaceutically acceptable salts thereof, or pharmaceutical compositions as disclosed herein may be administered by oral administration or intravenous administration. In general, however, a suitable dose will often be in the range of from about 0.0001 mg/kg to about 1000 mg/kg, such as from about 0.001 mg/kg to about 10.0 mg/kg. For example, a suitable dose may be in the range from about 0.001 mg/kg to about 5.0 mg/kg of body weight per day, such as about 0.01 mg/kg to about 1.0 mg/kg of body weight of the recipient per day, about 0.01 mg/kg to about 3.0 mg/kg of body weight of the recipient per day, about 0.1 mg/kg to about 5.0 mg/kg of body weight of the recipient per day, about 0.2 mg/kg to 4.0 mg/kg of body weight of the recipient per day. The compound may be administered in unit dosage form; for example, containing 1 to 100 mg, 10 to 100 mg, or 5 to 50 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Suitable in vivo dosages to be administered and the particular mode of administration may vary depending upon the age, weight, the severity of the affliction, and mammalian species treated, the particular compounds employed, and the specific use for which these compounds are employed. The determination of effective dosage levels to achieve the desired result may be accomplished by known methods, for example, human clinical trials, in vivo studies and in vitro studies. For example, the effective dosages of compounds disclosed herein, or pharmaceutically acceptable salts thereof, may be determined by comparing their in vitro activity, and in vivo activity in animal models. Such comparison may be done by comparison against an established drug.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, FIPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

Compounds, salts, and compositions disclosed herein may be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

The compound(s) as detailed herein, or a pharmaceutically acceptable salt thereof, or composition comprising the compound(s) as detailed herein can be administered to humans and other mammals by a variety of known routes, including without limitation orally, rectally, parenterally, intracisternally, intravaginally, transdermally (e.g. using a patch), transmucosally, sublingually, pulmonary, intraperitoneally, topically (as by powders, ointments or drops), bucally or as an oral or nasal spray. The terms “parenteral” or “parenterally,” as used herein, refers to modes of administration which include intravenous, intramuscular, intraperitoneal, intrasternal, subcutaneous and intraarticular injection and infusion.

The compositions described herein may be administered with additional compositions to prolong stability, delivery, and/or activity of the compositions, or combined with additional therapeutic agents, or provided before or after the administration of additional therapeutic agents. Combination therapy includes administration of a single pharmaceutical dosage formulation containing one or more of the compounds described herein and one or more additional pharmaceutical agents, as well as administration of the compounds and each additional pharmaceutical agent, in its own separate pharmaceutical dosage formulation. For example, the compounds as detailed herein may be administered to a subject with an additional anti-cancer drug as detailed herein.

Compounds as detailed herein, or pharmaceutically acceptable salts thereof, can also be administered in the form of liposomes. As is known in the art, liposomes are generally derived from phospholipids or other lipid substances. Liposomes are formed by mono- or multilamellar hydrated liquid crystals which are dispersed in an aqueous medium. Any, physiologically acceptable and metabolizable lipid capable of forming liposomes can be used. The present compositions in liposome form can contain, in addition to a compound described herein, anti-cancer drugs, stabilizers, preservatives, excipients and the like. The preferred lipids are natural and synthetic phospholipids and phosphatidyl cholines (lecithins) used separately or together. Methods to form liposomes are known in the art. See, for example, Prescott, Ed., Methods in Cell Biology, Volume XIV, Academic Press, New York, N.Y. (1976), p. 33 et seq. Such compositions will influence the physical state, solubility, stability, rate of in vivo release, and rate of in vivo clearance.

In one method of the present disclosure, a pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in proximity to the therapeutic target, for example liver, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

### 5. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1. Materials and Methods

In vitro uptake of CLR2000045 was assessed using MCF-7 breast cancer cells and normal human dermal fibroblasts (NHDF) cells and was measured via LC/MS/MS. The breast cancer cells were maintained in minimum essential medium supplemented with 10% FBS. All cells were maintained at 37° C. and 5% CO₂. Cells were incubated with 1 µM of drug and reported values were the average of triplicate assessments. In vitro cytotoxicity was determined by Cell Titer-Glo® assay using MCF-7 breast cancer cells and Hs578T triple negative breast cancer cells.

In vitro uptake and release of CLR180099 were assessed using A549 tumor cells, HCT116 tumor cells, and normal human dermal fibroblasts (NHDF) cells and measured via LC/MS/MS. Cells were incubated with 1 µM of drug and reported values were the average of triplicate assessments. In vitro cytotoxicity was determined by Cell Titer-Glo® assay.

In an efficacy screening model using chicken embryos in vivo, 72 µM of CLR2000045 was administered to determine efficacy against MCF-7 tumors and compared against vehicle control and paclitaxel positive control at 50 µM. CLR2000045 was applied topically to the embryo casing. Fertilized White Leghorn eggs were incubated at 37.5° C. with 50% relative humidity for 9 days. At that moment (E9), the chorioallantoic membrane (CAM) was dropped down by drilling a small hole through the eggshell into the air sac, and a 1 cm² window was cut in the eggshell above the CAM. At least 20 eggs (depending on embryo surviving rate after 9 days of development, there could be more than 20 eggs per group) were used for each group. Because some embryo deaths may occur after tumor grafting or may be related to a defective tumor graft, data may be collected with less than 20 eggs per group (minimum of 15 eggs per group). Tumor cells were cultivated in DMEM supplemented with 10% FBS and 1% penicillin/streptomycin. On day E9, the cells were detached with trypsin, washed with complete medium and suspended in graft medium. An inoculum of 3x10⁶ cells were added onto the CAM of each egg (E10) per group as appropriate and then eggs were randomized into groups.

Embryonic viability was checked daily. The number of dead embryos were also counted on E18, in combination with the observation of eventual visible gross abnormalities, to evaluate treatment-induced embryo toxicity. The final death ratio and a Kaplan-Meyer curve were calculated for all groups. Any visible abnormality observed was also noted. On day E18, the upper portion of the CAM (with tumor) was removed from all viable embryos with tumors, washed with PBS buffer and then directly transferred in PFA (fixation for 48 hr). After that, tumors were carefully cut away from normal CAM tissue and weighed.

In vivo efficacy was further assessed in R2G2 mice bearing HCC70 triple negative breast cancer (TNBC) xenografts. Three doses (1 mg/kg) given once, twice or 3 times per week for 2 weeks of CLR2000045 were assessed. CLR2000045 was administered systemically by injection of the tail vein. Each group contained 10 mice. Tumor volume was monitored for efficacy and body weight for tolerability. Survival was also monitored.

CLR180099 was administered intravenously (IV) to healthy C57BL/6 mice to determine the maximum tolerated dose (MTD) as compared to the FLV molecule alone. The vehicle used to administer CLR180099 was PBS in this case, however any pharmaceutically suitable vehicle may be used. Each group contained 5 mice. In vivo efficacy was assessed in athymic nude mice bearing HCT 116 xenografts. The mice were flank models which were developed by injecting the hind flank of the mice with about 1x10⁶ of the cells resuspended in 5 mL of 1.2% methylcellulose. The study was initiated when group mean tumor volume reached about 120 mm³. Tumor volume was measured using calipers - measurements of the length, width, and depth of the tumor were used to calculate the tumor volume. Two doses (2 mg/kg given 2 times or 2 mg/kg given 3 times) of CLR180099 were assessed. Each group contained 10 mice. Tumor volume was monitored for efficacy and body weight for tolerability. Total conjugated CLR180099 and free FLV were determined via mass spectrometry.

### Example 2. Phospholipid Lipid Ether Delivery Vehicle Shows Specificity for a Broad Range of Tumor Cells

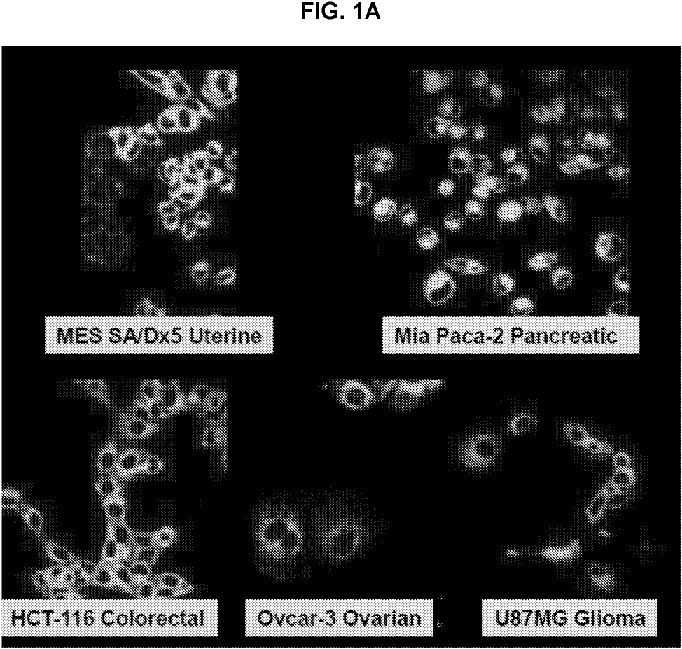

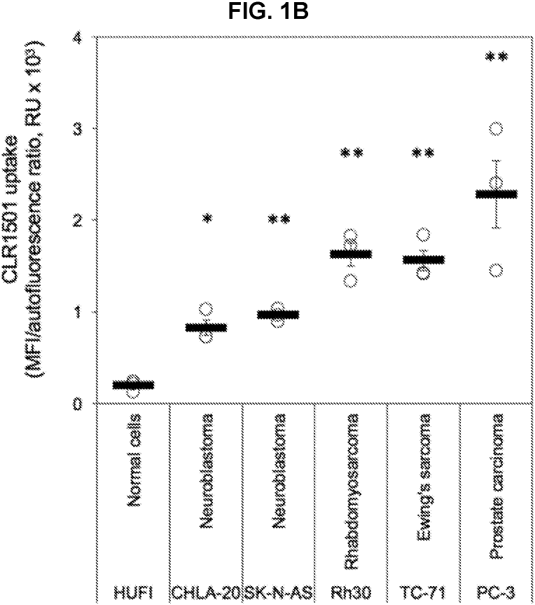

To demonstrate the uptake of PDCs in various tumor cell lines, various tumor cell lines, such as MCT-116, MeS SA/Dx5, Mla PaCa-2, Ovcar-3 and U-87MG, were incubated with 5 µM of CLR1501 (PLE plus a BODIPY fluorescent payload) for 24 hours at 37° C. in complete media. Each cell line can have a slightly different media to optimize growth, any suitable media known in the art can be used for each cell line. All cells were maintained at 37° C. in an appropriate medium supplemented with 10% FBS and 5% CO₂. CLR1501 was excited and then detected with an Alexa-Fluor 488 filter. CLR1501 was highly localized in all the different tumor cell lines (FIG. 1A and FIG. 1B). This has been repeated in over 100 tumor cell lines, such as MM.IS, MM.IR, RPM18226, U266, and NCIH929, Panc-1, A375, PC-3, Caki-2, HCT-116, A549, metastatic PC-3, MDA-MB-231, HT-29, SV-40, CNS-1, BxPC3, MCF-7, LuCap, LNCap, MES SA/Dx5, Capan1, HTB-77, Lan5, CHLA-20, NB1691, and SK-N-AS, with similar results. CLR1501 was administered to different cancer cell lines and a normal human skin fibroblast line in vitro. Twenty-four hours later, CLR1501 exhibited from five to nine-fold preferential uptake in these cancer cell lines in vitro compared to normal fibroblasts. Retained CLR1501 was associated with plasma and organelle membranes.

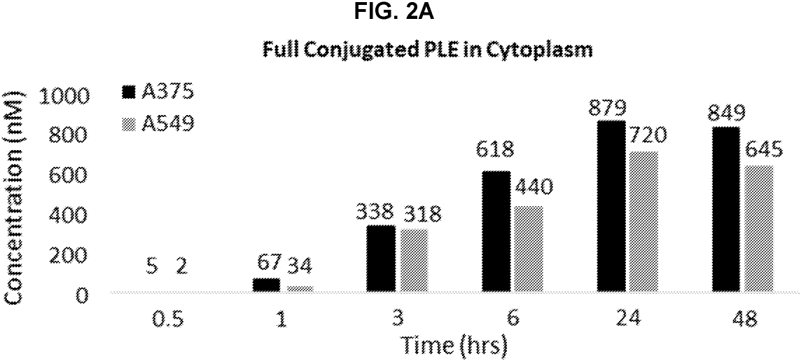

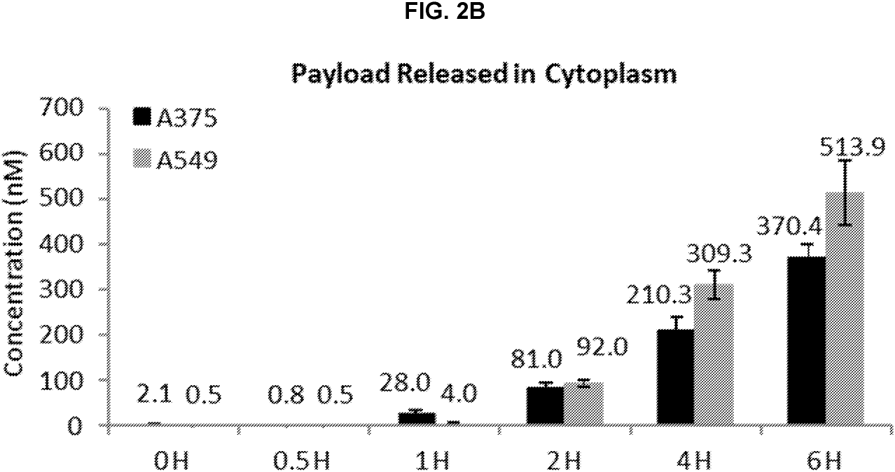

In vitro uptake and release with a cytotoxic payload was measured in A375 and A549 cell lines by incubating them with 2 µM of a cytotoxic small molecule PDC with semi-stable linker (CLR2208, “PDC-SM1”) for 48 hours at 37° C. in complete media. Uptake of PDC-SM1 was measured by LC/MS/MS. PDC-SM1 demonstrated uptake initiating within 30 minutes. 20-40% of conjugate exposed to cells was measured in the tumor cell cytoplasm within 24 hrs (FIG. 2A). CLR2206 (“PDC-SM2,” same as PDC-SM1 except with a cleavable linker) was then utilized to measure release of payload within tumor cells. CLR2200 (“PDC-SM3”) was also studied. Measurable release of the small molecule payload occurred between 1 to 2 hours post incubation (FIG. 2B). Negligible release of payload occurred in media (<1 nM). These results indicated that phospholipid ether molecules have the ability to target a wide range of tumors and PDCs have the ability to achieve an uptake of 20-40% of the exposed drug into tumor cell lines.

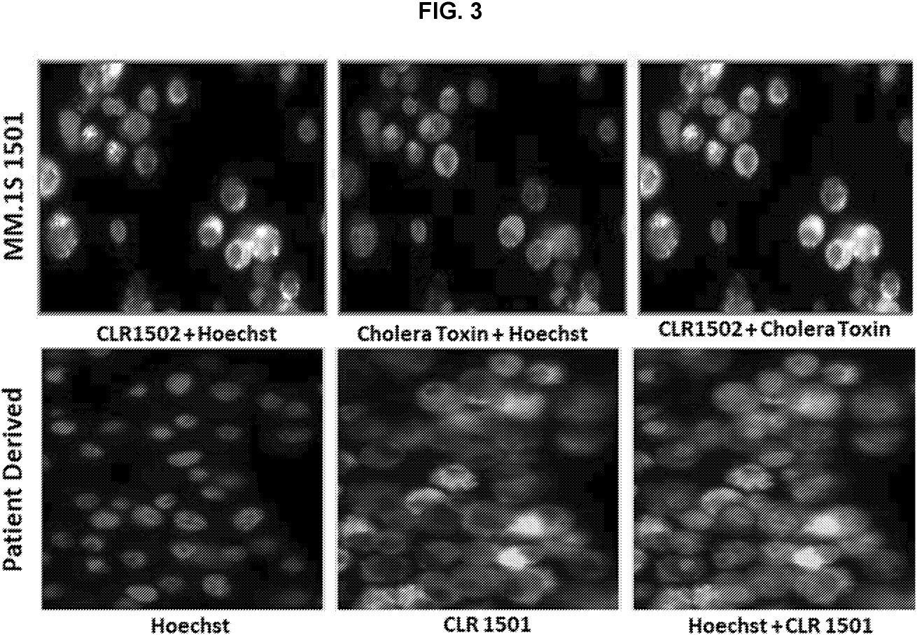

To measure uptake via lipid rafts on tumor cells multiple myeloma cells were incubated with CLR1502 (near infrared molecule bound to the PLE) for 24 hours at 37° C. The next day, the cells were washed and co-stained with nucleus stain (Hoescht 33342). Using cholera toxin subunit B, they were further stained for the presence of lipid rafts. The cells were incubated with cholera toxin subunit B for 24 hours. Additionally, to measure uptake via lipid rafts on primary tumor samples, patient derived multiple myeloma cells were stained with Hoescht 33342 and incubated with CLR1501 (FIG. 3). These results demonstrate that PDC uptake was linked to lipid rafts on tumor cell membranes in both cell lines and primary tumor samples.

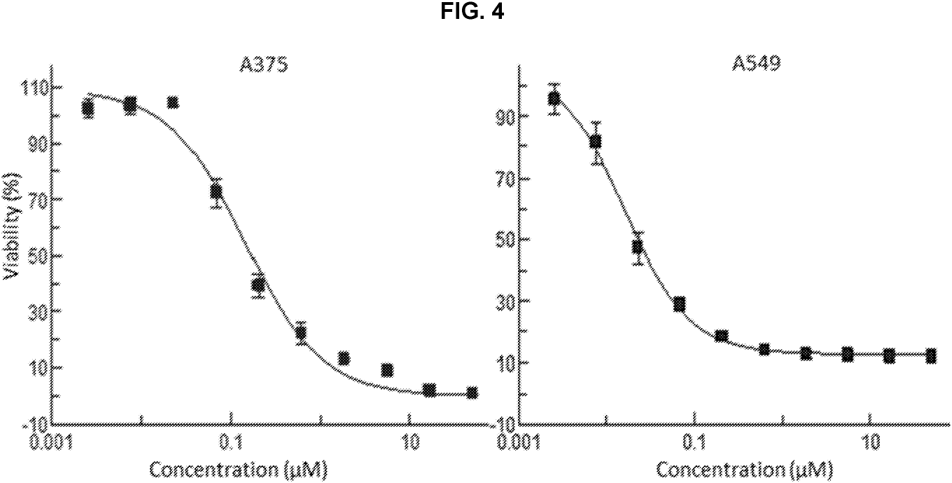

In vitro efficacy with cytotoxic payloads was measured. PDC-SM2 demonstrated sub-micromolar activity (concentration measured based on full conjugate concentration incubated on cells) against melanoma (A375) and lung cancer (A549) cells. PDC-SM2 showed less activity against melanoma than lung cancer (IC50s 0.131 vs 0.016) but was more potent (0% vs 12% viable cells remaining, FIG. 4). PDC-SM2 also showed similar activity and potency against colorectal cancer (HCT-116) cells as lung cancer with no activity against normal fibroblast cells. Therefore, PDCs show release of payload and strong nanomolar activity against tumor cells.

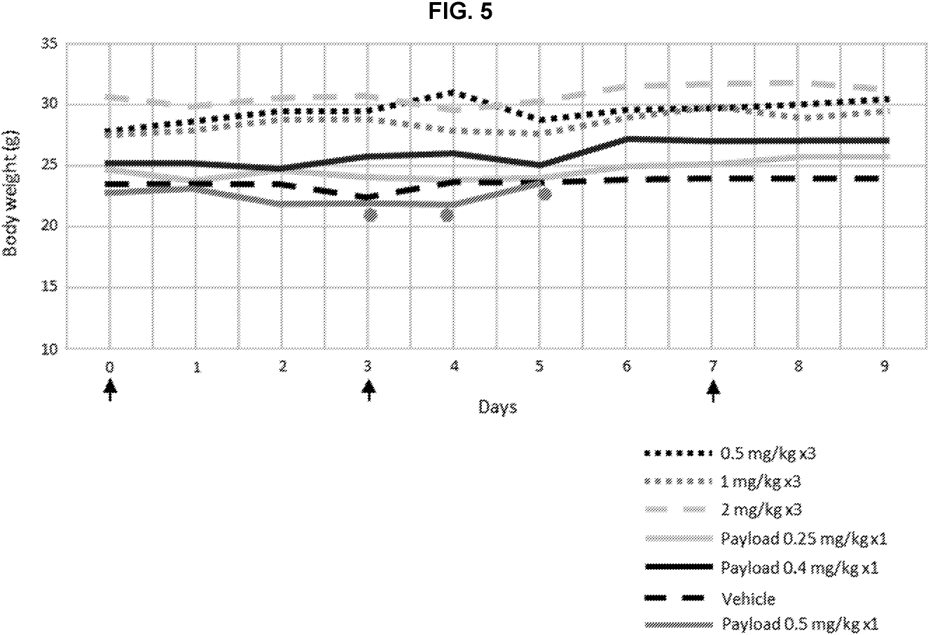

To determine whether cytotoxic PDCs are tolerated in vivo, C57BL/6 mice were dosed in the following manner: PDC-SM2 was dosed on days 0, 3 and 7 at dose levels of 0.5 mg/kg, 1.0 mg/kg, or 2.0 mg/kg; Payload alone was dosed on day 0 only at 0.25 mg/kg, 0.4 mg/kg, or 0.5 mg/kg; vehicle was dosed on day 0, 3 and 7. PDCs and vehicle control showed no toxicity or adverse events during repeat dosing as measured by changes in weight (no weight loss). Payload doses of 0.25 and 0.4 mg/kg were tolerated although there was some toxicity noted to the mice’s skin and coat. Payload dose of 0.5 mg/kg was not tolerated, two mice died by day 4 following a single infusion and all mice were sacrificed on day 5 (FIG. 5). These PDCs showed good plasma stability in human plasma. Plasma stability was measured using Cyprotex’s Plasma Stability assay. The samples were at a concentration of 1 µM and were incubated at 0, 15, 30, 60 and 120 minutes. A positive control compound which undergoes degradation in plasma was used. The percent of the compound remaining at each incubation time point was measured. PDC-SM2 showed some instability in mouse plasma which could result in some toxicity (TABLE 1). The present PDCs are well tolerated in vivo. Overall, PDCs offer a novel and unique approach to targeting small molecules to tumor cells.

Table 1

Plasma Stability Assessment

| Compound ID | Human T_{½} (min) | Mouse T_{½} (min) |
|---|---|---|
| PDC-SM2 | >400 | 199 |
| PDC-SM3 | >400 | >400 |
| Propantheline | 54 | 85 |

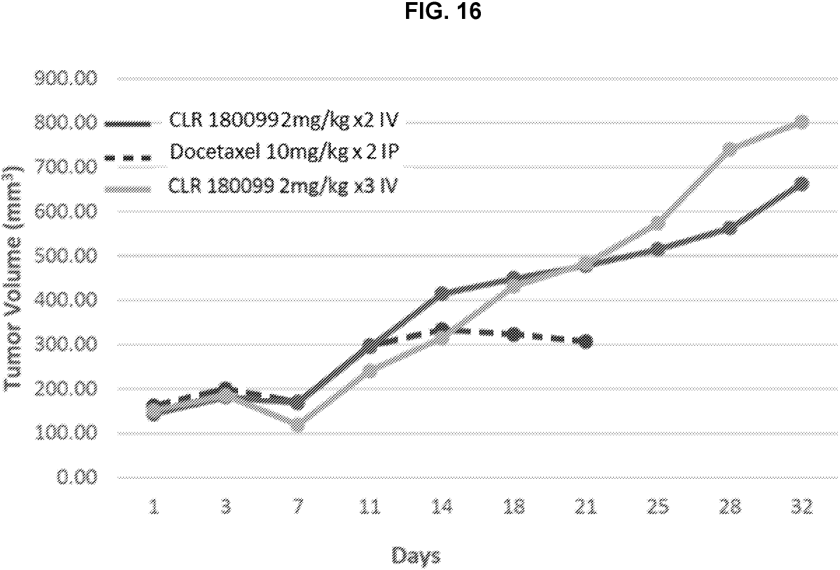

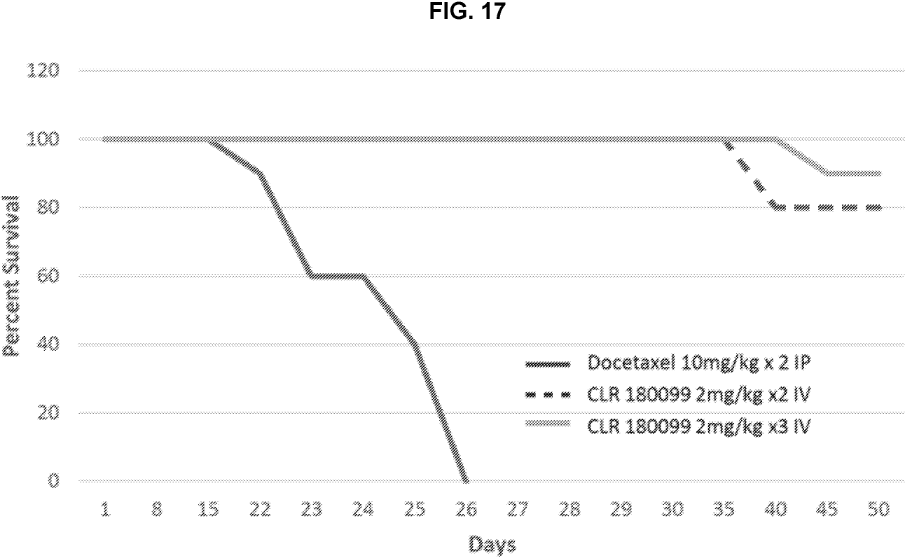

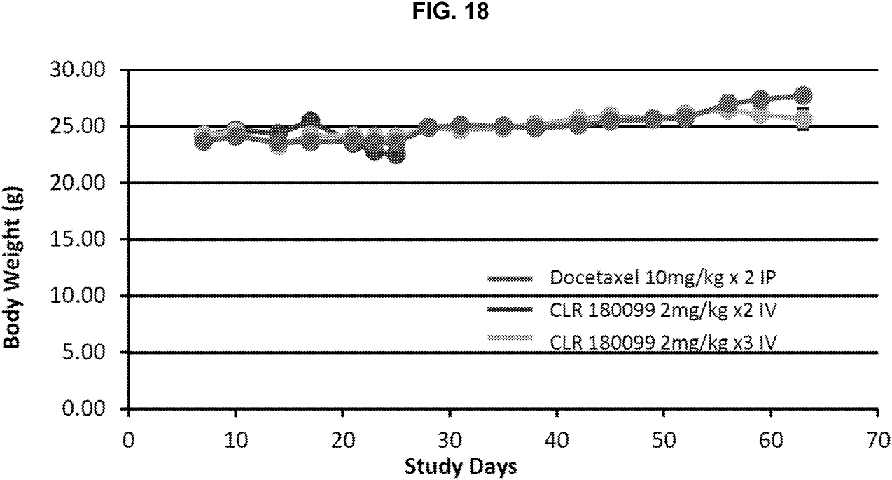

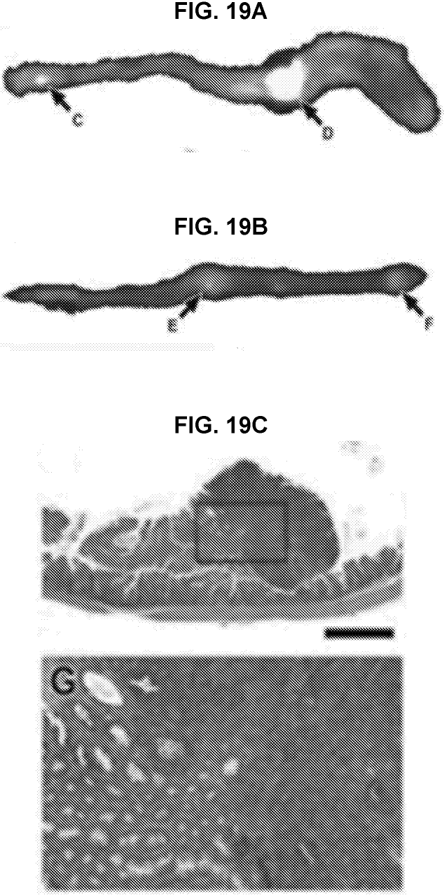

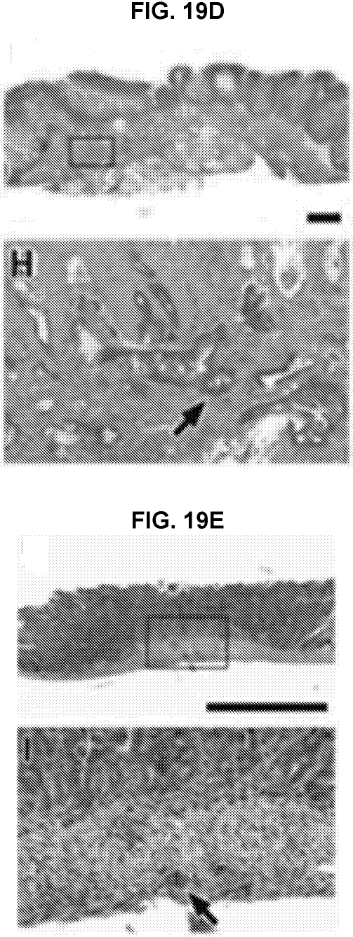

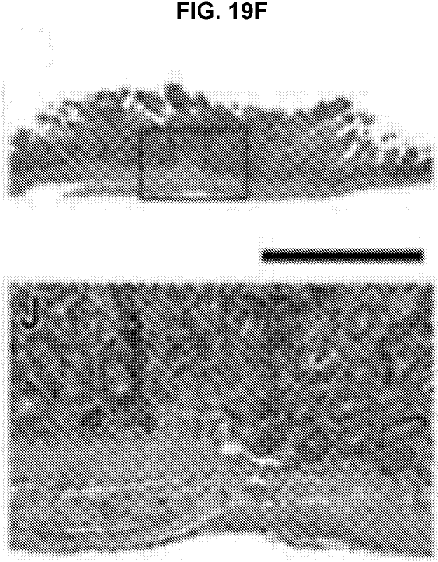

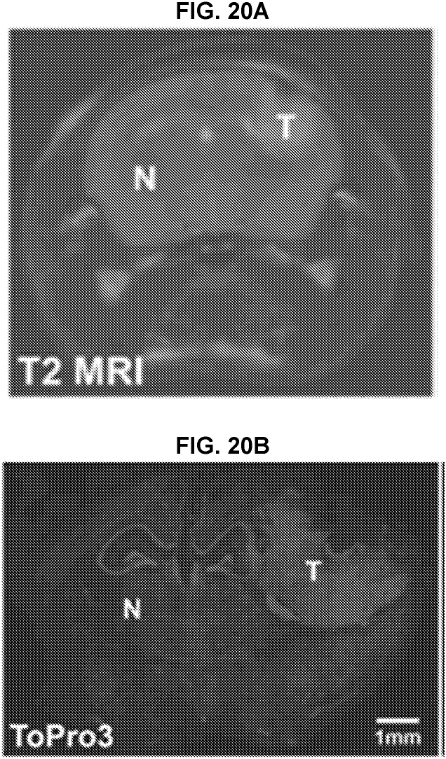

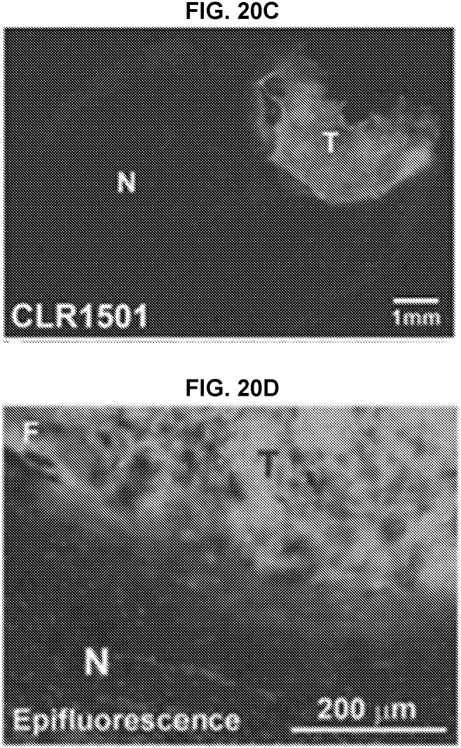

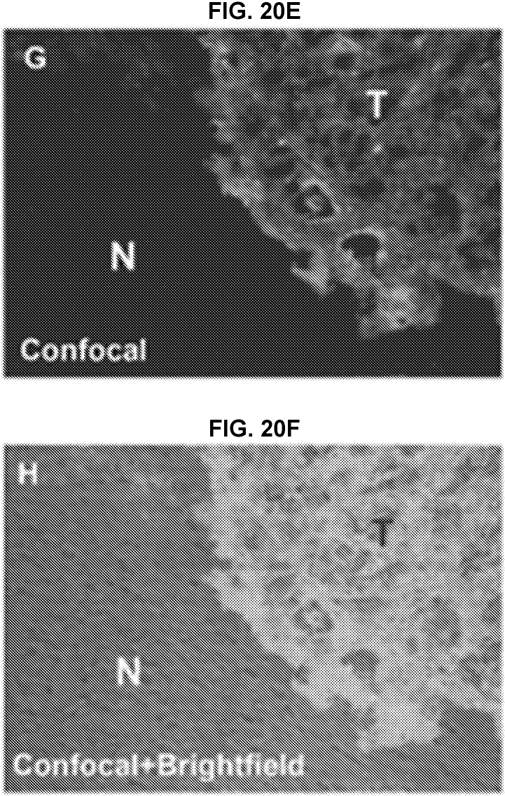

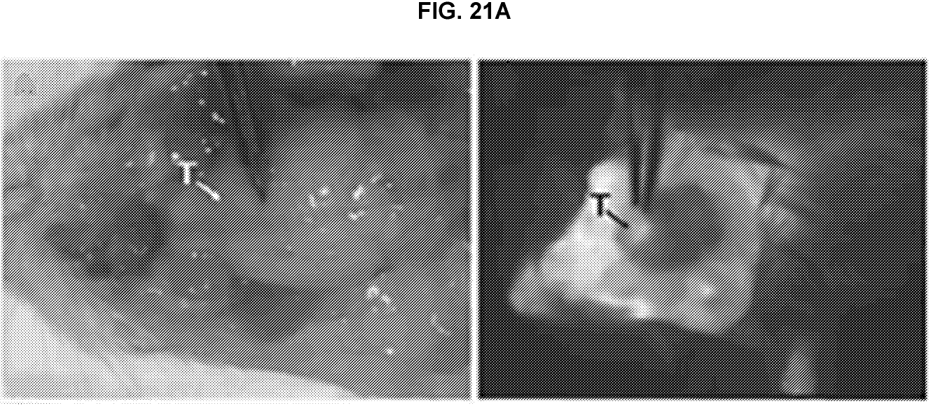

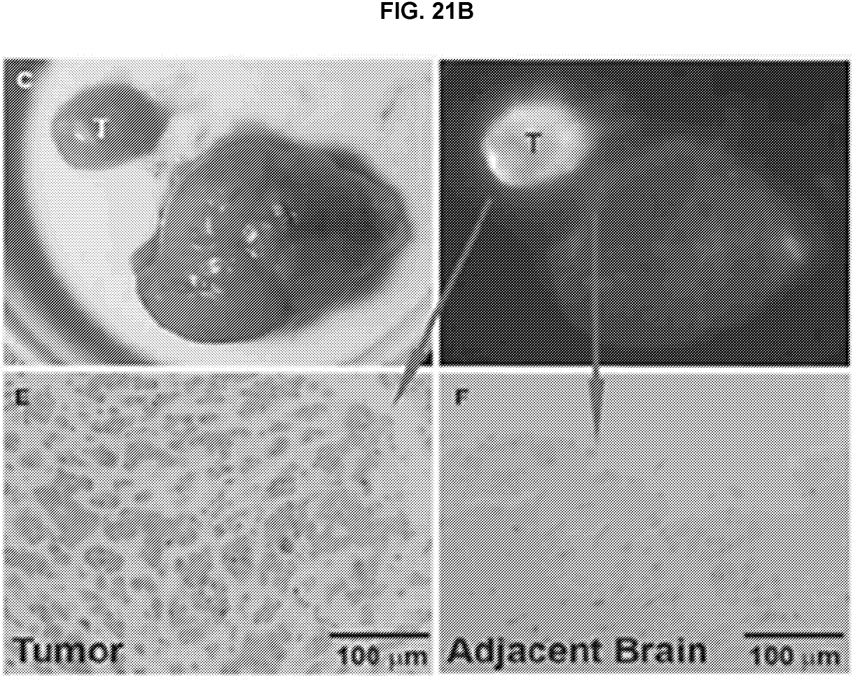

The selective uptake of CLR1502 was also measured in vivo in intestinal tumors. The entire colon and the distal segment of the small intestine was removed at necropsy 96 hours after administration of 50 µg of CLR1502 (FIG. 19A and FIG. 19B). CLR1502 was administered via tail vein injection. Areas of increased signal intensity were observed using the IVIS Spectrum, which allows for direct visualization of CLR1502 through the animal’s skin. Then after euthanizing the animal, the IVIS system-identified tissues were excised via microdissection and histology was performed to see tumor versus nontumor tissue and where the near infrared labeling occurred. These areas showed non-invasive (colon FIG. 19C; distal small intestine FIG. 19F) and invasive (colon FIG. 19D; distal small intestine FIG. 19E) tumors.

In other studies, CLR1502 accumulates in metastases and in regional lymph nodes. Following removal of the intestine, mesenteric fat, pancreas, and spleen were isolated en bloc. In one case, two metastatic tumor deposits of ~4 mm in size were noted within the mesentery. These lesions were easily visualized with the Fluobeam near-infrared imager. These lesions were confirmed to be metastatic malignant lesions on H&E. Regional lymphadenopathy was also shown to accumulated CLR1502 using the Fluobeam. No malignant cells were observed within these hyperplastic lymph nodes.

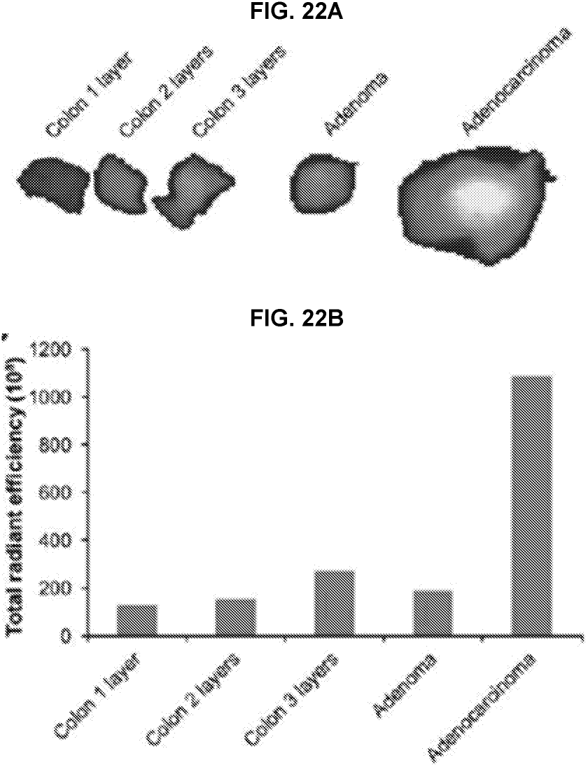

Tumor thickness does not account for the increased signal intensity noted in the intestinal cancers (FIG. 22A and FIG. 22B). Necropsy was performed 96 hours post injection of mice with 50 µg of CLR1502 per mouse. To examine the effect of tissue thickness, sections of normal appearing colon were layered upon each other. The radiant efficiency was measured to compare signal intensity between one, two, and three layers of normal colon and intestinal tumors. Note that one layer of normal colon is approximately 1 mm thick. Tissue thickness might account for the increased intensity seen in the adenomas, but does not account for the differences seen with the adenocarcinomas.

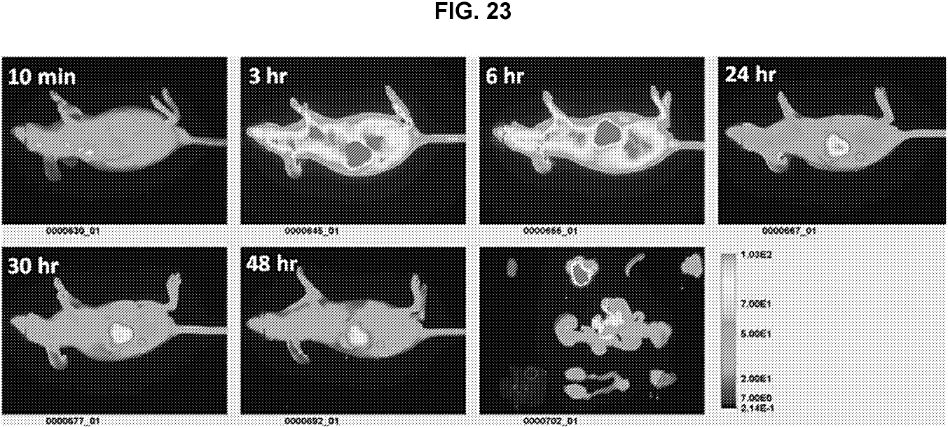

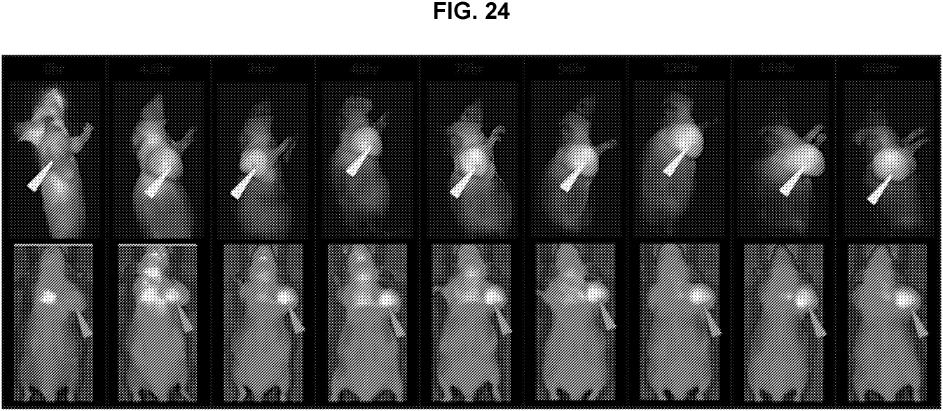

In vivo optical scanning of CLR1502 uptake in a colorectal carcinoma model demonstrated preferential retention in malignant compared to normal tissues. An athymic nude mouse bearing a colorectal carcinoma (HCT-116) xenograft was injected intravenously with 1 mg of CLR1502, and imaged using the Li-COR Pearl® Impulse system (FIG. 23). Fluorescence intensity (indicated by color bar) and biodistribution were determined in vivo over time.

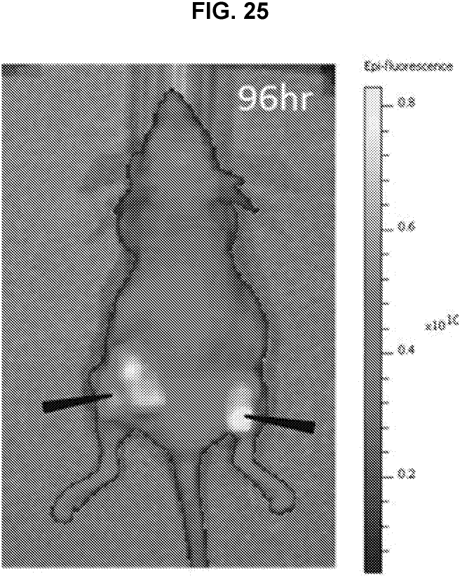

In vivo optical scanning of CLR1502 uptake in a breast cancer model demonstrated preferential retention in malignant compared to normal tissues. An athymic nude mouse bearing an orthotopic breast cancer xenograft (MDA-MB-231) was injected intravenously with approximately 80 µg of CLR1502 and imaged daily in vivo for seven days (168 hr) using Fluoptics Fluobeam® and IVIS® Spectrum systems (FIG. 24). The study results showed selective uptake and prolonged retention within the tumor (yellow and green arrows for Fluobeam and IVIS Spectrum, respectively) and the relative increased clearance from the normal tissue over time.

An athymic nude mouse bearing a lung cancer xenograft (H226 lung) on each flank was injected intravenously with approximately 50 µg of CLR1502 and imaged in epi-fluorescence mode with the IVIS Spectrum (FIG. 25). Note that at 96 hours the difference in radiant efficiency between the malignant and normal tissue creates sufficient contrast for tumor margin illumination, as indicated by black arrows.

### Example 3. CLR2000045 With a Combretastatin A-4 Analogue Improves Breast Cancer Therapy

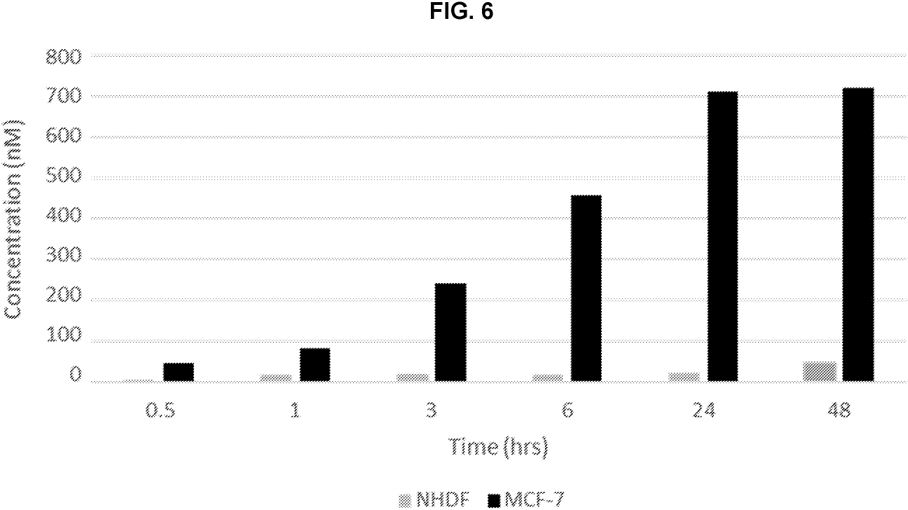

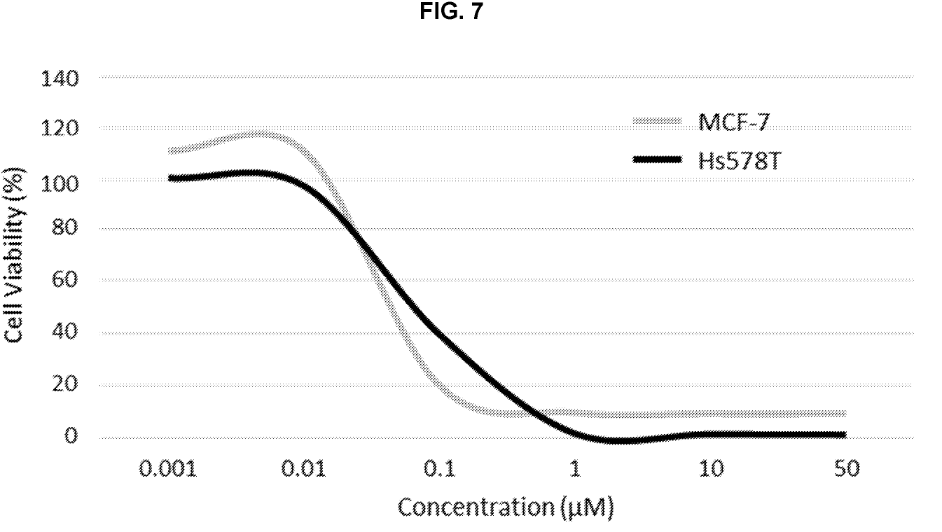

CLR2000045 shows significant uptake in tumor cells with minimal uptake in normal tissue. Release of the warhead showed approximately 50% release at each timepoint. Between 24 and 48 hours a steady state between uptake and release of the warhead was achieved (FIG. 6). CLR2000045 shows excellent activity and potency against two breast cancer cell lines (MCF-7 and Hs578T) with IC50s 76 nM and 51 nM, respectively (FIG. 7). The molecule also demonstrated activity against several other solid tumors, including lung cancer, melanoma and colorectal cancer. Half maximal inhibitory concentration (IC50) was measured in the cell lines (TABLE 2). Plasma stability of CLR2000045 was also measured (TABLE 3).

Table 2

IC50 Assessment

| Compound ID | Cell Type | | | | |
|---|---|---|---|---|---|
| | A375 | A549 | HCT116 | MCF7 | NHDF |
| CLR2013 | 0.443 | 0.445 | 0.451 | 0.282 | >50 |
| CLR2000045 | 0.610 | 1.592 | 1.886 | 1.082 | >50 |
| CLR2010 | 0.385 | 0.457 | 0.449 | 0.356 | >50 |

Table 3

Plasma Stability Assessment

| Compound ID | Human | Mouse |
|---|---|---|
| | t_{½} (min) | t_{½} (min) |
| CLR2013 | >400 | 77 |
| CLR2000045 | >400 | >400 |
| Propantheline | 77 | 57 |

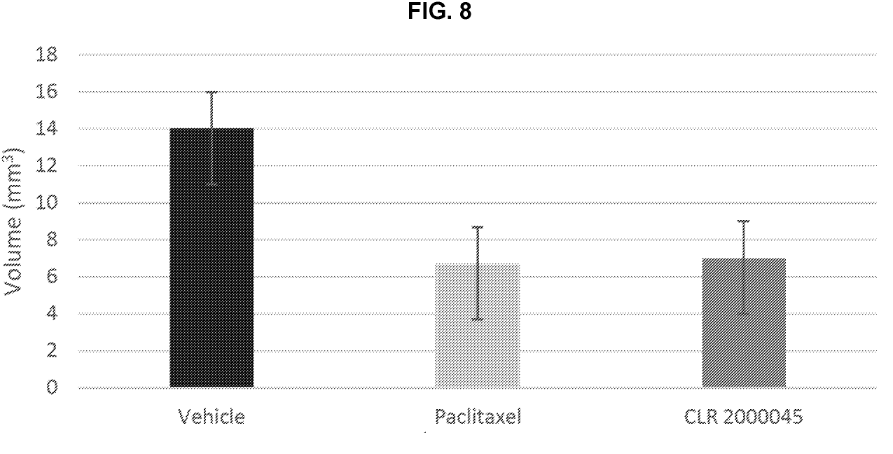

Fertilized White Leghorn chicken eggs (20/dose group) were incubated at 37.5° C. for 9 days. MCF-7 cells were cultured under standard conditions prior to implanting. An inoculum of 3x10⁶ MCF-7 cells were added to the chorioallantoic membrane on day 10. Eggs were then randomized to treatment groups and treated 4 times (day 11, 13, 15 and 17) under the following conditions: vehicle, paclitaxel 50 µM per dose, and CLR2000045 72 µM per dose. CLR2000045 showed similar activity to paclitaxel in this screening model (FIG. 8).

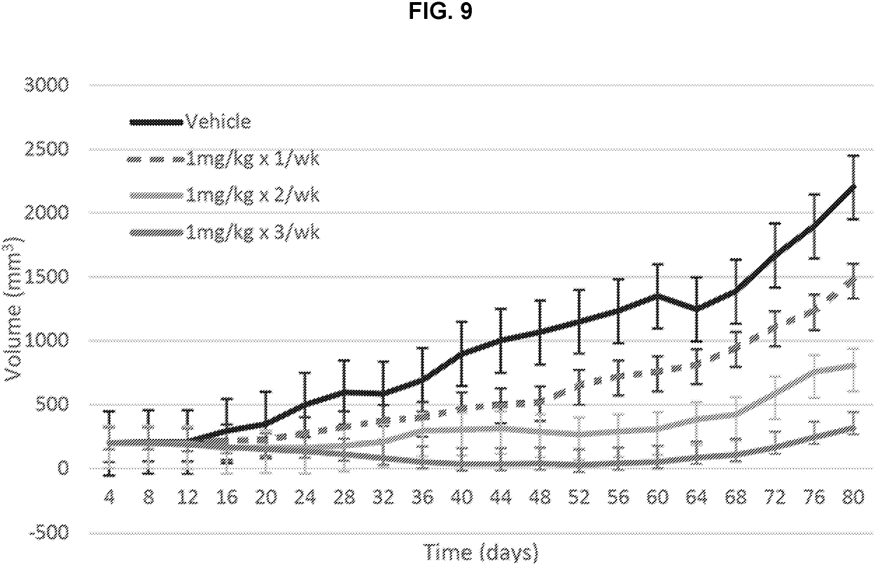

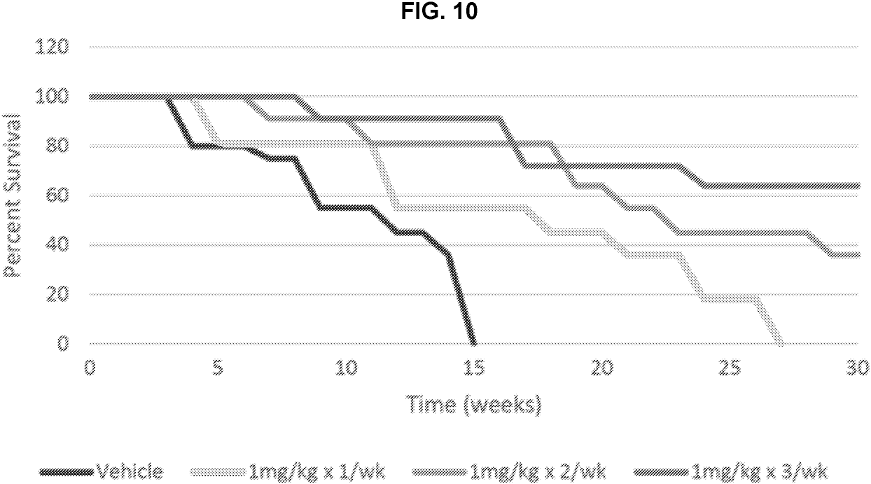

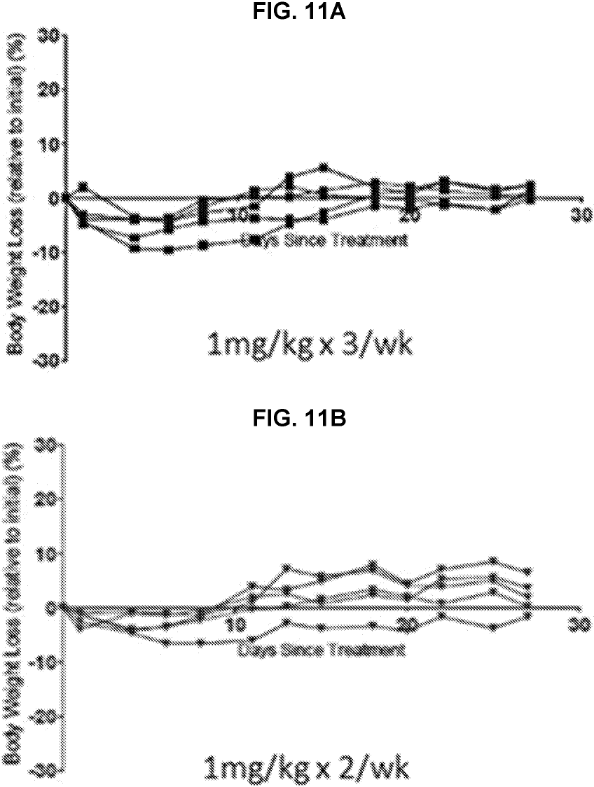

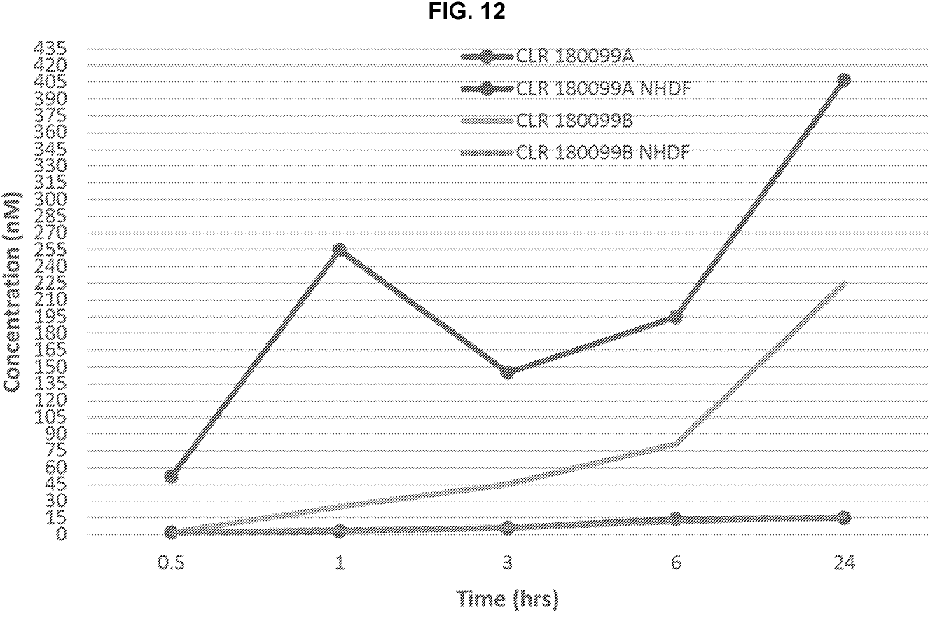

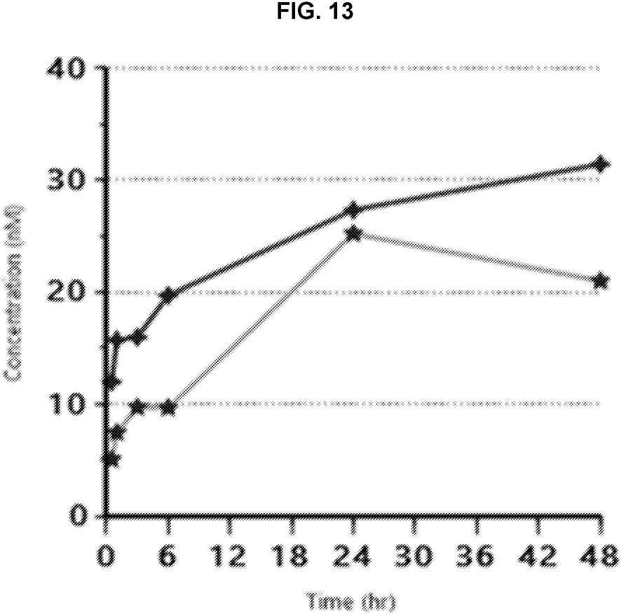

The study was initiated when group mean tumor volume reached ~200 mm³ (Day 4). CLR2000045 was dosed IV at the following doses: 1 mg/kg on either day 5 and 12 or day 5, 8, 12 and 15 or day 5, 7, 9, 12, 14, and 16. CLR2000045 demonstrated a dose response reduction in tumor volume from dose group 1 to dose group 3 (3 times per week for 2 weeks) and the highest dose tested showed near 100% eradication of the tumor. The 2 highest dose groups showed a statistically significant reduction in tumor volume as compared to the vehicle control (p≤0.05 and p≤0.01 respectively) (FIG. 9). The Kaplan-Meier curve shows that treatment with CLR2000045 at 1 mg/kg three times per week for 2 weeks resulted in a significant increase in survival as compared to vehicle and 1 time per week dosing (p≤0.001, p ≤0.05, respectively). 1 mg/kg twice a week for two weeks resulted in a significant increase as compared to vehicle (p≤0.05; FIG. 10). Changes in body weight post treatment were measured in the (HCC70) mouse xenograft model (FIG. 11A and FIG. 11B).

CLR2000045 demonstrates significant uptake and release of payload (20-40% of exposed drug) in tumor cell lines while minimal uptake occurs in normal cells. CLR2000045 shows potent in vitro activity against multiple breast cancer cell lines. CLR2000045 demonstrated potent in vivo activity against a triple negative breast cancer model (HCC70) and a metastatic adenocarcinoma breast cancer model (MCF-7). CLR2000045 provided a statistically significant survival benefit in the TNBC (HCC70) model and the two highest doses were shown to be well tolerated as measured by body weight loss. Together these data demonstrate the potent in vitro and in vivo activity of CLR2000045 against a variety of breast cancer cell lines and animal models and warrants the continued development of this PDC.

### Example 4. CLR180099 Improves the Safety and Efficacy of Antitumor Drugs Against Colorectal Tumors

CLR180099 showed excellent activity and potency against breast cancer and lung cancer with IC50s of 0.024 and 0.011, respectively (FIG. 12). The compound also demonstrated activity against several other solid tumors, including melanoma and colorectal cancer. Plasma stability of CLR1800095, CLR180099A, and CLR180099B was measured in mice and humans (TABLE 4). CLR1800095 showed some instability in mouse plasma which could result in some toxicity.

Table 4

Plasma Stability Assessment

| Compound ID | Human | Mouse |
|---|---|---|
| | t_{½} (min) | t_{½} (min) |
| CLR1800095 | >400 | 199 |
| CLR180099A | >400 | >400 |
| CLR180099B | >400 | >400 |
| Propantheline | 54 | 85 |

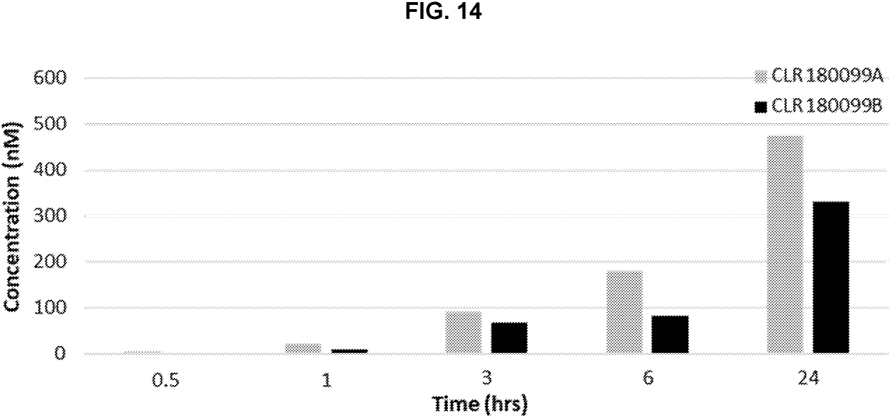

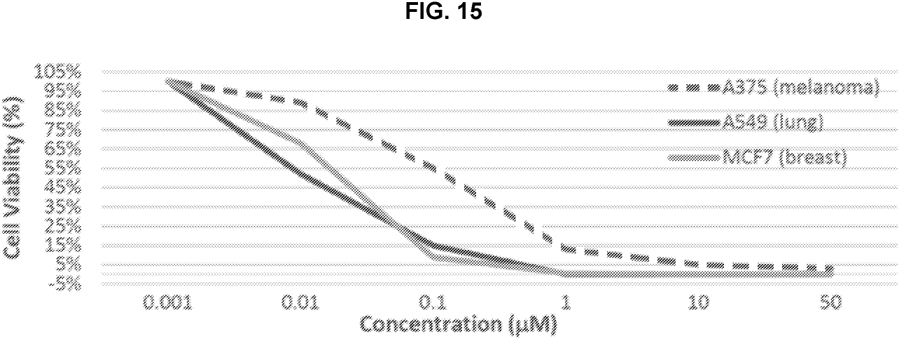

The study was initiated when group mean tumor volume reached -120 mm³ (Day 1). CLR180099 was dosed IV at 2 mg/kg on either day 1 and 4 or day 1, 3 and 5. Docetaxel was dosed at 10 mg/kg on day 1 and 4. CLR180099 demonstrated similar or better reduction in tumor volume than docetaxel and demonstrated a dose dependent effect. The docetaxel arm experienced multiple deaths starting at day 18 and ending at day 26 (FIG. 16). The Kaplan-Meier curve shows that treatment with CLR180099 at 2 mg/kg day 1 and 4 or day 1, 3 and 5 resulted in a significant increase in survival as compared to docetaxel (FIG. 17, log-rank test, p ≤0.001). As measured by body weight loss, all mice treated with CLR180099 (both doses) demonstrated normal body weight growth throughout the study (FIG. 18). Five mice per group were dosed at each dose level. Both PDCs were tolerated up to dose of 10 mg/kg with all mice alive and showing no end organ toxicities (TABLE 5). The payload alone was not tolerated at doses above 0.5 mg/kg (all mice died at 0.5 mg/kg).

Table 5

In vivo tolerability

| | | | | | |
|---|---|---|---|---|---|
| mg/kg | 0.1 | 0.5 | 1 | 5 | 10 |
| FLV | 5 | 0 | 0 | 0 | 0 |
| CLR180099A | 5 | 5 | 5 | 5 | 5 |
| CLR180099B | 5 | 5 | 5 | 5 | 5 |

CLR180099 demonstrated significant uptake and release of payload (20-40% of exposed drug) in tumor cell lines while minimal uptake occurred in normal cells. CLR180099 showed potent in vitro activity against various solid tumors, including lung cancer (A549), breast cancer (MCF7), and melanoma (A375), as well as other tumor types. In vivo two or three doses of CLR180099 showed similar or better activity to docetaxel in colorectal cancer. Additionally, CLR180099 demonstrated significantly improved survival benefit at both doses as compared to docetaxel. The tolerability assessment demonstrated that CLR180099 was well tolerated in both tumor bearing and normal animals and the FLV payload was toxic in both normal and tumor bearing mice. CLR180099 showed no toxic effects as compared to the FLV analogue payload alone demonstrating that this payload may benefit from targeted delivery with a phospholipid ether (PLE).

### Example 5. Synthesis of Compounds

Chemical synthesis steps were carried out as follows. Products were isolated using known techniques such as HPLC, and the resulting structures were verified by NMR and MS.

CLR2208 was synthesized according to Scheme 1. Scheme 1

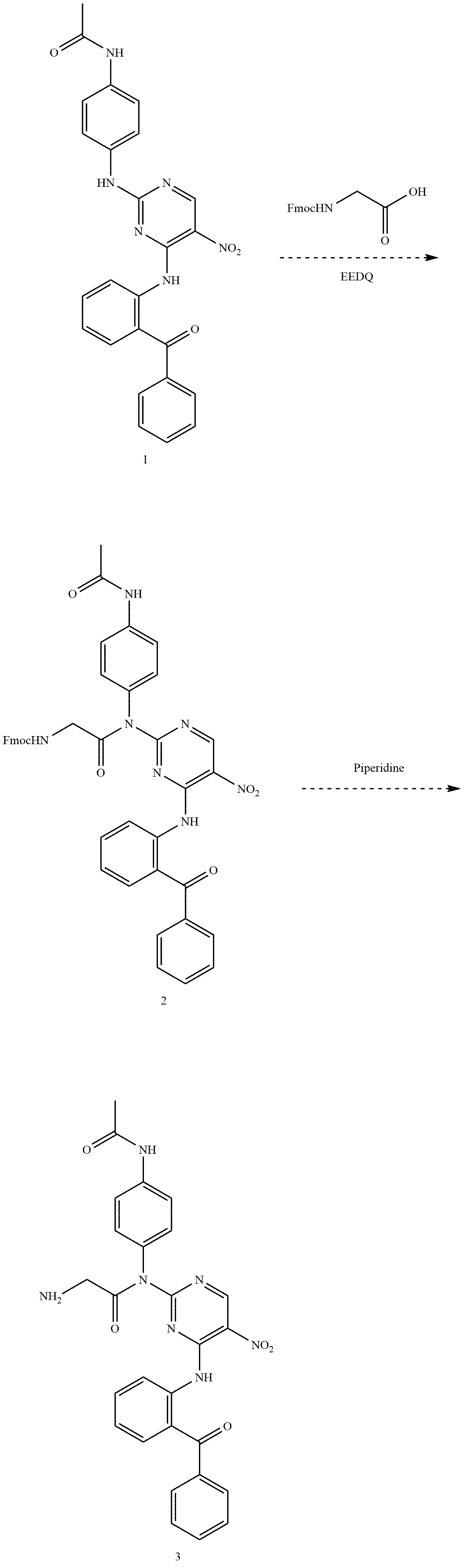

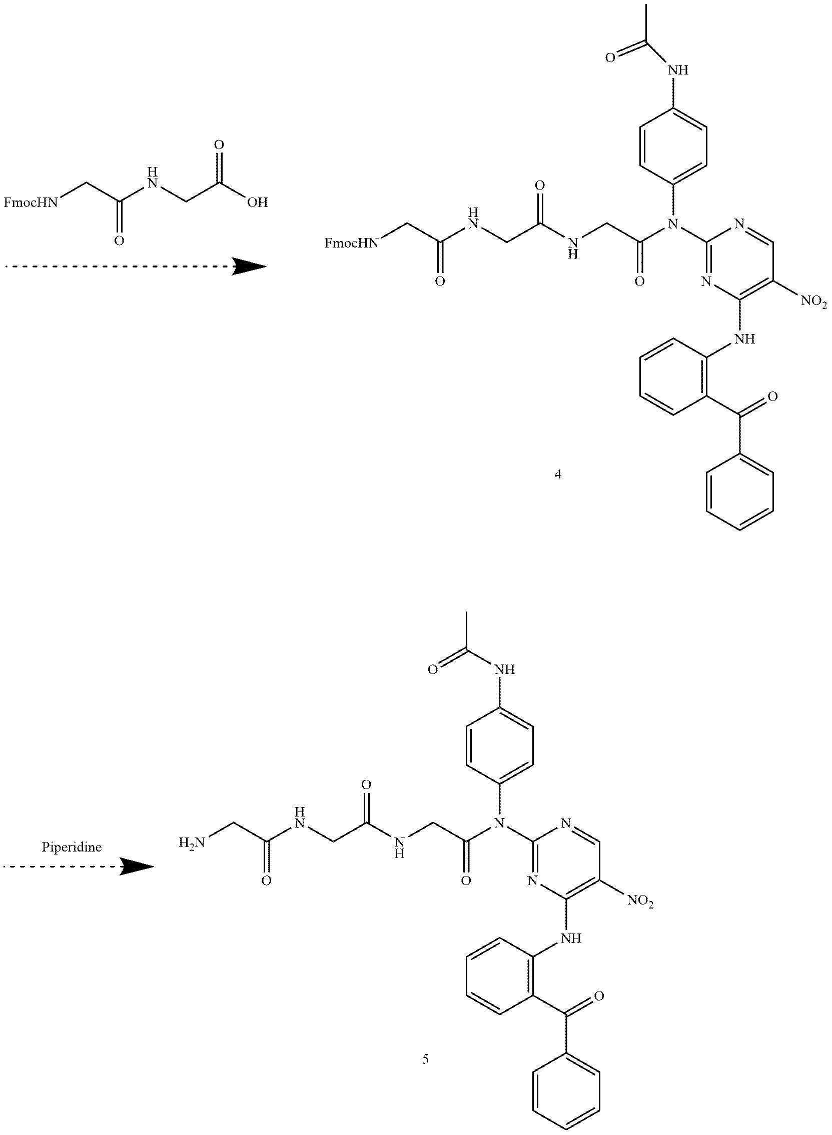

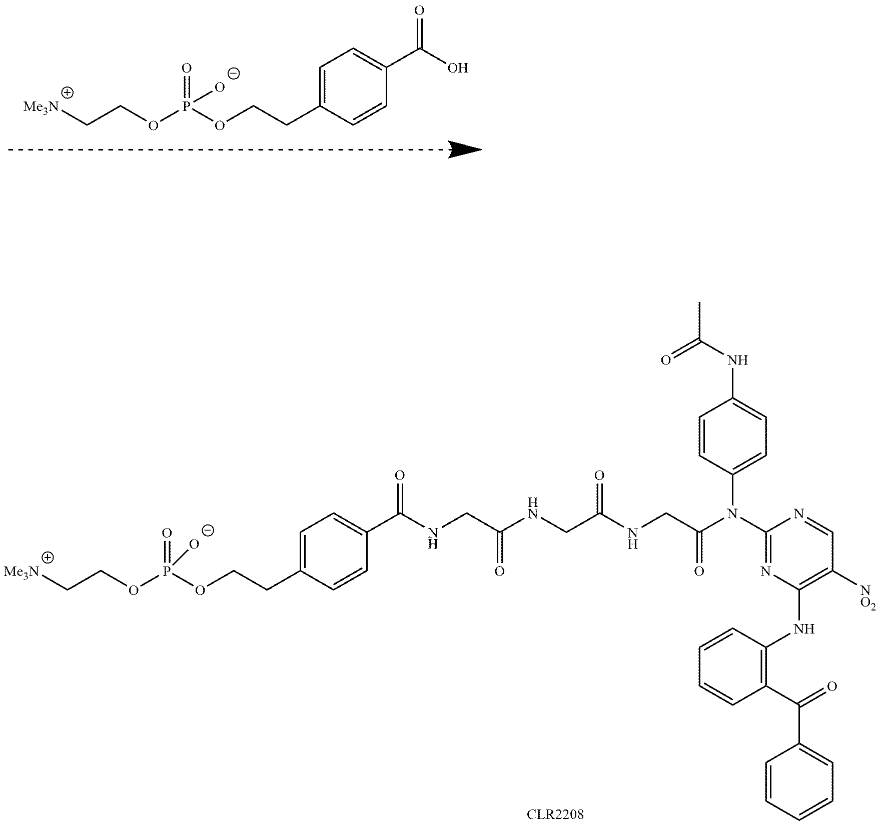

CLR2206 was synthesized according to Scheme 2. Hypophosphate chloride 1A (2.5 eq.) was used in Et₃N (10 eq.) and THF at -40° C. for 3 hours to prepare compound 2 from compound 1. Compound 2 was allowed to react with 2A (1 eq.) in Et₃N (1 eq.), CDI (1.5 eq.), ZnCl₂ (2.6 eq.), and DMF at 15° C. for 12 hours to yield compound 3. Deproptection of compound 3 in piperidine (5 eq. DMF, 15° C. for 3 hours) provided compound 4. Compounds 4 was allowed to react with 4A (1 eq.) in Et₃N (4 eq.), COMU (1.15 eq.), and CHCl₃ at 15° C. for 2 hours to yield CLR2206. Scheme 2

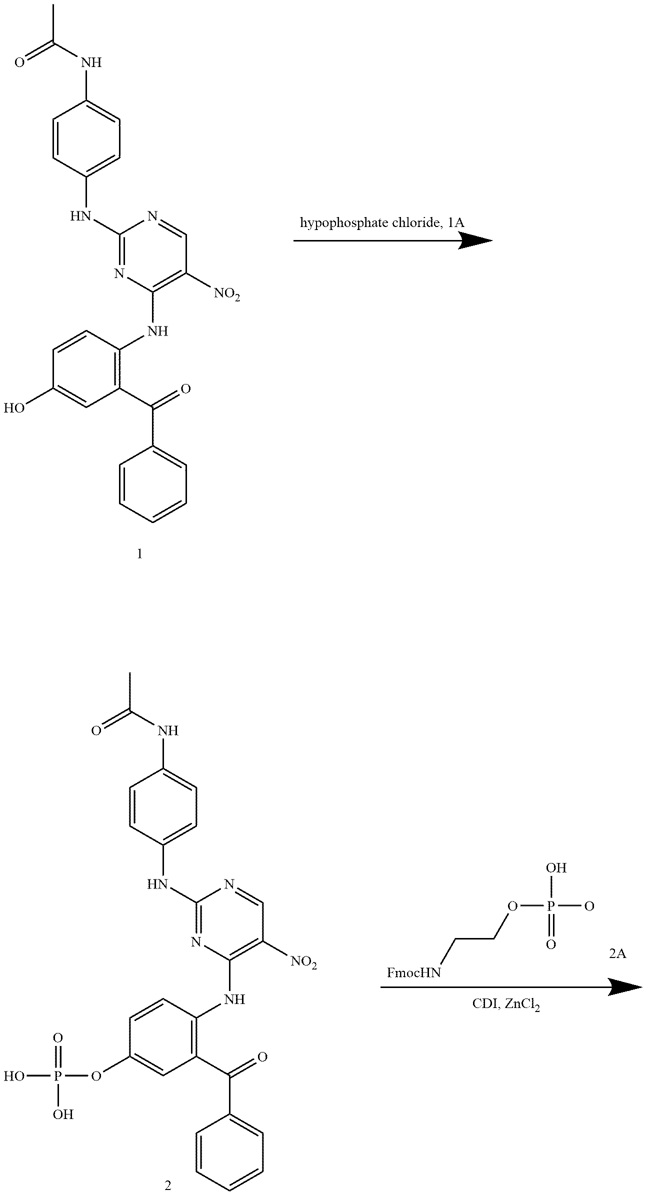

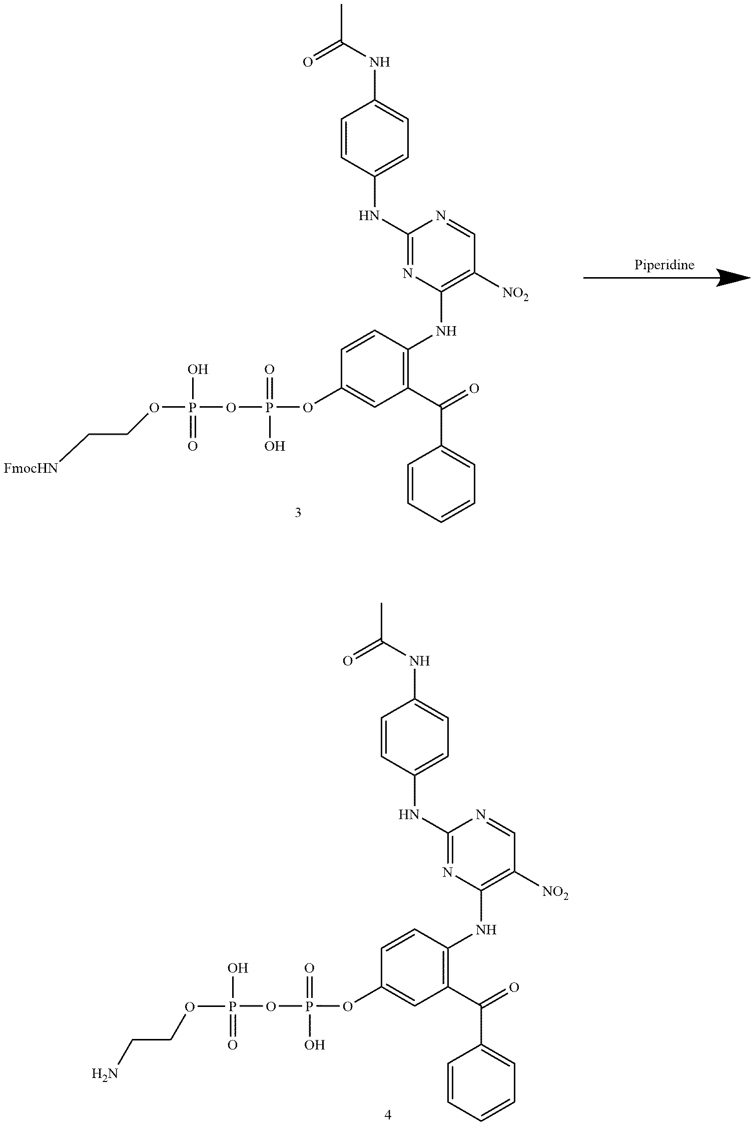

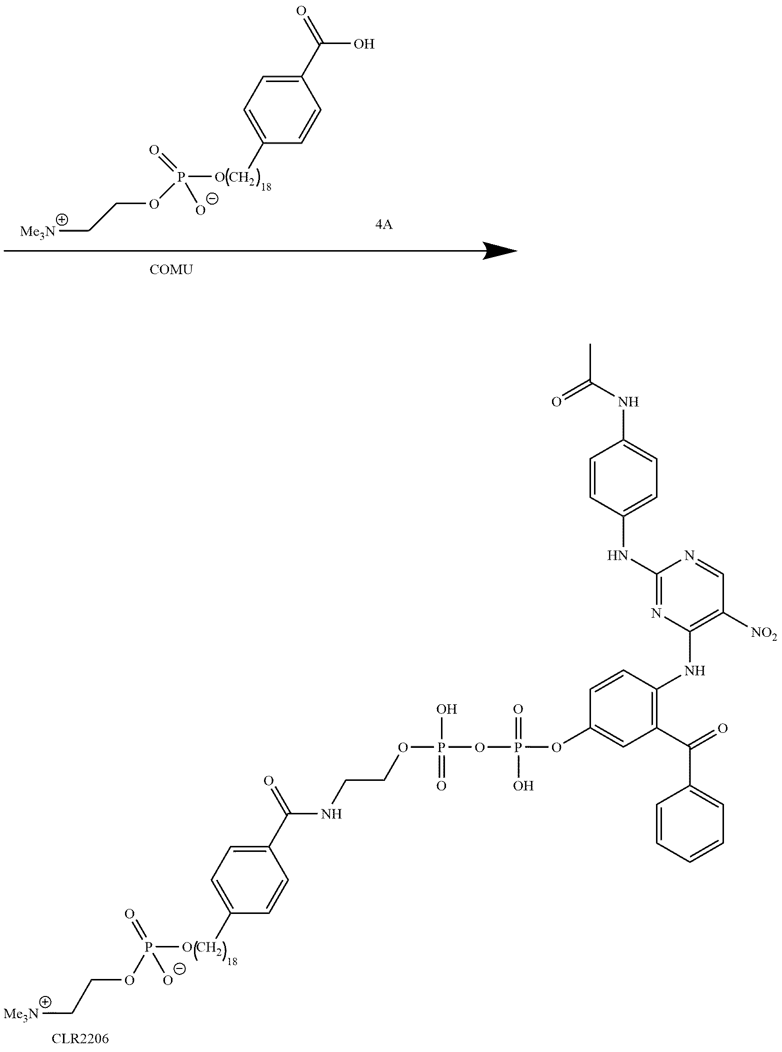

CLR2200 was synthesized according to Scheme 3. Compound 5 reacted with MMAE (0.8 eq.) in pyridine (Py, 20 eq.), HOBt (0.5 equ), and DMF at rt. for 12 hours to yield compound 6. Deproptection of compound 6 in piperidine (10 eq.) and DCM:AcN (1:1) at rt for 12 hours) provided compound 7. Compounds 7 was allowed to react with 7A (1 eq.), TEA (4.5 eq.), COMU (1.2 eq.), and CHCl₃ at rt for 12 hours to yield CLR2200. Scheme 3

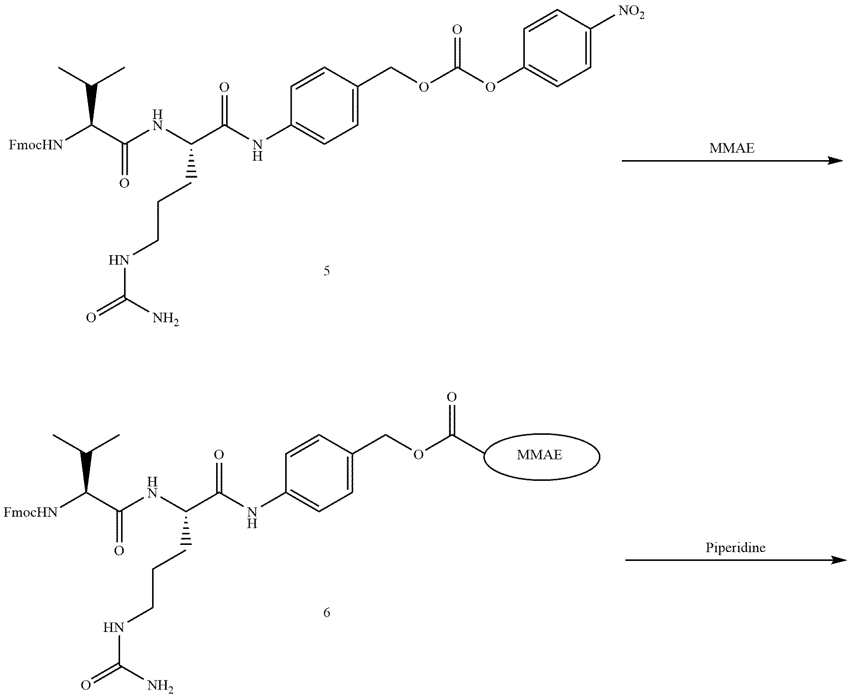

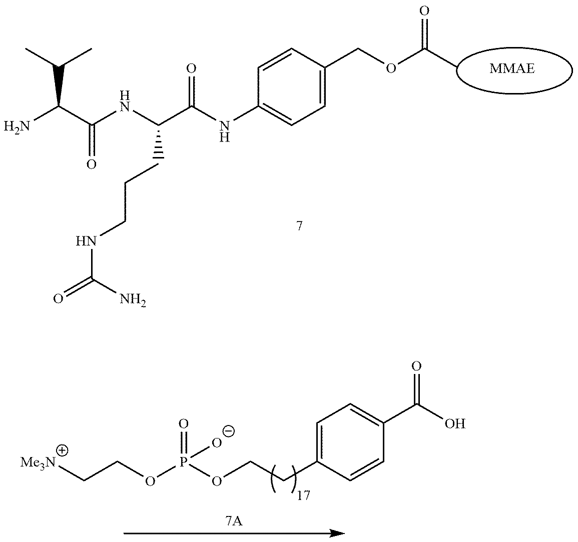

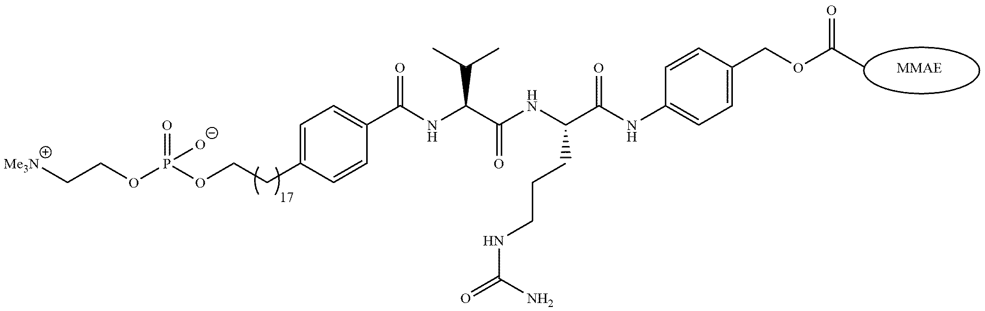

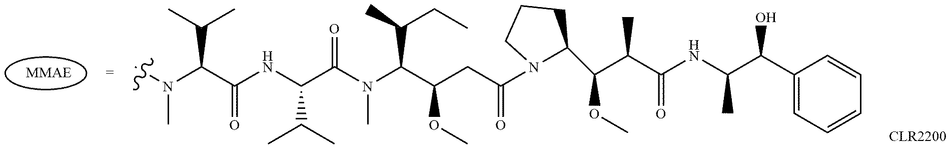

CLR200045 was prepared according to Scheme 4, or alternatively according to Scheme 5. Scheme 4

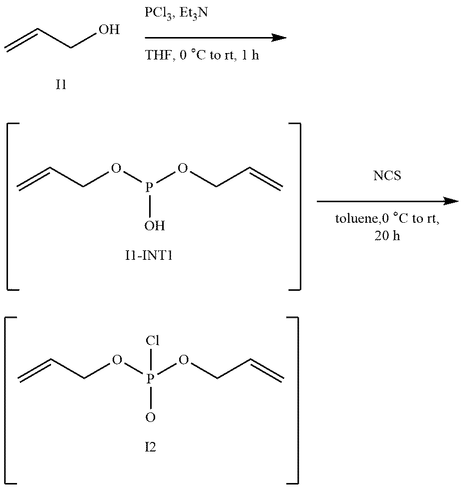

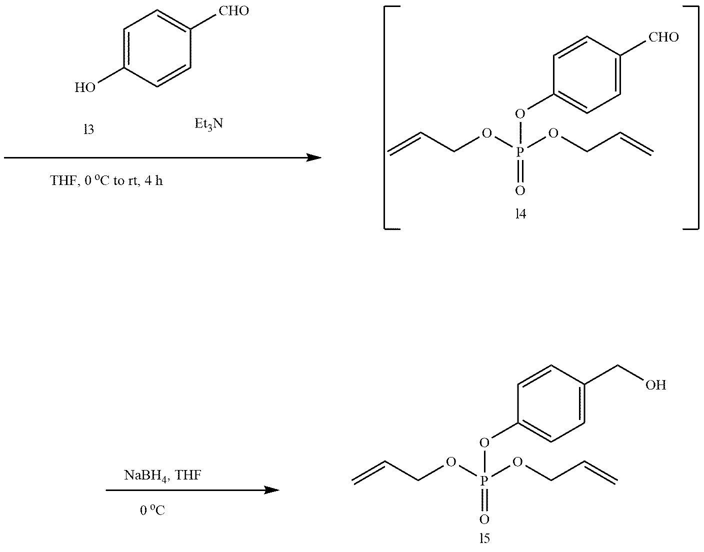

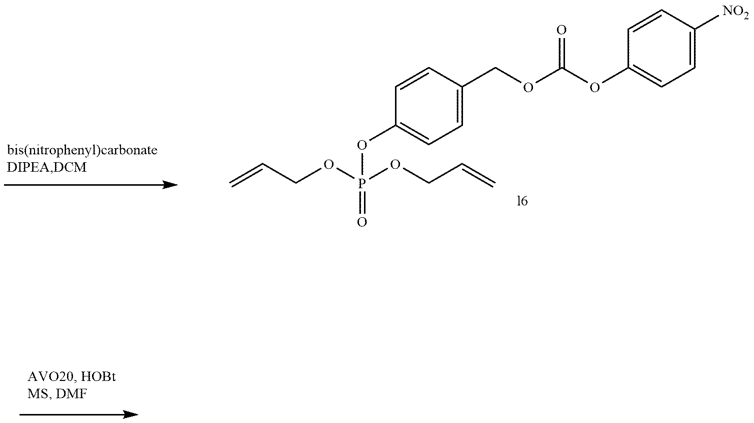

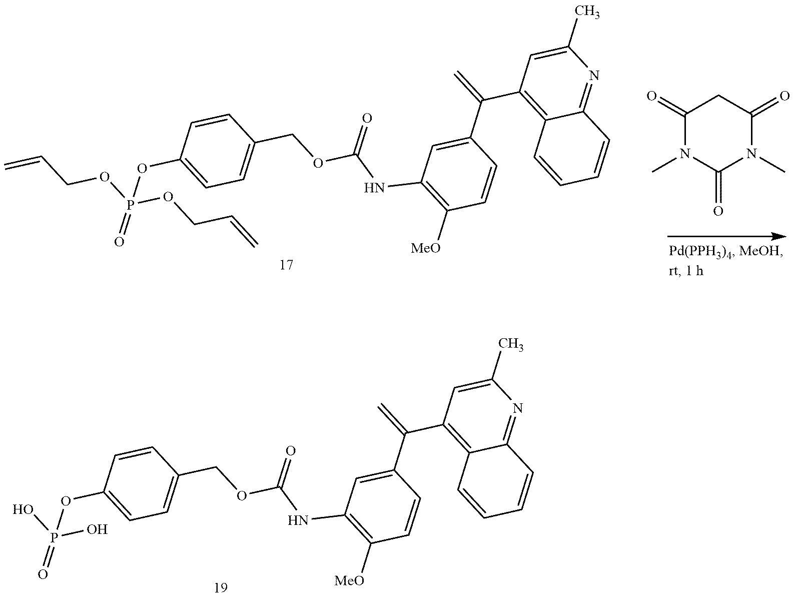

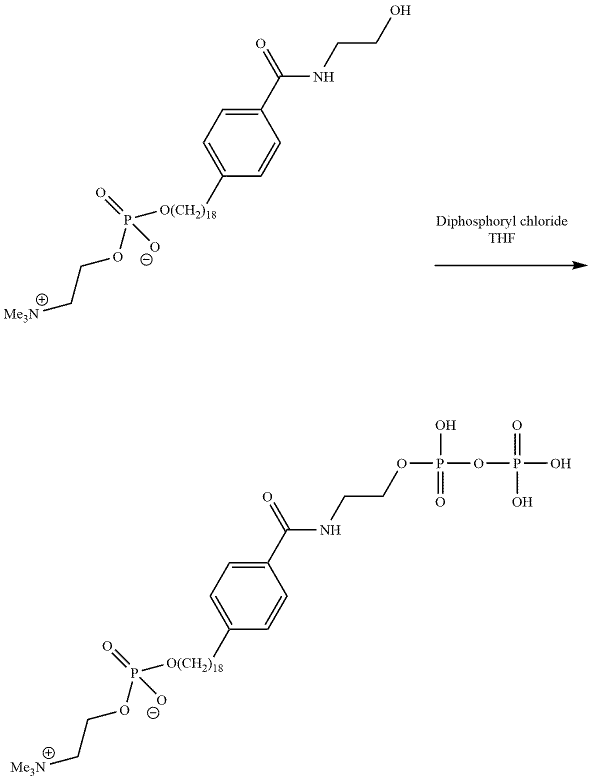

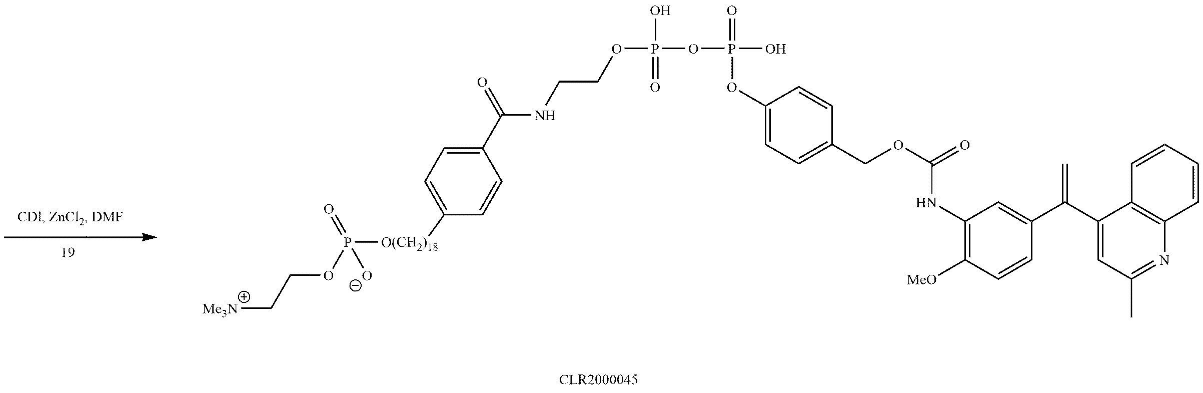

Scheme 5

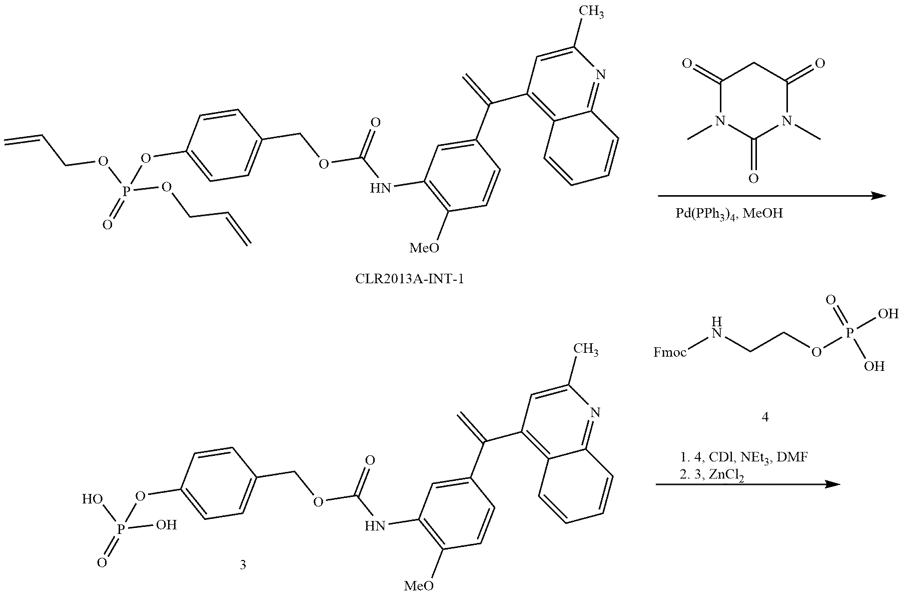

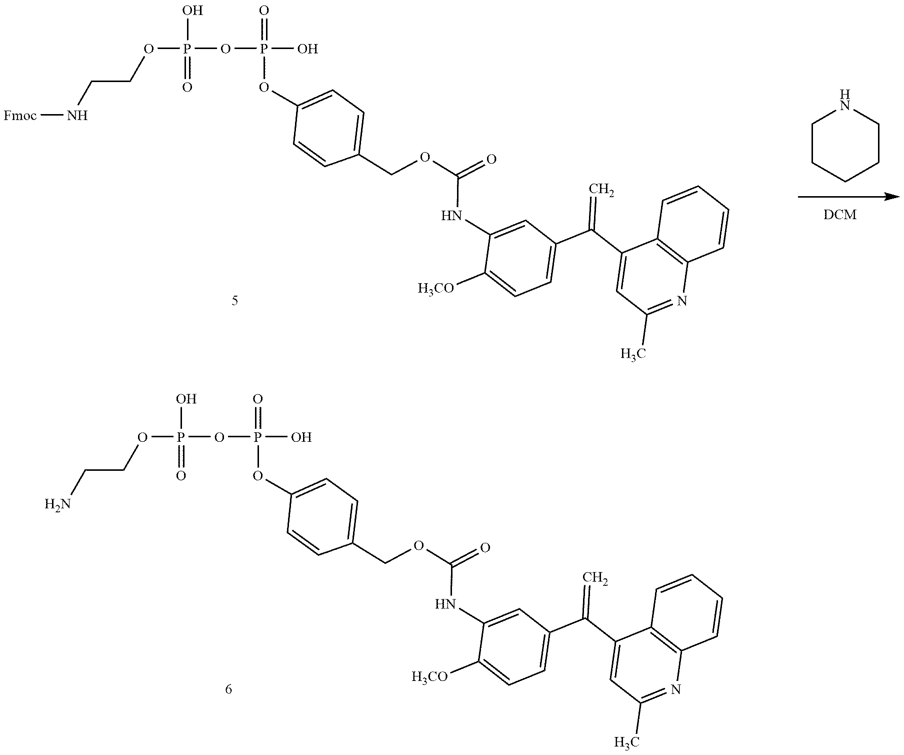

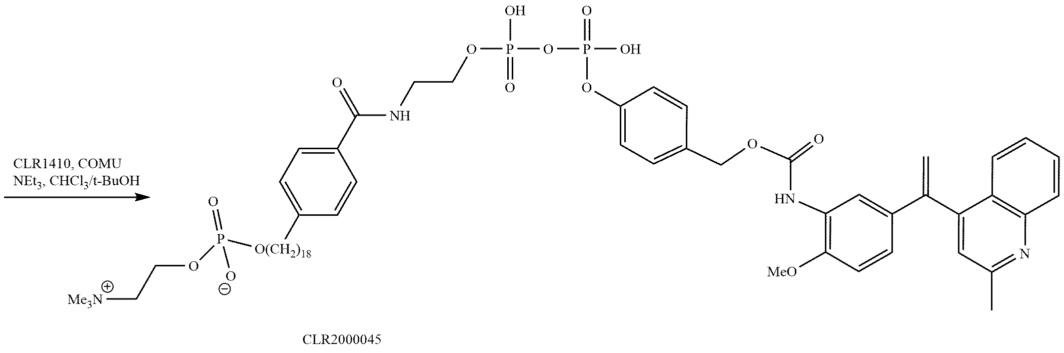

CLR2013 was prepared according to Scheme 6. Scheme 6

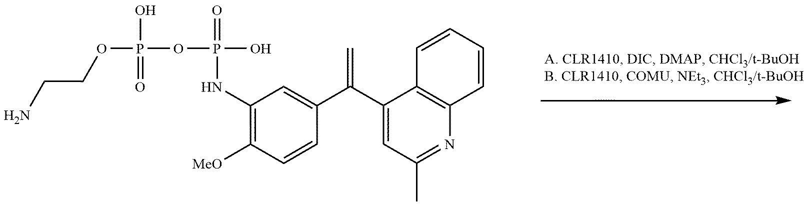

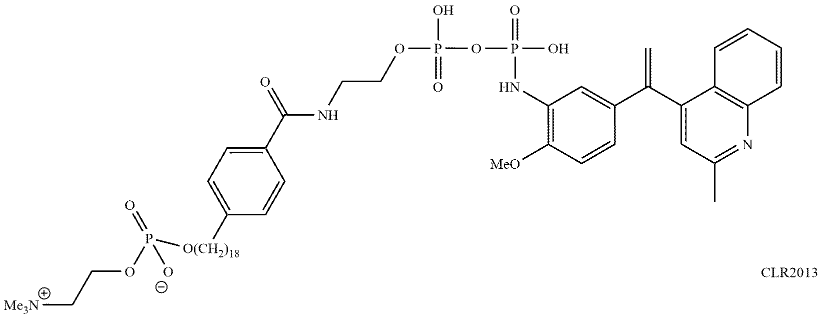

CLR1800095 was prepared according to Scheme 7. Scheme 7

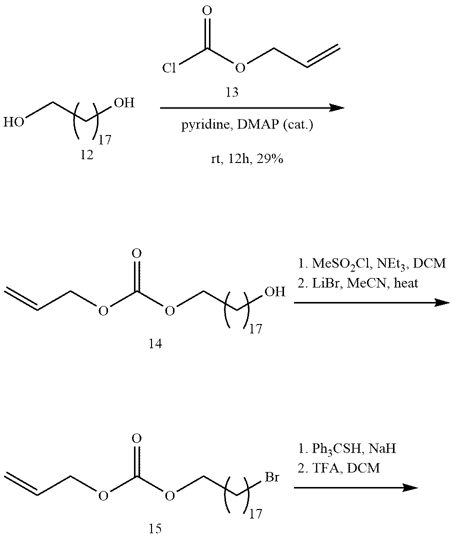

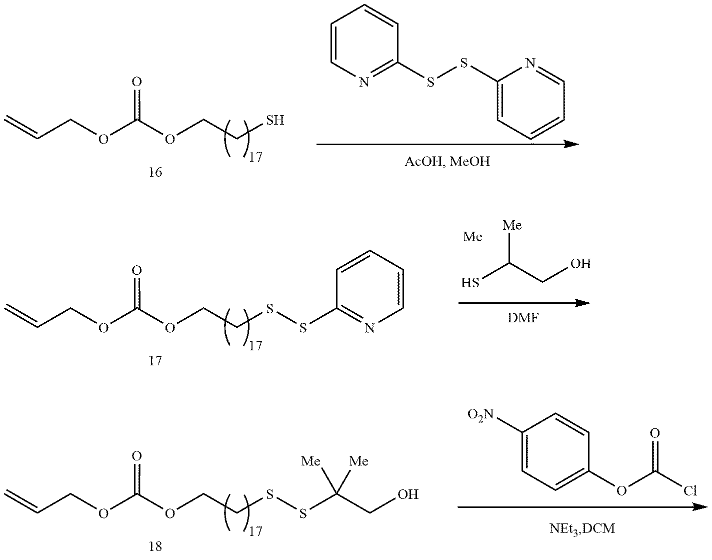

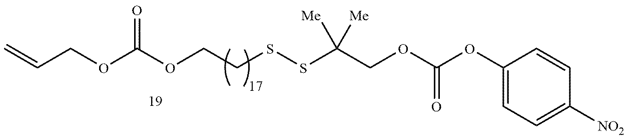

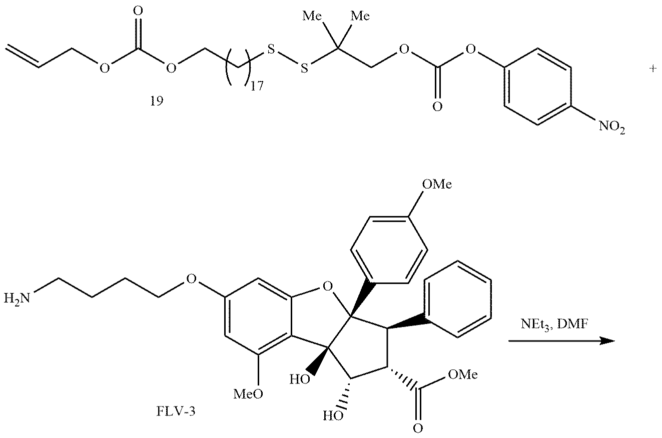

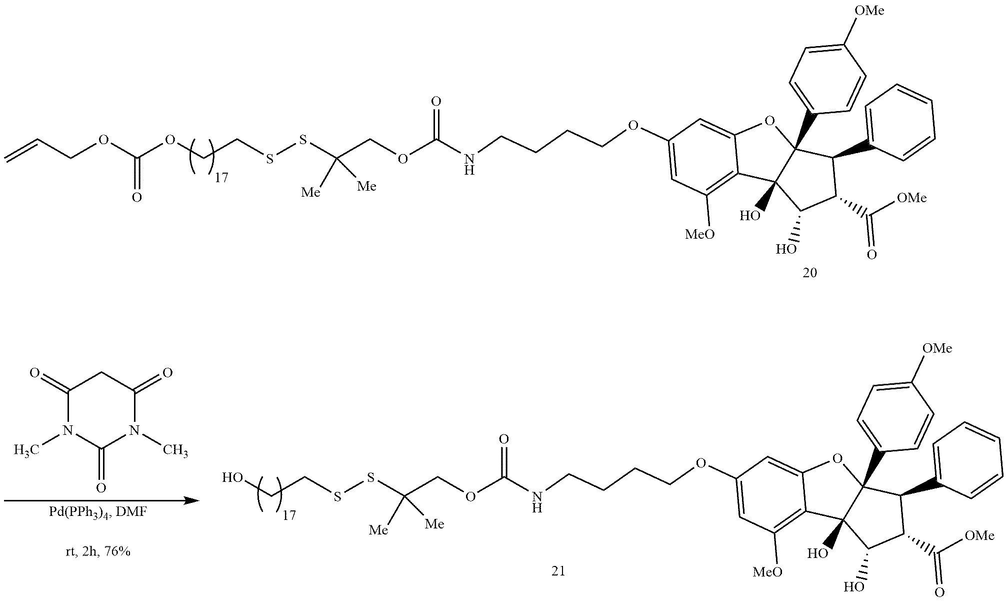

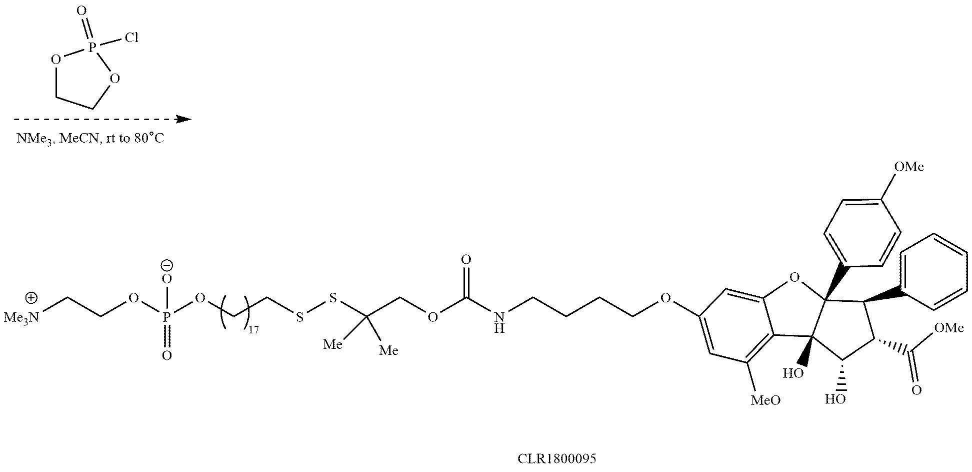

CLR180099B was prepared according to Scheme 8 (LCMS purity 97%). Scheme 8

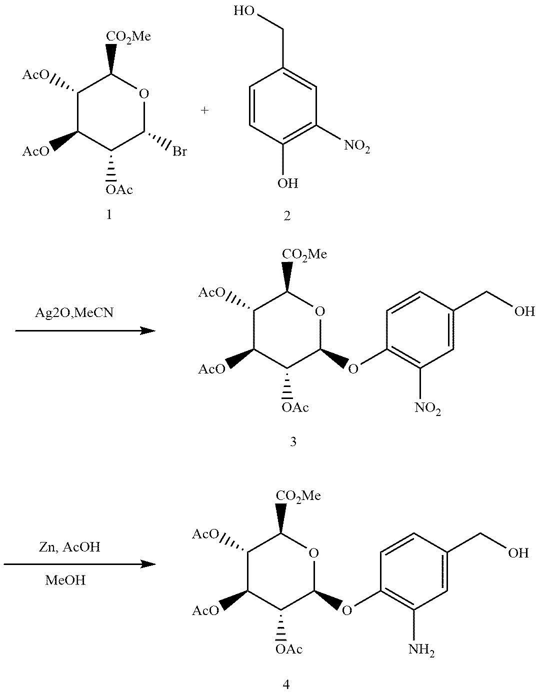

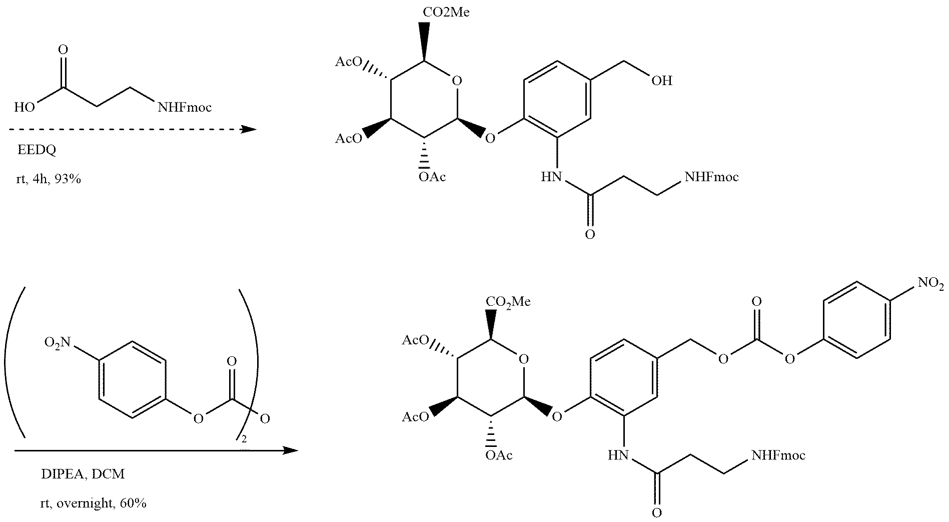

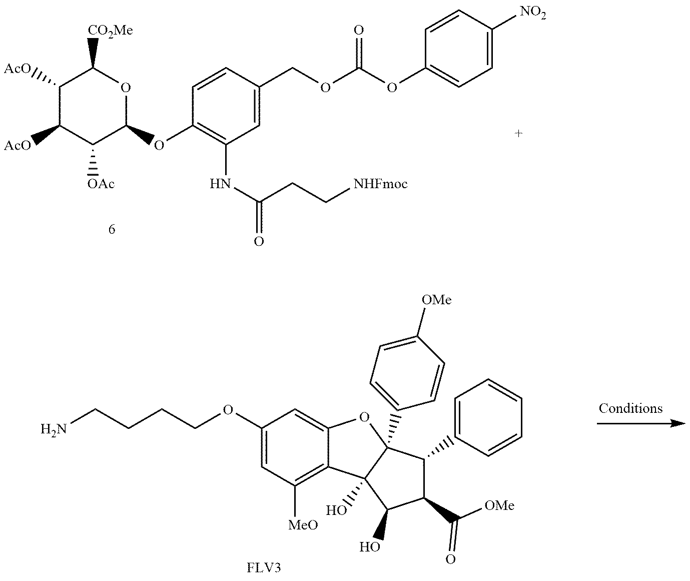

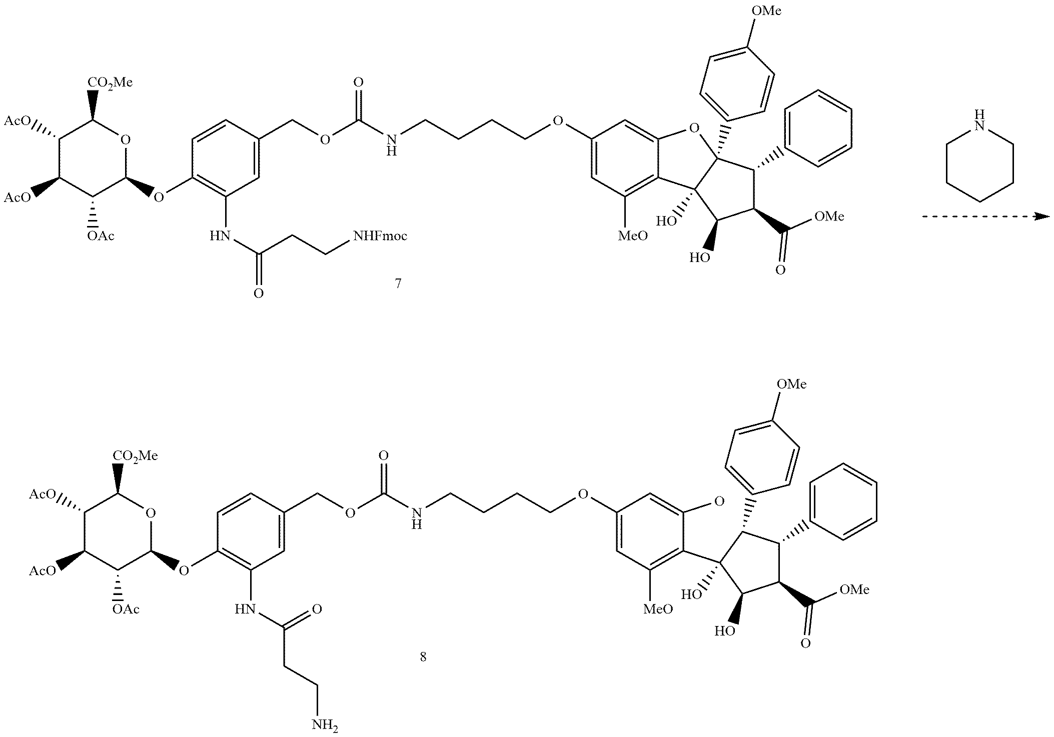

Conditions: NEt₃, DMF, rt, overnight; DIPEA, DCM, rt, overnight.

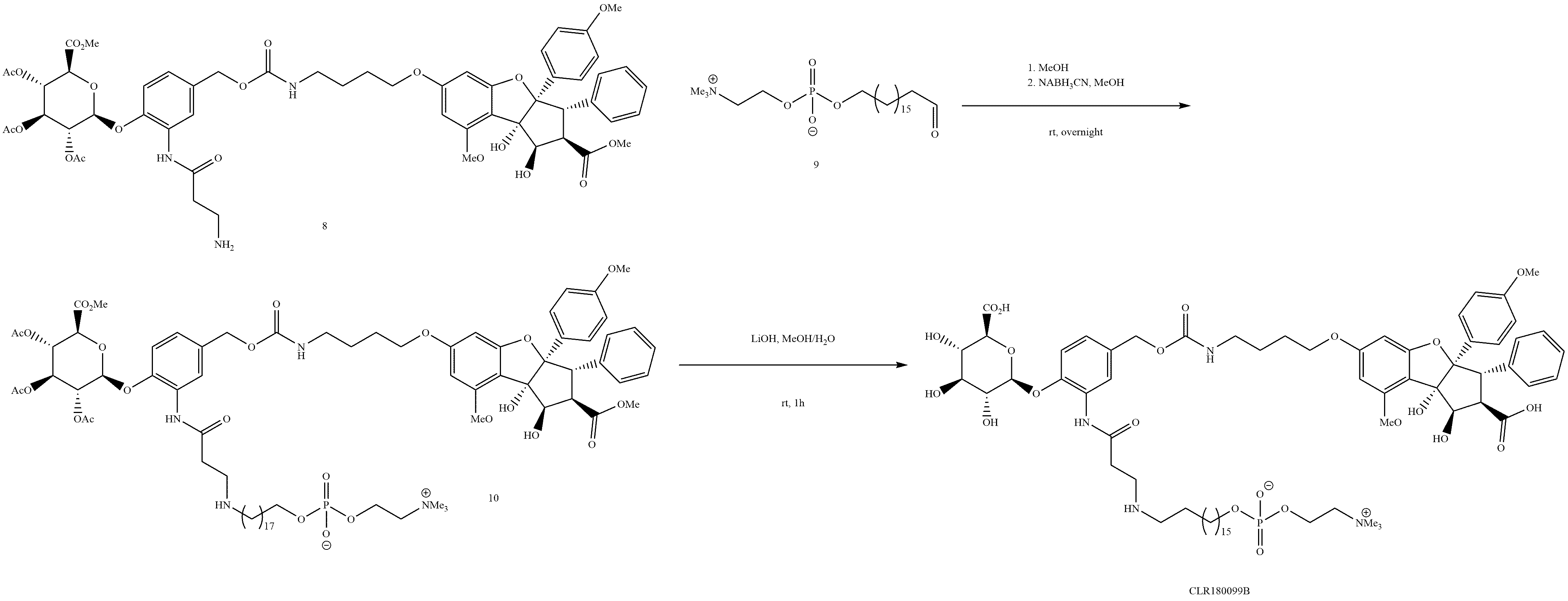

CLR180099A was prepared according to Scheme 9. Scheme 9

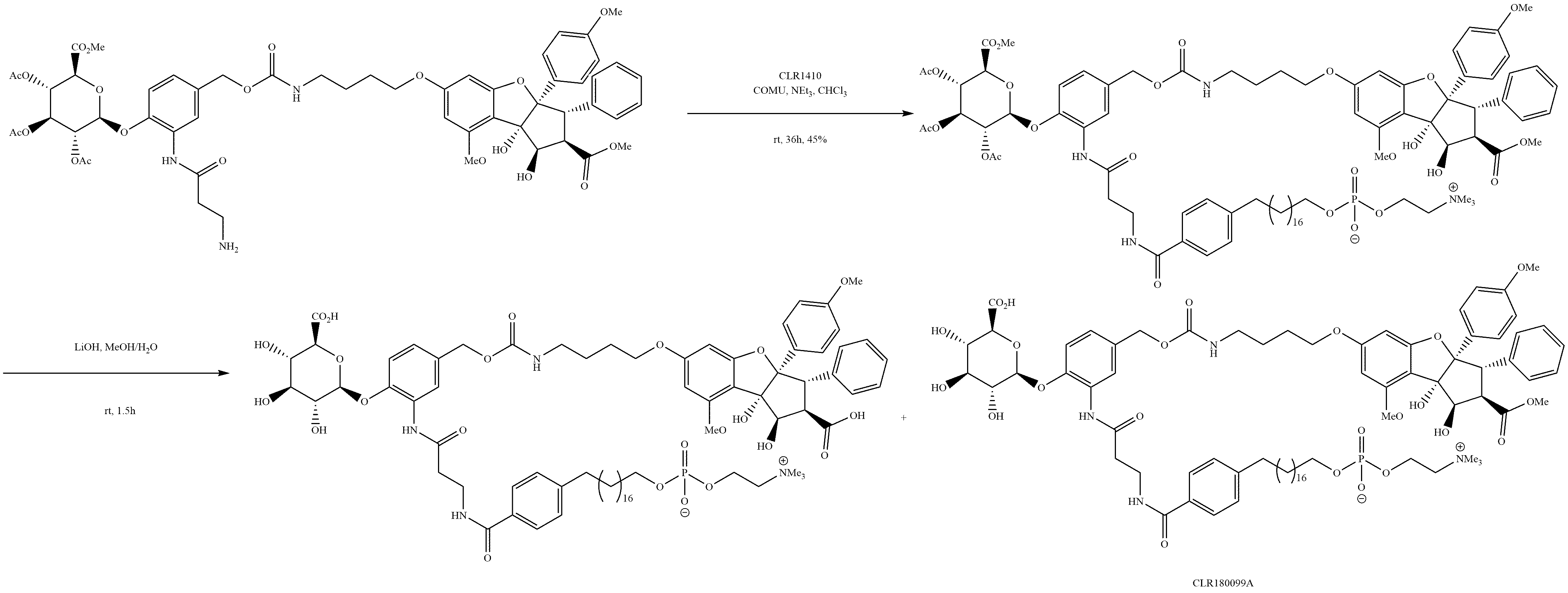

For reasons of completeness, various aspects of the invention are set out in the following numbered clauses:

Clause 1. A compound of formula (I), or a pharmaceutically acceptable salt thereof,

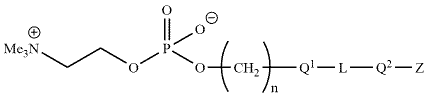

wherein n is 2-20;

Q¹ is a bond or

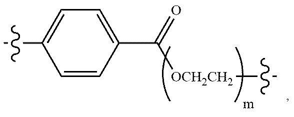

wherein m is 0-100; L is

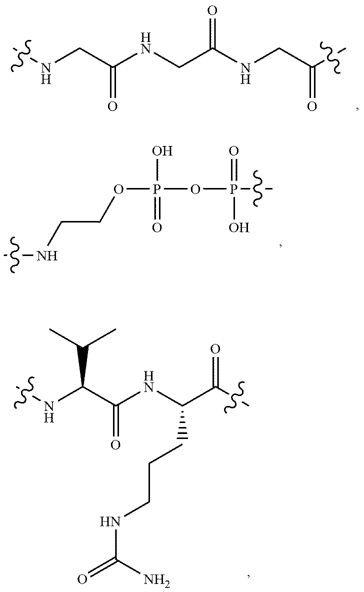

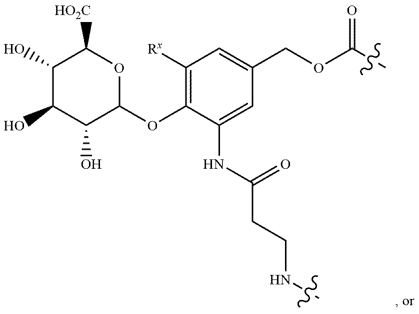

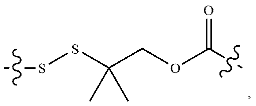

wherein R^{x} is H or halogen; Q² is a bond or a self-immolative spacer; and Z is an anti-cancer drug.

Clause 2. The compound of clause 1, or a pharmaceutically acceptable salt thereof, wherein Q¹ is a bond or

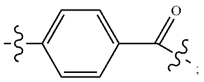

L-Q² is

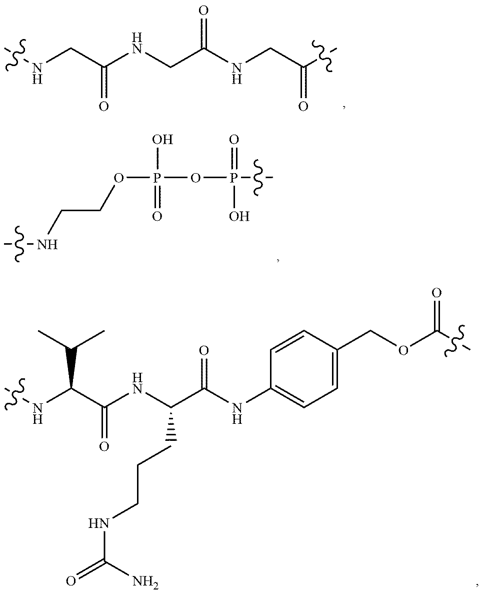

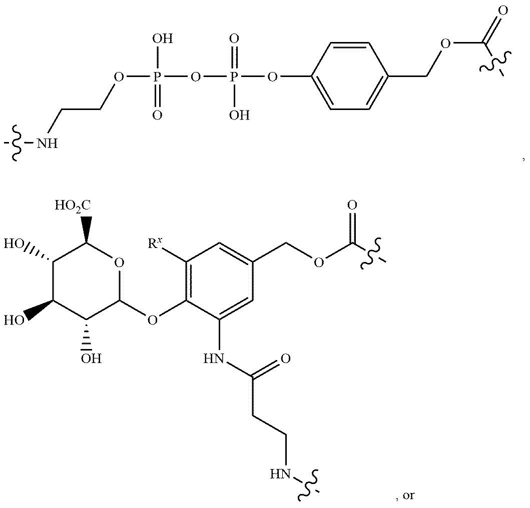

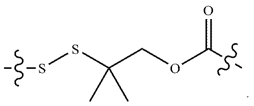

Clause 3. The compound of any one of clauses 1-2, or a pharmaceutically acceptable salt thereof, wherein Z is a polo-like kinase 1 (PLK-1) inhibitor, a tubulin polymerase inhibitor, a tubulin stabilizer, an antineoplastic agent, an eukaryotic translation initiation factor 4 (EIF4) inhibitor, a combretastatin A-4 analog, or a flavagline analog.

Clause 4. The compound of any one of clauses 1-3, having a structure of formula (l-a), or a pharmaceutically acceptable salt thereof, wherein Q¹ is

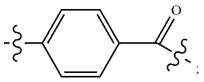

L-Q² is

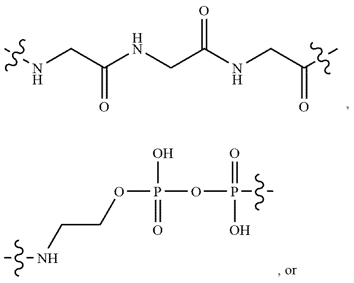

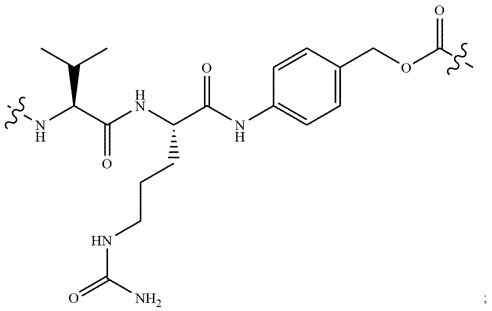

and

Z is a PLK-1 inhibitor, a tubulin polymerase inhibitor, a tubulin stabilizer, an antineoplastic agent, or an eukaryotic translation initiation factor 4 (EIF4) inhibitor.

Clause 5. The compound of clause 4, wherein Z is a PLK-1 inhibitor or an antineoplastic agent selected from the group consisting of monomethyl auristatin E (MMAE), monomethyl auristatin F (MMAF), and monomethyl auristatin E (MMAD).

Clause 6. The compound of any one of clauses 1-3, having a structure of formula (l-b), or a pharmaceutically acceptable salt thereof, wherein n is 18; Q¹ is

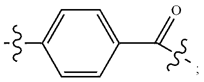

L-Q² is

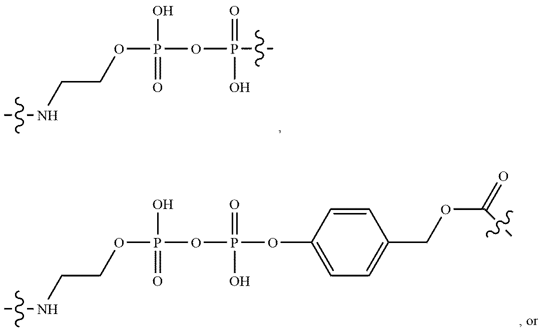

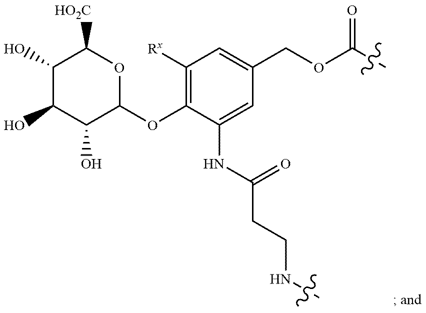

Z is a combretastatin A-4 analog.

Clause 7. The compound of any one of clauses 1-3, having a structure of formula (l-c), or a pharmaceutically acceptable salt thereof, wherein n is 18; Q¹ is a bond or

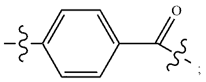

L-Q² is

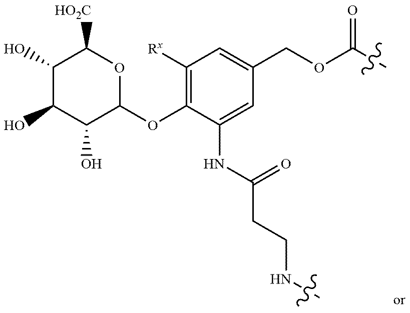

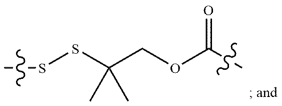

Z is a flavagline analog.

Clause 8. The compound of clause 1, which is selected from the group consisting of

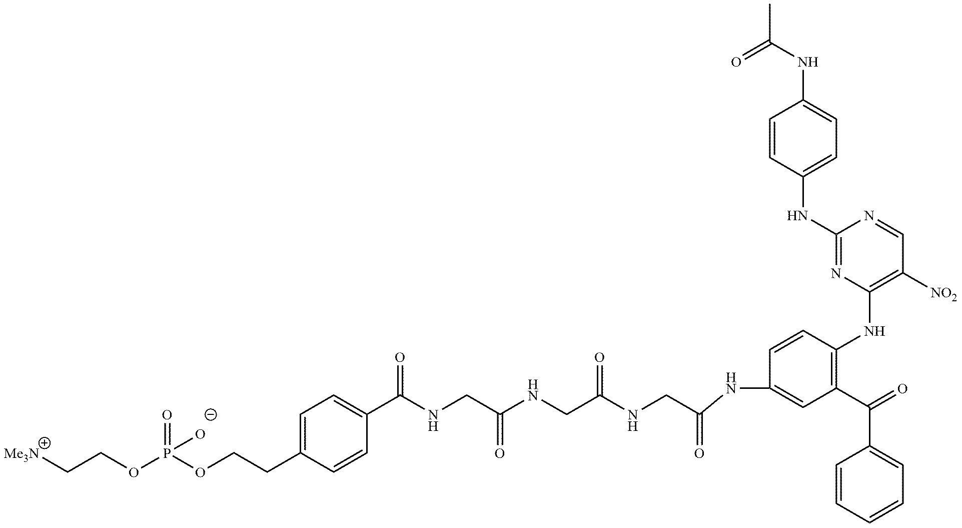

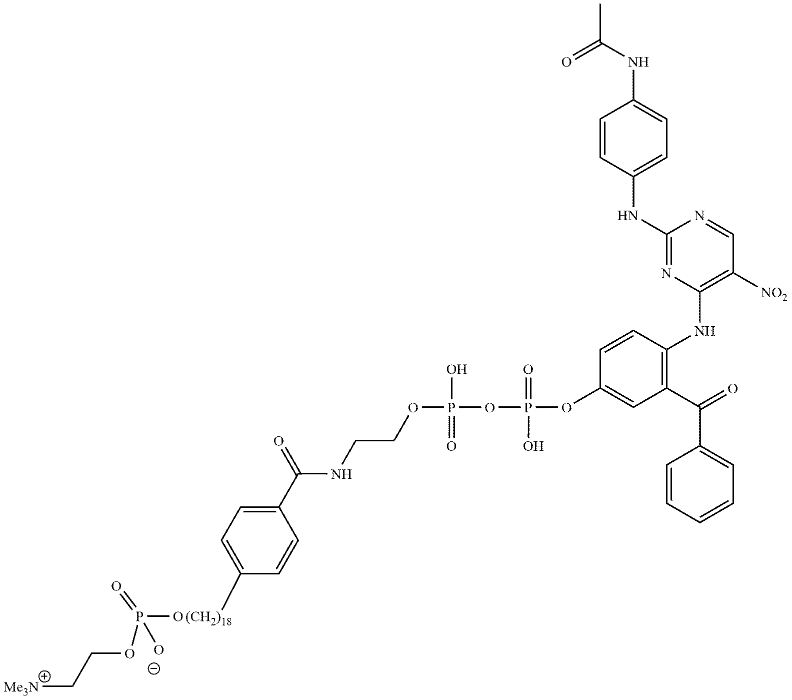

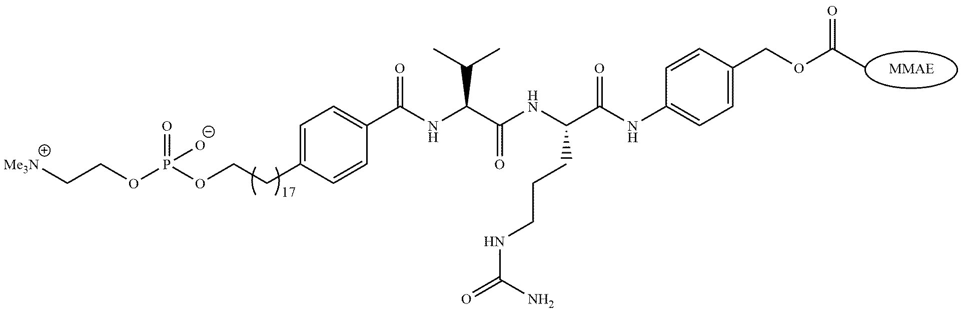

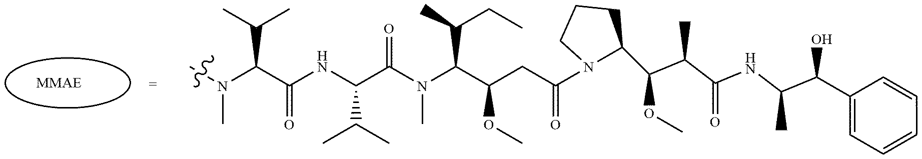

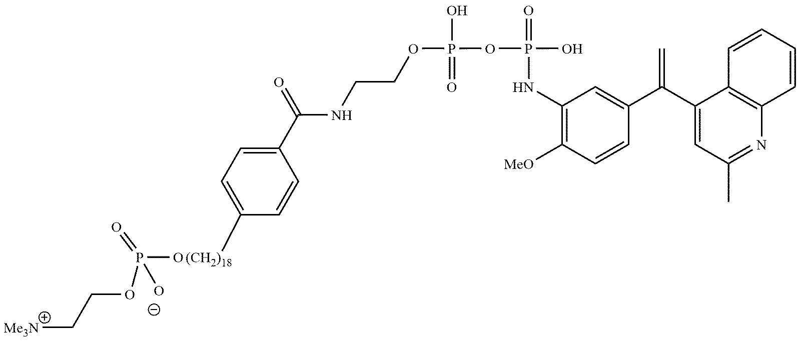

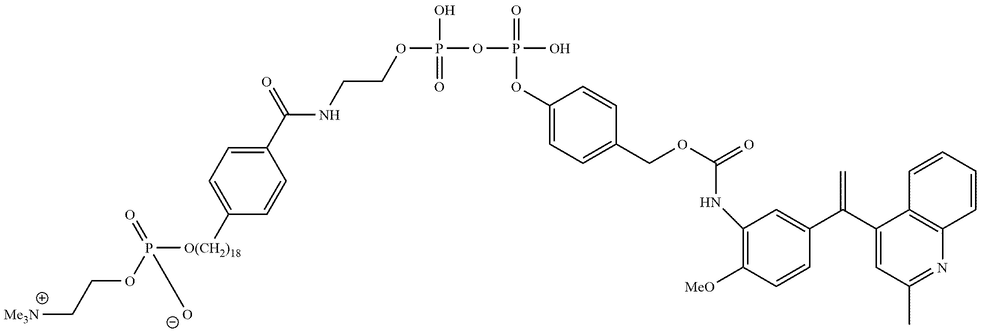

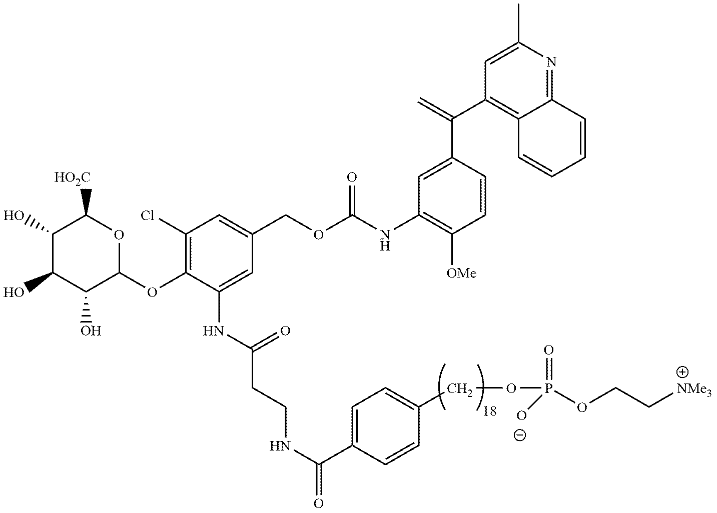

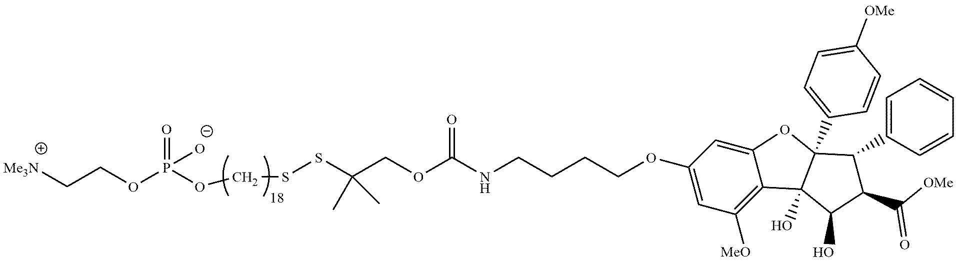

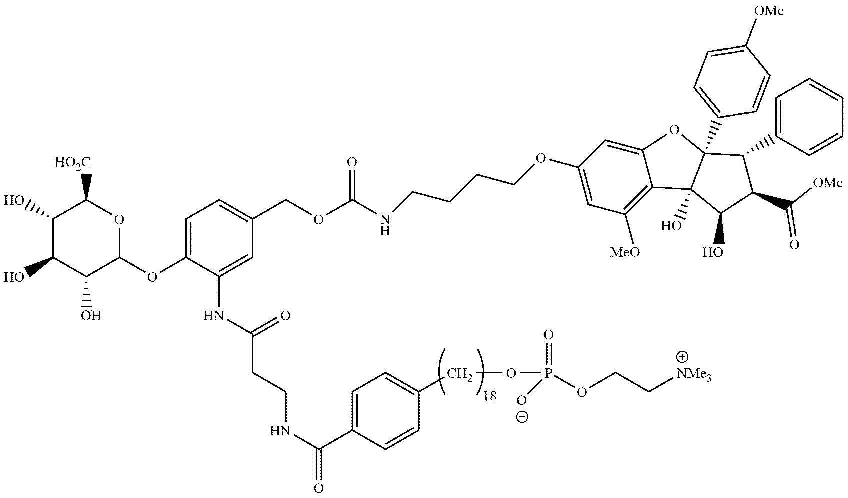

and

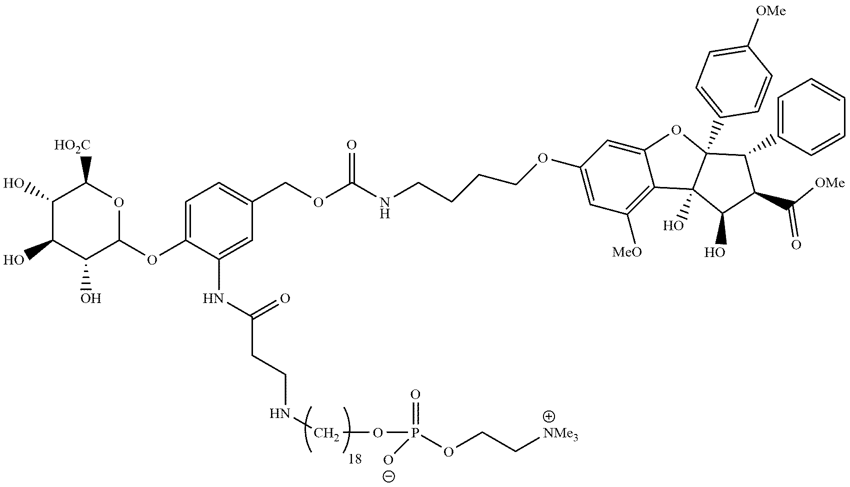

or a pharmaceutically acceptable salt thereof.

Clause 9. A pharmaceutical composition comprising a compound of any one of clauses 1-8, or a pharmaceutically acceptable salt thereof, and a pharmaceutically acceptable carrier.

Clause 10. A method of treating cancer in a subject in need thereof, comprising administering an effective amount of a compound of any one of clauses 1-8, or a pharmaceutically acceptable salt thereof.

Clause 11. The method of clause 10, wherein the cancer is melanoma, lung cancer, colorectal cancer, breast cancer, or a combination thereof.

Clause 12. The method of any one of clauses 10-11, wherein
the lung cancer comprises small cell lung cancer, non-small cell lung cancer, or a combination thereof;
the melanoma comprises superficial spreading melanoma, nodular melanoma, lentigo maligna melanoma, acral lentiginous melanoma, amelanotic melanoma, nevoid melanoma, spitzoid melanoma, desmoplastic melanoma, or a combination thereof;
the colorectal cancer comprises adenocarcinoma; or
the breast cancer comprises invasive breast ductal carcinoma, metastatic breast cancer, inflammatory breast cancer, triple negative breast cancer, ductal carcinoma in situ, or a combination thereof.

Clause 13. The method of any one of clauses 10-12, wherein the cancer comprises cancer stem cells.

Clause 14. The method of any one of clauses 10-13, wherein the cancer comprises metastatic cancer cells

Clause 15. The method of any one of clauses 10-14, wherein the cancer comprises circulating tumor cells.

Clause 16. The method of any one of clauses 10-15, wherein the cancer is melanoma, lung cancer, colorectal cancer, or a combination thereof, and wherein the compound is a compound of formula (I-a), or a pharmaceutically acceptable salt thereof.

Clause 17. The method of any one of clauses 10-15, wherein the cancer is breast cancer, wherein the subject (1) is estrogen receptor positive, (2) is both estrogen receptor negative and progesterone receptor negative, (3) expresses HER2 (HER2+), (4) does not express HER2 (HER2-), or a combination thereof.

Clause 18. The method of any one of clauses 10-15 and 17, wherein the cancer is breast cancer, and wherein the compound is a compound of formula (l-b),or a pharmaceutically acceptable salt thereof.

Clause 19. The method of any one of clauses 10-15, wherein the cancer is cancer is melanoma, lung cancer, colorectal cancer, breast cancer, or a combination thereof, and wherein the compound is a compound of formula (l-c),or a pharmaceutically acceptable salt thereof.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A compound of formula (I), or a pharmaceutically acceptable salt thereof, 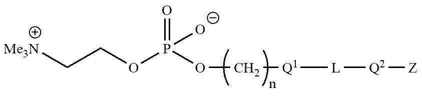
wherein
n is 2-20;
Q¹ is a bond or 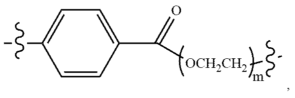
wherein m is 0-100;
L is 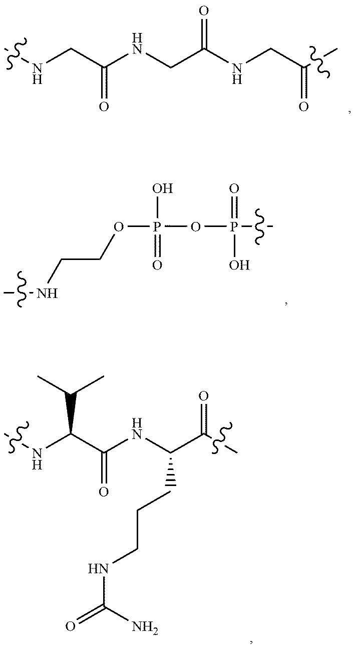 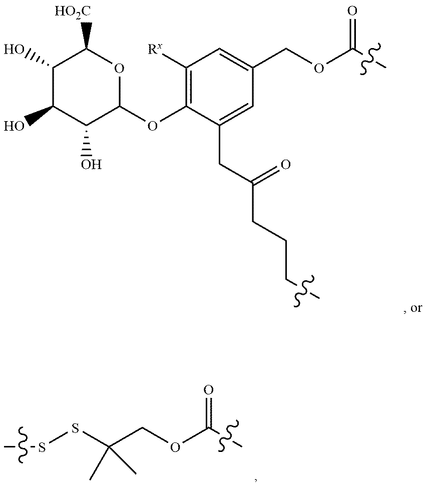
wherein R
^{x} is H or halogen;
Q² is a bond or a self-immolative spacer; and
Z is an anti-cancer drug.

2. The compound of claim 1, or a pharmaceutically acceptable salt thereof, wherein
Q¹ is a bond or 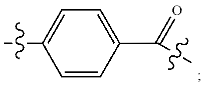 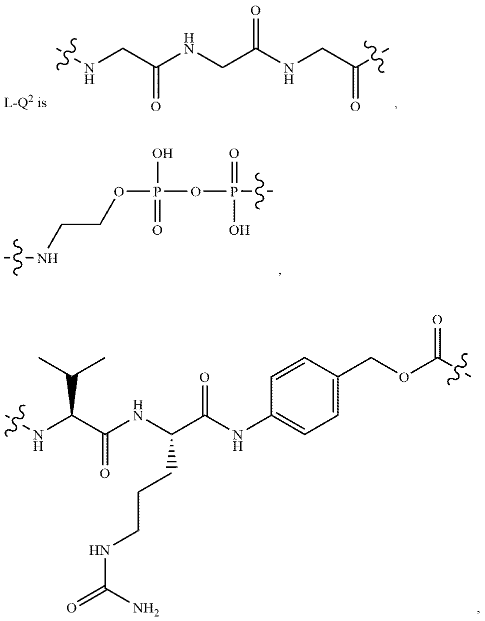 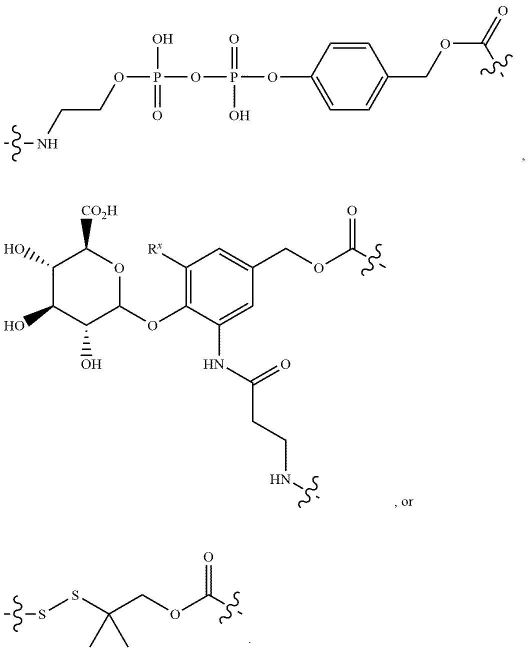
.

3. The compound of claim 1, or a pharmaceutically acceptable salt thereof, wherein Z is a polo-like kinase 1 (PLK-1) inhibitor, a tubulin polymerase inhibitor, a tubulin stabilizer, an antineoplastic agent, an eukaryotic translation initiation factor 4 (EIF4) inhibitor, a combretastatin A-4 analog, or a flavagline analog.

4. The compound of claim 1, having a structure of formula (I-a), or a pharmaceutically acceptable salt thereof, wherein
Q¹ is 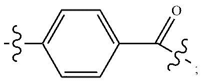
L-Q² is and 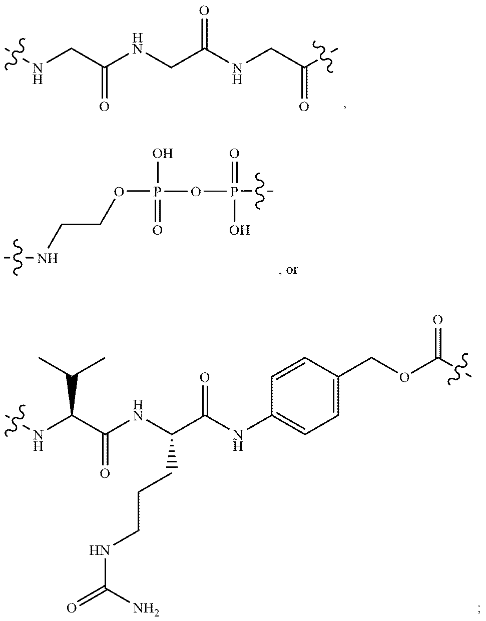
Z is a PLK-1 inhibitor, a tubulin polymerase inhibitor, a tubulin stabilizer, an antineoplastic agent, or an eukaryotic translation initiation factor 4 (EIF4) inhibitor.

5. The compound of claim 4, wherein Z is a PLK-1 inhibitor or an antineoplastic agent selected from the group consisting of monomethyl auristatin E (MMAE), monomethyl auristatin F (MMAF), and monomethyl auristatin E (MMAD).

6. The compound of claim 1, having a structure of formula (I-b), or a pharmaceutically acceptable salt thereof, wherein
n is 18;
Q¹ is 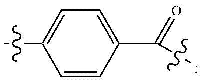
L-Q² is 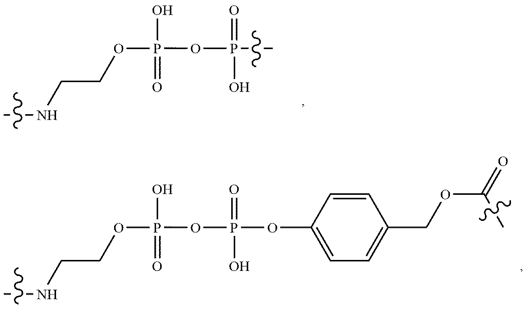
or 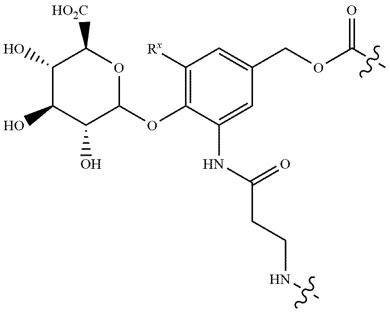
and
Z is a combretastatin A-4 analog.

7. The compound of claim 1, having a structure of formula (I-c), or a pharmaceutically acceptable salt thereof, wherein
n is 18;
Q¹ is a bond or 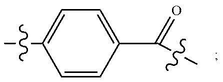
L-Q² is 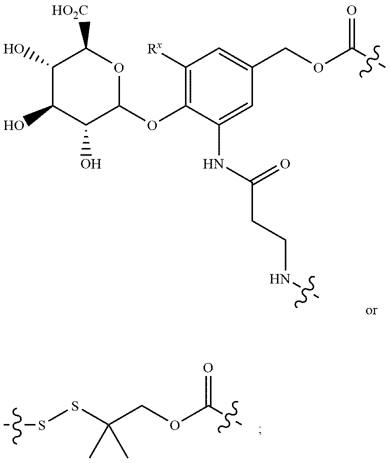
and
Z is a flavagline analog.

8. The compound of claim 1, which is selected from the group consisting of 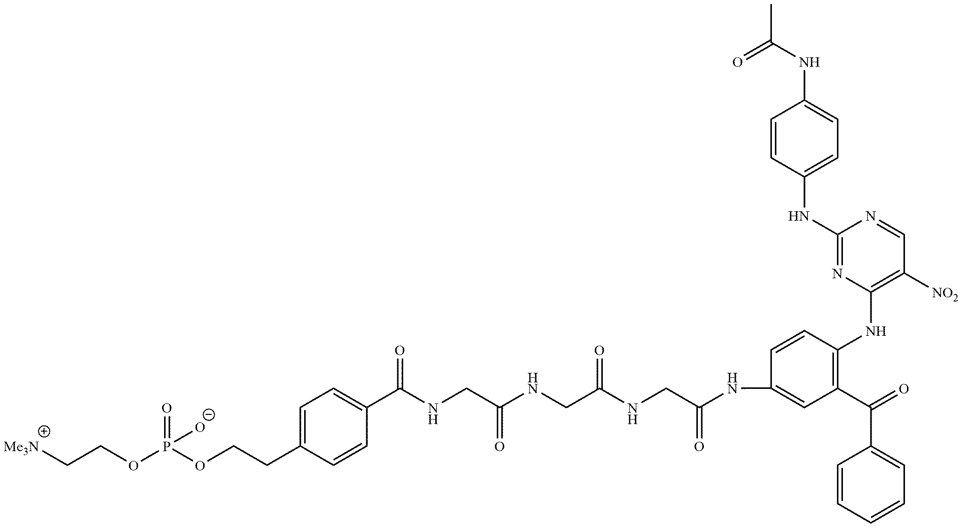 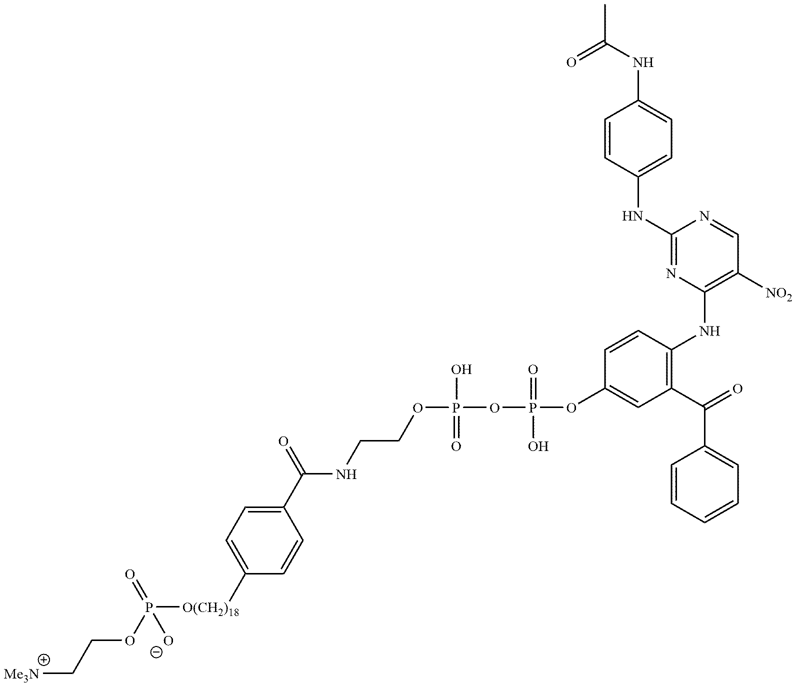 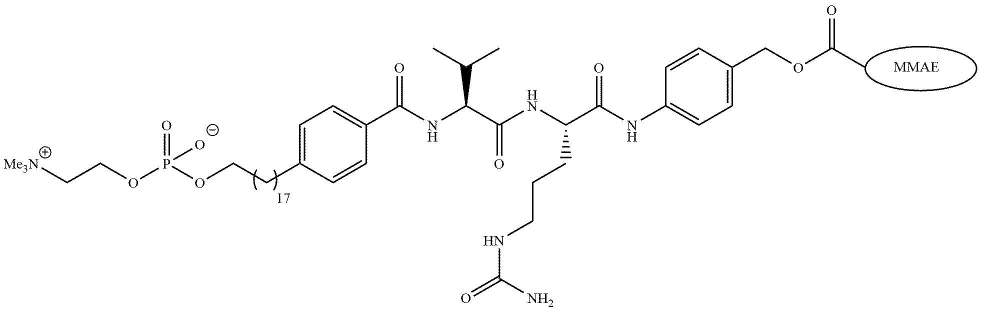 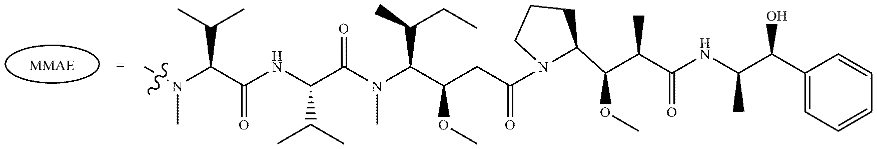 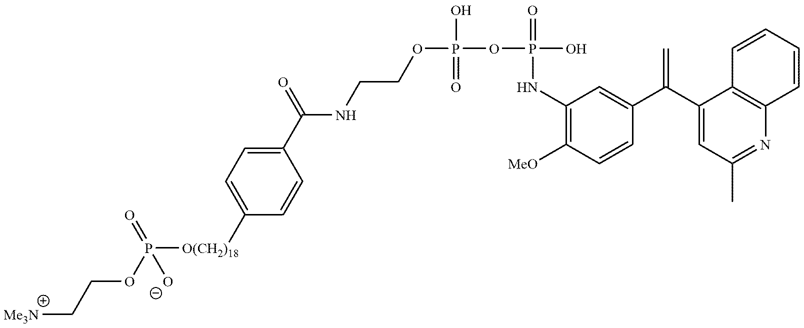 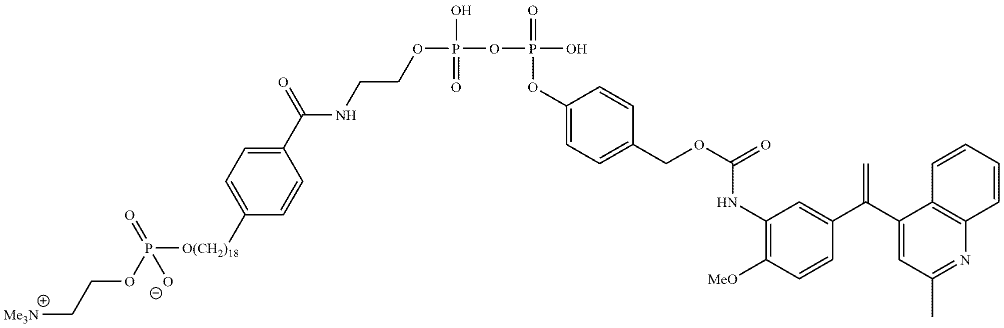 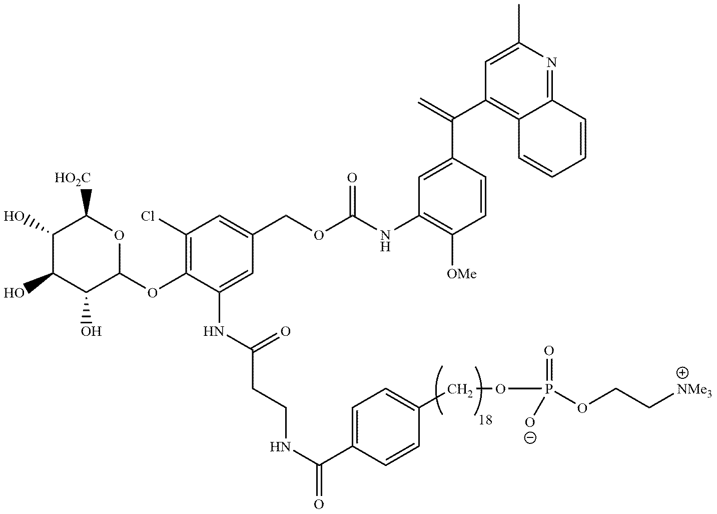 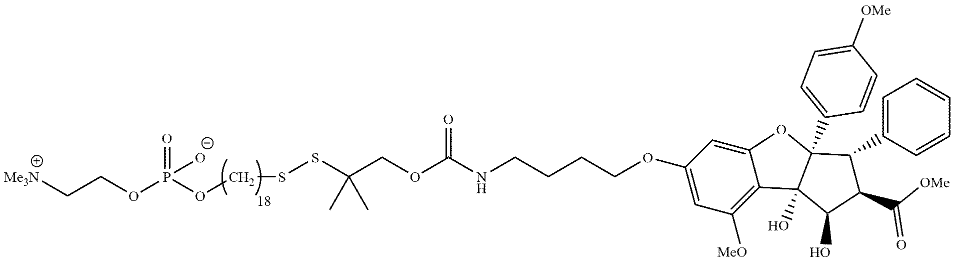 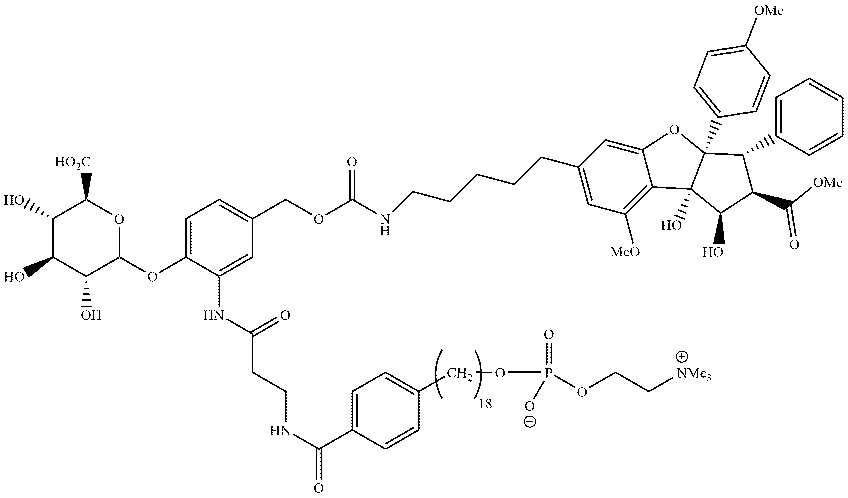
and 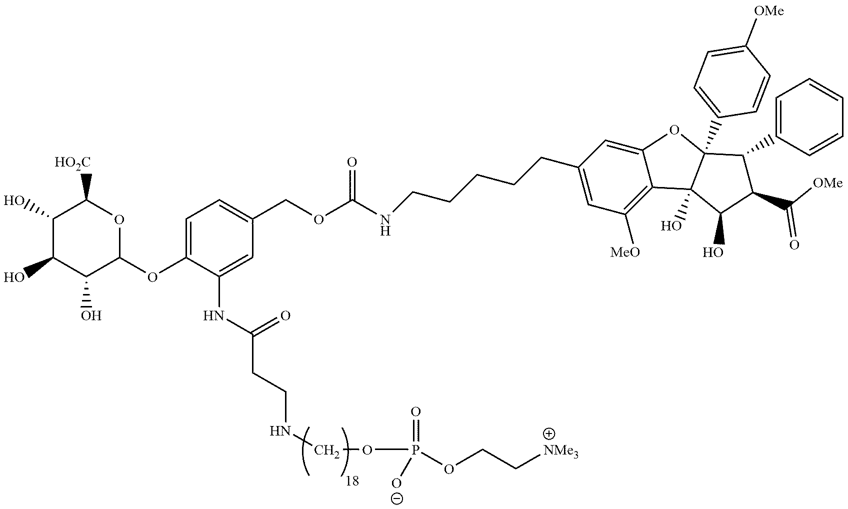
or a pharmaceutically acceptable salt thereof.

9. A pharmaceutical composition comprising a compound of claim 1, or a pharmaceutically acceptable salt thereof, and a pharmaceutically acceptable carrier.

10. A method of treating cancer in a subject in need thereof, comprising administering an effective amount of a compound of claim 1, or a pharmaceutically acceptable salt thereof.

11. The method of claim 10, wherein the cancer is melanoma, lung cancer, colorectal cancer, breast cancer, or a combination thereof.

12. The method of claim 10, wherein
the lung cancer comprises small cell lung cancer, non-small cell lung cancer, or a combination thereof;
the melanoma comprises superficial spreading melanoma, nodular melanoma, lentigo maligna melanoma, acral lentiginous melanoma, amelanotic melanoma, nevoid melanoma, spitzoid melanoma, desmoplastic melanoma, or a combination thereof;
the colorectal cancer comprises adenocarcinoma; or
the breast cancer comprises invasive breast ductal carcinoma, metastatic breast cancer, inflammatory breast cancer, triple negative breast cancer, ductal carcinoma in situ, or a combination thereof.

13. The method of claim 10, wherein the cancer comprises cancer stem cells.

14. The method of claim 10, wherein the cancer comprises metastatic cancer cells.

15. The method of claim 10, wherein the cancer comprises circulating tumor cells.

16. The method of claim 10, wherein the cancer is melanoma, lung cancer, colorectal cancer, or a combination thereof, and wherein the compound is a compound of formula (I-a), or a pharmaceutically acceptable salt thereof.

17. The method of claim 10, wherein the cancer is breast cancer, wherein the subject (1) is estrogen receptor positive, (2) is both estrogen receptor negative and progesterone receptor negative, (3) expresses HER2 (HER2+), (4) does not express HER2 (HER2-), or a combination thereof.

18. The method of claim 10, wherein the cancer is breast cancer, and wherein the compound is a compound of formula (I-b), or a pharmaceutically acceptable salt thereof.

19. The method of claim 10, wherein the cancer is cancer is melanoma, lung cancer, colorectal cancer, breast cancer, or a combination thereof, and wherein the compound is a compound of formula (I-c), or a pharmaceutically acceptable salt thereof.
